(19) United States
(12) Patent Application Publication
(10) Pub. No.: US 2012/0308608 A1
(43) Pub. Date: Dec. 6, 2012

(54) INTERFERON-INDUCING COMPOUNDS AND USES THEREOF

(76) Inventors: Megan Shaw, New York, NY (US); Mila Ortigoza, New York, NY (US); Adolfo Garcia-Sastre, New York, NY (US); Peter Palese, Leonia, NJ (US)

(21) Appl. No.: 13/576,322

(22) PCT Filed: Jan. 31, 2011
(86) PCT No.: PCT/US11/23187

§ 371 Date: Jul. 31, 2012

(51) Int. Cl. A61K 31/404; A61P 31/14; A61P 31/20; A61P 31/22; A61P 35/00; A61P 25/00; A61P 31/04; A61K 31/704; A61K 31/473; A61K 31/4745; A61K 31/428; A61K 31/4706; A61K 31/498; A61K 31/502; A61K 31/42; A61K 31/415; A61K 31/422; A61K 31/427; A61K 31/44; A61K 31/55; A61K 31/34; A61P 31/16

(52) U.S. Cl. 424/278.1; 514/418; 514/34; 514/290; 514/285; 514/367; 514/313; 514/250; 514/248; 514/378; 514/406; 514/369; 514/352; 514/217.12; 514/461

(57) ABSTRACT

Provided herein are Compounds that induce interferon production and methods for identifying such Compounds. Also provided herein are compositions comprising such Compounds and methods of using such Compounds to treat interferon-sensitive diseases such as viral infections, cancer, and multiple sclerosis.

This application claims priority benefit of U.S. provisional application No. 61/300,307, filed Feb. 1, 2010, which is incorporated herein by reference in its entirety.

This invention was made, in part, with United States Government support under award numbers R21AI083673, U54AI057158, and HHSN272200900032C awarded by the National Institutes of Health (NIH). The United States Government may have certain rights in this invention.

### 1. INTRODUCTION

Provided herein are Compounds that induce interferon production and methods for identifying such Compounds. Also provided herein are compositions comprising such Compounds and methods of using such Compounds to treat interferon-sensitive diseases such as viral infections, cancer, and multiple sclerosis.

### 2. BACKGROUND

Interferons (IFNs) are proteins made and released by the cells of most vertebrates in response to the presence of pathogens (e.g., bacteria and viruses) or tumor cells. They allow communication between cells to trigger the protective defenses of the immune system that eradicate pathogens or tumors. IFNs belong to the large class of glycoproteins known as cytokines and are typically divided among three classes: Type I IFN (IFN-I), Type II IFN (IFN-II), and Type III IFN (IFN-III).

In viral infections, the role of IFN is to render infected cells more capable of containing the infection, while warning neighboring uninfected cells to become less permissive to infection. Interferon does this by triggering an antiviral state in cells, which is characterized by the production and activity of antiviral genes known as IFN-stimulated genes (ISG). This antiviral state, if established in a timely fashion, is sufficient to contain most viral infections. For theses reasons, recombinant IFN has been effective in the treatment of several viral infections, such as vaccinia (see Rodriguez et al., Virology, 1991. 185(2):929-33), hepatitis C (see Shepherd et al., Health Technol Assess, 2004. 8(39):iii-iv, 1-125), hepatitis B (see Perrillo, Hepatology, 2009. 49(5 Suppl):S103-11), West Nile (see Anderson et al., Emerg Infect Dis, 2002. 8(1):107-8), and SARS (see Cinatl et al., Expert Opin Biol Ther, 2004. 4(6):827-36). Likewise, due to the ability of IFNs to improve the immune system response against cancer cells and act directly on cancer cells by slowing their growth and/or promoting their development into cells with more normal behavior, IFN has been administered to treat cancer patients. Interferon also has been used to treat other diseases and conditions, such as multiple sclerosis.

Direct signaling through the IFN-I pathway is important to promote the maturation of dendritic cells during viral infections (see Lopez et al., J Infect Dis, 2003. 187(7): 1126-36) and the IFN-I produced by infected or activated cells is required for the optimal priming of both CD4+ and CD8+ T cell responses (see Curtsinger et al., J Immunol, 2005. 174(8):4465-9; Havenar-Daughton et al., J Immunol, 2006. 176(6):3315-9; Kolumam et al., J Exp Med, 2005. 202(5):637-50). These studies support the idea that Compounds that induce IFN by modulating the IFN pathway can benefit both the innate and adaptive arms of the immune response to virus infection. In cancer, IFNs are believed to stimulate natural killer cells, T cells, and macrophages, thus boosting the immune system's anticancer function.

Presently, the only broad-spectrum antiviral drugs available are ribavirin and recombinant IFN. Both of these treatments are associated with toxicity which, in the case of IFN, presents a problem in cancer treatment as well. Therefore, the development of Compounds that induce endogenous IFN (and thus activate the innate immune response) would be highly beneficial in the treatment of diseases in which IFN represents a treatment option, including viral diseases cancer, and multiple sclerosis. Moreover, the increased immunogenic capacity of IFNs implies that IFN-inducting Compounds also have potential to serve as adjuvants in vaccine preparations, as well as in combination with other anti-cancer therapies.

### 3. SUMMARY

Methods for treating an interferon-sensitive disease are described involving the administration of compounds having the formulas and structures set forth herein (“Compound”) to a subject in need of such treatment. A Compound can be administered as a single-agent therapy to a subject in need of such treatment. Alternatively, a Compound can be administered in combination with one or more additional therapies to a subject in need of such treatment.

In one embodiment, provided herein is a method of treating an interferon-sensitive disease or a symptom associated therewith in a subject, comprising administering to a subject in need thereof an effective amount of a Compound. In a specific embodiment, the interferon-sensitive disease is a viral disease. In another specific embodiment, the interferon-sensitive disease is cancer. In another specific embodiment, the interferon-sensitive disease is a bacterial disease. In another specific embodiment, the interferon-sensitive disease is multiple sclerosis.

In another embodiment, provided herein is a method of preventing a symptom associated with an interferon-sensitive disease in a subject, comprising administering to a subject in need thereof an effective amount of a Compound. In a specific embodiment, the interferon-sensitive disease is a viral disease. In another specific embodiment, the interferon-sensitive disease is cancer. In another specific embodiment, the interferon-sensitive disease is a bacterial disease. In another specific embodiment, the interferon-sensitive disease is multiple sclerosis.

In a specific embodiment, provided herein is a method of inhibiting or reducing replication of a virus in a subject, comprising administering to a subject in need thereof an effective amount of a Compound.

In another specific embodiment, provided herein is a method of inhibiting or reducing replication of an influenza virus in a subject, comprising administering to a subject in need thereof an effective amount of a Compound.

In another specific embodiment, provided herein is a method of treating an influenza virus infection or a symptom associated therewith in a subject, comprising administering to a subject in need thereof an effective amount of a Compound. In another specific embodiment, provided herein is a method of preventing a symptom associated with an influenza virus infection in a subject, comprising administering to a subject in need thereof an effective amount of a Compound.

In another specific embodiment, provided herein is a method of treating an influenza virus disease in a subject, comprising administering to a subject in need thereof an effective amount of a Compound. In another specific embodiment, provided herein is a method of preventing a symptom associated with an influenza virus disease in a subject, comprising administering to a subject in need thereof an effective amount of a Compound.

In another embodiment, a Compound is used as an adjuvant. In specific embodiments, a Compound is administered as an adjuvant with a vaccine. In certain embodiments, the vaccine is a viral vaccine. In other embodiments, the vaccine is not a viral vaccine. In a specific embodiment, the vaccine is a live, attenuated viral vaccine. In another specific embodiment, the vaccine is an inactivated or killed viral vaccine.

In certain embodiments, a Compound is used to induce interferon in cultured cells. In specific embodiment, the cultured cells are cultured in a bioreactor. In a specific embodiment, a Compound is used to induce the production of recombinant interferon by cultured cells. In certain embodiments, the interferon produced by the cultured cells is isolated/purified. In certain embodiments, the interferon produced by the cultured cells is collected and used for a secondary application.

3.1 Terminology

As used herein, the term “about” or “approximately” when used in conjunction with a number refers to any number within 1, 5 or 10% of the referenced number.

As used herein, the term “Compound,” unless otherwise specified or apparent from the context, refers to a compound that induces interferon production, including compounds of formulas I, II, III, IV, and V, as well as individual compounds provided herein, (see, e.g., Section 5.1 hereof) and pharmaceutically acceptable salts, stereoisomers (including enantiomers, diastereomers, racemates and mixtures thereof), solvates, hydrates, and prodrugs thereof. In specific embodiments, a Compound induces interferon production in the presence of virus and is sometimes referred to herein as “Compound I.” In other specific embodiments, a Compound induces interferon production independent of virus infection. In other words, interferon production is induced in the presence or absence of virus infection. Such compounds are referred to herein as “Compound II.” In one embodiment, a Compound is purified/isolated.

As used herein, the term “effective amount” in the context of administering a treatment/therapy to a subject refers to the amount of a treatment which has a prophylactic and/or therapeutic effect(s). In certain embodiments, an “effective amount” in the context of administration of a treatment/therapy to a subject with an interferon-sensitive disease refers to the amount of a treatment which is sufficient to achieve one, two, three, four, or more of the following effects: (i) reduce or ameliorate the severity of an interferon-sensitive disease or a symptom or disease associated therewith; (ii) reduce the duration of an interferon-sensitive disease or a symptom or disease associated therewith; (iii) reduce or prevent the progression of an interferon-sensitive disease or a symptom or disease associated therewith; (iv) cause regression of an interferon-sensitive disease or a symptom or disease associated therewith; (v) prevent the development or onset of an interferon-sensitive disease or a symptom or disease associated therewith; (vi) reduce or prevent the recurrence of an interferon-sensitive disease or a symptom or disease associated therewith; (vii) reduce or prevent the spread of a pathogen from one cell to another cell, one tissue to another tissue, or one organ to another organ; (ix) reduce or prevent the spread of a pathogen from one subject to another subject; (x) reduce or prevent organ failure associated with an interferon-sensitive disease; (xi) reduce hospitalization of a subject; (xii) reduce hospitalization length; (xiii) increase the survival of a subject with an interferon-sensitive disease; (xiv) eliminate an interferon-sensitive disease; (xv) inhibit or reduce pathogen replication or replication of cancer cells; (xvi) inhibit or reduce the entry of a pathogen into a host cell(s); (xviii) inhibit or reduce replication of the viral genome; (xix) inhibit or reduce synthesis of viral proteins; (xx) inhibit or reduce assembly of viral particles; (xxi) inhibit or reduce release of viral particles from a host cell(s); (xxii) reduce viral titer; (xxiii) an impairment in the formation of a tumor; (xxiv) eradication, removal, or control of primary, regional and/or metastatic cancer; (xxv) the size of a tumor is maintained and does not increase or increases by less than 10%, preferably less than 5%, preferably less than 4%, preferably less than 2%; (xxvi) an increase in the number of patients in remission from cancer; (xxvii) an amelioration of interferon-sensitive disease-related symptoms and/or quality of life; (xxviii) a stabilization, reduction or elimination in the cancer cell population; (xxix) stabilization or reduction in the growth of a tumor or neoplasm; and/or (xxx) enhancement or improvement in the prophylactic or therapeutic effect(s) of another therapy.

As used herein, the term “elderly human” refers to a human 65 years or older.

As used herein, the term “human infant” refers to a newborn to 1 year old year human.

As used herein, the term “human child” refers to a human that is 1 year to 18 years old.

As used herein, the term “human adult” refers to a human that is 18 years or older.

As used herein, the term “in combination,” in the context of the administration of a Compound refers to the administration of a Compound prior to, concurrently with, or subsequent to the administration of one or more additional treatments/therapies (e.g., agents, surgery, or radiation). The use of the term “in combination” does not restrict the order in which therapies are administered to a subject. In specific embodiments, the interval of time between the administration of a Compound and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain embodiments, a Compound and one or more additional therapies are administered less than 1 day, 1 week, 2 weeks, 3 weeks, 4 weeks, one month, 2 months, 3 months, 6 months, 1 year, 2 years, or 5 years apart.

As used herein, the term “infection” means the invasion by, multiplication and/or presence of a pathogen in a cell, tissue, or subject. In one embodiment, an infection is an “active” infection, i.e., one in which the pathogen is replicating in a cell, tissue, or subject. Such an infection may be characterized by the spread of the pathogen to other cells, tissues, organs, and/or subjects from the cells, tissues, organs, and/or subjects initially infected by the pathogen. An infection may also be a latent infection, i.e., one in which the pathogen is not replicating. In one embodiment, an infection refers to the pathological state resulting from the presence of the pathogen in a cell, tissue, or subject, or by the invasion of a cell, tissue, or subject by the pathogen.

As used herein, the terms “interferon” and “IFN” refer to an interferon of any type, e.g., type I interferon, type II interferon, and type III interferon. The term interferon also refers to interferon subtypes including, but not limited to IFN-α, IFN-β and IFN-γ.

As used herein, the term “Compound that induces interferon production” describes a Compound that causes the production of interferon (e.g., production of an interferon protein).

As used herein, the term “interferon-sensitive disease” refers to a disease or condition in which the presence of and/or production of interferon has a beneficial effect. Interferon-sensitive diseases include, but are not limited to, diseases and/or infections caused by pathogens (such as viral diseases and bacterial diseases); cancers (e.g., leukemia and lymphomas including hairy cell leukemia, chronic myeloid leukemia, nodular lymphoma, cutaneous T-cell lymphoma); and autoimmune disorders (e.g., multiple sclerosis).

As used herein, the numeric term “log” refers to log₁₀.

As used herein, the phrase “multiplicity of infection” or “MOI” is the average number of pathogens per infected cell. The MOI is determined by dividing the number of pathogens added (ml added×PFU) by the number of cells added (ml added×cells/ml).

As used herein, the term “pharmaceutically acceptable salts” refer to salts prepared from pharmaceutically acceptable non-toxic acids or bases including inorganic acids and bases and organic acids and bases. Suitable pharmaceutically acceptable base addition salts for a Compound include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Other examples of salts are well known in the art, see, e.g., Remington's Pharmaceutical Sciences, 18th ed., Mack Publishing, Easton Pa. (1990).

As used herein, the term “purified,” in the context of a Compound that is chemically synthesized, refers to a Compound that is substantially free of chemical precursors or other chemicals when chemically synthesized. As used in this context, substantially means less than 40%, 35%, 30%, 25%, 20%, 15%, 10%, 5%, or 1% chemical precursors or other chemicals. In a specific embodiment, a Compound is 60%, preferably 65%, 70%, 75%, 80%, 85%, 90%, or 99% free of other, different Compounds.

As used herein, the terms “purified” and “isolated” when used in the context of a Compound that is obtained from a natural source, e.g., cells, refers to a Compound which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a Compound that has been separated from the material (e.g., cellular components of the cells) from which it is isolated. Thus, a Compound that is isolated includes preparations of a Compound having less than about 30%, 20%, 10%, 5%, 2%, or 1% (by dry weight) of cellular materials and/or contaminating materials.

As used herein, the terms “subject” or “patient” are used interchangeably. As used herein, the term “subject” refers to an animal (e.g., bird, reptile, mammal). In a specific embodiment, a subject is a mammal including a non-primate (e.g., camel, donkey, zebra, cow, pig, horse, goat, sheep, cat, dog, rat, mouse) and a primate (e.g., a monkey, chimpanzee, human). In another specific embodiment, a subject is a human. In another specific embodiment, a subject is a farm animal. In another specific embodiment, a subject is a household pet.

As used herein, unless otherwise specified, the term “substituted” means a group substituted by one to four or more substituents, such as, halo, trifluoromethyl, trifluoromethoxy, hydroxy, alkoxy, cycloalkyoxy, heterocylooxy, oxo, alkanoyl, alkylcarbonyl, cycloalkyl, aryl, aryloxy, aralkyl, alkanoyloxy, cyano, azido, amino, alkylamino, arylamino, aralkylamino, cycloalkylamino, heterocycloamino, mono and disubstituted amino in which the two substituents on the amino group are selected from alkyl, aryl, aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g., SO₂NH₂), substituted sulfonamido, nitro, carboxy, carbamyl (e.g. CONH₂), substituted carbamyl (e.g., CONH alkyl, CONH aryl, CONH aralkyl or instances where there are two substituents on the nitrogen selected from alkyl, aryl or aralkyl), alkoxycarbonyl, aryl, substituted aryl, guanidino and heterocyclo, such as, indolyl, imidazolyl, furyl, thienyl, thiazolyl, pyrrolidyl, pyridyl, pyrimidyl and the like.

As used herein, the term “premature human infant” refers to a human infant born at less than 37 weeks of gestational age.

As used herein, the terms “therapies” and “therapy” can refer to any protocol(s), method(s), composition(s), formulation(s), and/or agent(s) that can be used in the prevention, treatment, management, or amelioration of a condition or disorder or a symptom thereof (e.g., an interferon-sensitive disease or a symptom associated therewith). In certain embodiments, the terms “therapies” and “therapy” refer to a drug therapy, adjuvant therapy, radiation, surgery, biological therapy, supportive therapy, and/or other therapies useful in treatment, management, prevention, or amelioration of a condition or disorder or a symptom thereof (e.g., an interferon-sensitive disease or a symptom or disease associated therewith). In certain embodiments, the term “therapy” refers to a Compound or a pharmaceutical composition thereof. In other embodiments, the term “therapy” refers to a therapy other than a Compound or a pharmaceutical composition thereof. In specific embodiments, an “additional therapy” and “additional therapies” refer to a therapy other than a treatment using a Compound or a pharmaceutical composition thereof. In a specific embodiment, a therapy includes the use of a Compound as an adjuvant therapy. For example, using a Compound in conjunction with a drug therapy, biological therapy, surgery, and/or supportive therapy.

As used herein, unless otherwise specified, the term “C₁₋₄ alkyl” means a saturated straight chain or branched non-cyclic hydrocarbon having from 1 to 4 carbon atoms. Representative saturated straight chain C₁₋₄ alkyls include -methyl, -ethyl, -n-propyl and -n-butyl. An alkyl group can be unsubstituted or substituted.

As used herein, unless otherwise specified the term “C₂₋₄alkenyl” means a straight chain or branched non-cyclic hydrocarbon having from 2 to 4 carbon atoms and including at least one carbon-carbon double bond. Representative straight chain and branched C₂₋₄ alkenyls include -vinyl, -allyl, -1-butenyl, -2-butenyl and -isobutylenyl. The double bond of an alkenyl group can be unconjugated or conjugated to another unsaturated group. An alkenyl group can be unsubstituted or substituted.

As used herein, unless otherwise specified the term “alkoxyalkyl” means -(alkylene)-O-(alkyl), wherein each “alkyl” is independently an alkyl group as defined above, including —CH₂OCH₃, —CH₂OCH₂ CH₃, —(CH₂)₂OCH₂CH₃, —(CH₂)₂O(CH₂)₂CH₃, and the like.

As used herein, unless otherwise specified the term “aryl” means a carbocyclic aromatic ring containing from 5 to 14 ring atoms. The ring atoms of a carbocyclic aryl group are all carbon atoms. Aryl ring structures include Compounds having one or more ring structures such as mono-, bi-, or tricyclic Compounds as well as benzo-fused carbocyclic moieties such as 5,6,7,8-tetrahydronaphthyl and the like. In one embodiment, the aryl group is a monocyclic ring or bicyclic ring. Representative aryl groups include phenyl, tolyl, anthracenyl, fluorenyl, indenyl, azulenyl, phenanthrenyl and naphthyl. A carbocyclic aryl group can be unsubstituted or substituted.

As used herein, unless otherwise specified the term “heteroaryl” means a carbocyclic aromatic ring containing from 5 to 14 ring atoms and the ring atoms contain at least one heteroatom, preferably 1 to 3 heteroatoms, independently selected from nitrogen, oxygen, or sulfur. Heteroaryl ring structures include Compounds having one or more ring structures such as mono-, bi-, or tricyclic Compounds as well as fused heterocycle moities. Representative heteroaryls are triazolyl, tetrazolyl, oxadiazolyl, pyridyl, furyl, benzofuranyl, thiophenyl, benzothiophenyl, benzoisoxazolyl, benzoisothiazolyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, benzoquinazolinyl, acridinyl, pyrimidyl and oxazolyl. A group can be unsubstituted or substituted.

As used herein, unless otherwise specified the term “arylalkyl” means -(alkyl)-(aryl), wherein alkyl and aryl are defined above, including, but not limited to —(CH₂)phenyl, —(CH₂)₂phenyl, —(CH₂)₃phenyl, —CH(phenyl)₂, —CH(phenyl)₃, —(CH₂)tolyl, —(CH₂)anthracenyl, —(CH₂)fluorenyl, —(CH₂)indenyl, —(CH₂)azulenyl, —(CH₂)naphthyl, and the like.

As used herein, unless otherwise specified the term “heterocyclyl” means a monocyclic or polycyclic ring comprising carbon and hydrogen atoms, optionally having 1 to 4 multiple bonds, and the ring atoms contain at least one heteroatom, in one embodiment 1 to 3 heteroatoms, independently selected from nitrogen, oxygen, and sulfur. Heterocyclyl ring structures include Compounds having one or more ring structures such as mono-, bi-, or tricylic Compounds. In one embodiment, the heterocyclyl group is a monocyclic ring or bicyclic ring. Representative heterocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, and the like. A heterocyclyl ring can be unsubstituted or substituted.

As used herein, unless otherwise specified the term “heterocyclylalkyl” means -(alkyl)-(heterocyclyl), wherein alkyl and heterocyclyl are defined above. Substituted heterocyclylalkyl groups may be substituted at the alkyl, the heterocyclyl, or both the alkyl and the heterocyclyl portions of the group. In one embodiment, the heterocylylalkyl group is ethyl-morpholinyl.

As used herein, unless otherwise specified the term “arylcarbonyl” means —C(O)(aryl), wherein aryl is defined above, including —C(O)(phenyl), —C(O)(tolyl), —C(O)(anthracenyl), —C(O)(fluorenyl), —C(O)(indenyl), —C(O)(azulenyl), —C(O)(naphthyl), and the like.

### 4. DESCRIPTION OF THE FIGURES

FIG. 1. High throughput screen for Interferon-inducing Compounds: (A) The MDCK-IFNb-Luc reporter cell line was used to monitor Compounds that relieve the luciferase reporter from inhibition by virally encoded NS1 protein. This system is not responsive to infection with a wild type virus, e.g., A/PR/8/34 (left panel), but only to infection with a mutant NS1-containing virus (middle panel) or with a wild-type virus in the presence of an NS1 inhibitor (right panel). Positive control and positive readout cells indicate a positive induction of the reporter. (B) Response of the MDCK-IFNb-Luc reporter cell line to infection with decreasing amounts of A/PR/8/34 virus and NS1 truncated versions of this virus. Sendai virus (SeV) was used as a positive control. (C) Optimization of the screen assay in 96-well format. Results indicate four separate experiments and Z-factors are indicated. (D) Formula used to calculate Z′ factor.

FIG. 2. Screening of Compounds: (A) Pie diagram of the two classes of Compounds screened. Compounds with known biological activity made up 3.1% of the total number, while the majority of Compounds screened had unknown properties. (B) Schematic for HT Compound Screen. (C) Formula used to calculate the Z score. (D) Graph of all the average Z score of all Compound-containing wells from the primary screen. Compounds defined as hits are bracketed. Hit criteria was not based on the average Z score, but on individual Z score of each individual duplicate.

FIG. 3. Confirmation screen for hit Compounds: (A) Cytotoxicity graphs for two representative hit Compounds. (B) Dose-dependent induction of IFNb-luc reporter expression for two representative hit Compounds. The top panels depict a Compound that depends on influenza A/PR/8/34 virus infection to induce IFN, whereas the bottom panels depict a Compound that induces IFN independent of virus. ASN2 represents Compound 1 (see Table 1). ENA51 represents Compound 28 (see Table 2).

FIG. 4. Effect of IFN induced by ASN2 on VSV-GFP replication: (A) Controls for the IFN bioassay. IFN produced by Sendai virus—Cantell strain (SeV) and A/PR/8/34 NS1-113 virus, but not wild-type A/PR/8/34 virus, is able to establish an antiviral state in MDCK cells and, as a result, prevent VSV-GFP growth. IFN-treated MDCK cells and untreated cells were used as controls. (B) ASN2 is able to induce the production of IFN only if the treated cells are infected with influenza A/PR/8/34 virus. Fluorescent images of the wells are shown in the right panels for both sections, representing the growth of VSV-GFP.

FIG. 5. ASN2 attenuates influenza virus replication: Influenza A/PR/8/34 (left) and A/WSN/33 (right) virus replication is attenuated in the presence of ASN2 (Compound 1 identified in Table 1), a Compound that induces IFN only in the presence of virus, and not by itself. Viral growth is shown as HA titer at 48 (left) and 24 (right) hours post infection.

FIG. 6. ASN possesses antiviral activity: Influenza A/Vietnam/1203/2004 (H5N1) virus replication is attenuated in the presence of ASN2.

FIG. 7. Broad antiviral activity of ASN2: ASN2 is capable of inhibiting the replication of multiple strains and subtypes of influenza A virus.

FIG. 8. ASN2 affects viral replication machinery: The viral replication machinery of an influenza virus mini-genome is inhibited by ASN2, as indicated by decreasing levels of luciferase activity in the presence of increasing ASN2 concentrations.

FIG. 9. Inhibition of viral replication by ASN2 is not dependent on type I interferon: ASN2 inhibits viral replication in interferon competent cells (MDCK and A549) as well as interferon-deficient cells (Vero).

FIG. 10. Interferon induction and cytotoxicity of Compounds: 24 compounds were identified as being able to induce interferon in the absence of virus; the levels of interferon induction and cytotoxicity of the compounds is presented.

FIG. 11. Antiviral activity of Compounds in MDCK cells: The antiviral activity of compounds against vesicular stomatitis virus grown in MDCK cells is presented.

FIG. 12. Interferon Induction and antiviral activity of Compounds in human cells: Sixteen of the 24 compounds identified as being able to induce interferon in the absence of virus in MDCK cells induced interferon in human 293T and A549 cells (left panel). Four of these compounds possessed antiviral activity against vesicular stomatitis virus grown in the same human cells (right panel).

### 5. DETAILED DESCRIPTION

The present application is based, in part, on the discovery of Compounds that induce the production of interferon and that such Compounds can be used to treat interferon-sensitive diseases.

Methods for treating an interferon-sensitive disease are described involving the administration of compounds having the formulas and structures set forth herein (“Compound”) to a subject in need of such treatment. A Compound can be administered as a single-agent therapy to a subject in need of such treatment. Alternatively, a Compound can be administered in combination with one or more additional therapies to a subject in need of such treatment.

In another embodiment, a Compound is used as an adjuvant. In specific embodiments, a Compound is administered as an adjuvant with a vaccine. In certain embodiments, the vaccine is a viral vaccine. In other embodiments, the vaccine is not a viral vaccine. In a specific embodiment, the vaccine is a live, attenuated viral vaccine. In another specific embodiment, the vaccine is a non-live viral vaccine.

In certain embodiments, a Compound is used to induce interferon in cultured cells. In specific embodiment, the cultured cells are cultured in a bioreactor. In a specific embodiment, a Compound is used to induce the production of recombinant interferon by cultured cells. In certain embodiments, the interferon produced by the cultured cells is isolated/purified. In certain embodiments, the interferon produced by the cultured cells is collected and used for a secondary application.

5.1 Interferon-Inducing Compounds

5.1.1 Virus Dependent IFN-Inducing Compounds

In one embodiment, provided herein are Compounds that induce interferon in the presence of a virus (e.g., an influenza virus) (“Compound I”).

Representative examples of Compound I include the Compounds of Table 1, below.

TABLE 1

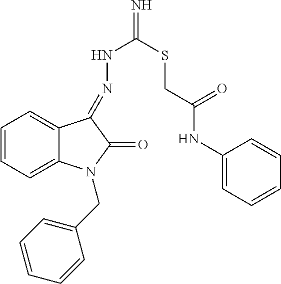

1

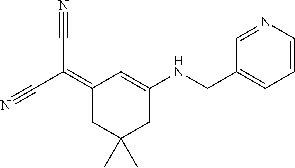

2

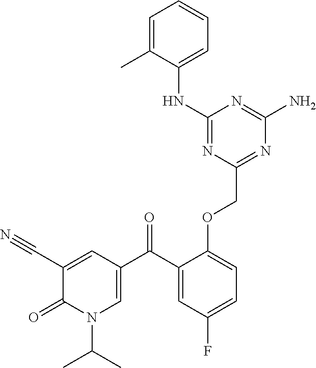

3

and pharmaceutically acceptable salts or stereoisomers, including enantiomers, diastereomers, racemates or mixtures of stereoisomers, thereof.

5.1.2 Virus Independent IFN-Inducing Compounds

In one embodiment, provided herein are Compounds that induce interferon independent of virus infection (“Compound II”).

In one embodiment, Compound II includes those of Formula I:

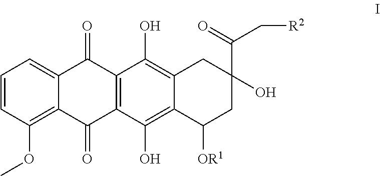

and pharmaceutically acceptable salts or stereoisomers, including enantiomers, diastereomers, racemates or mixtures of stereoisomers, thereof wherein:

R¹ is substituted or unsubstituted heterocyclyl; and

R² is H or OH;

In one embodiment, R¹ is substituted tetrahydro-2H-pyran.

In another embodiment, Compound II includes those of Formula II:

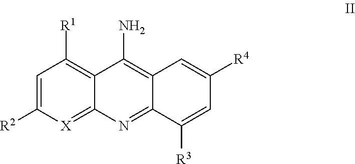

and pharmaceutically acceptable salts or stereoisomers, including enantiomers, diastereomers, racemates or mixtures of stereoisomers, thereof wherein:

X is CH or N; and

R¹, R², R³ and R⁴ are at each occurrence independently hydrogen or substituted or unsubstituted C₁₋₄ alkyl.

In one embodiment, R¹, R², R³ and R⁴ are each hydrogen.

In one embodiment, R¹, R², R³ and R⁴ are each methyl.

In another embodiment, Compound II includes those of Formula III:

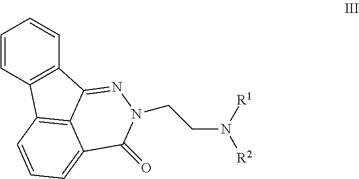

and pharmaceutically acceptable salts or stereoisomers, including enantiomers, diastereomers, racemates or mixtures of stereoisomers, thereof wherein:

R¹ and R² are at each occurrence independently substituted or unsubstituted C₁₋₄alkyl, or R¹ and R² taken together with the nitrogen atom to which they are attached form substituted or unsubstituted heterocyclyl.

In one embodiment, R¹ and R² are each ethyl.

In another embodiment, R¹ and R² taken together with the nitrogen atom to which they are attached form piperidine.

In another embodiment, Compound II includes those of Formula IV:

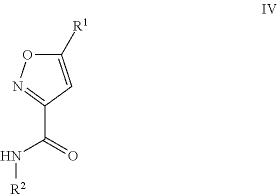

and pharmaceutically acceptable salts or stereoisomers, including enantiomers, diastereomers, racemates or mixtures of stereoisomers, thereof wherein:

R¹ is substituted or unsubstituted aryl or substituted or unsubstituted heteroaryl; and

R² is substituted or unsubstituted C₁₋₄ alkyl, substituted or unsubstituted C₂₋₄ alkenyl, substituted or unsubstituted heteroaryl or substituted or unsubstituted alkoxyalkyl.

In one embodiment, R¹ is furan.

In another embodiment, R¹ is thiophene.

In another embodiment, R¹ is phenyl.

In one embodiment, R² is arylalkyl.

In another embodiment, R² is allyl.

In another embodiment, R² is propyl.

In another embodiment, R² is alkoxyalkyl.

In another embodiment, Compound II includes those of Formula V:

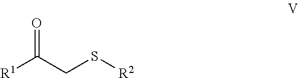

and pharmaceutically acceptable salts or stereoisomers, including enantiomers, diastereomers, racemates or mixtures of stereoisomers, thereof wherein:

R¹ is substituted or unsubstituted C₁₋₄ alkyl, substituted or unsubstituted aryl, substituted or unsubstituted heteroaryl, substituted or unsubstituted heterocyclyl, —NH(substituted or unsubstituted heteroaryl) or —NH(substituted or unsubstituted aryl); and

R² is cyano, substituted or unsubstituted heterocyclyl, substituted or unsubstituted heteroaryl, substituted or unsubstituted hydrazine or arylcarbonyl.

In one embodiment, R¹ is furan.

In another embodiment, R¹ is thiophene.

In another embodiment, R¹ is phenyl.

In another embodiment, R¹ is piperidine.

In another embodiment, R¹ is —NH(2,3-dihydrobenzo[b][1,4]dioxine).

In another embodiment, R¹ is 3,4-dihydroquinoxalin-2(1H)-one.

In another embodiment, R¹ is 1,2,3,4-tetrahydroisoquinoline.

In another embodiment, R¹ is methyl.

In one embodiment, R² is arylalkyl.

In one embodiment, R² is heterocyclylalkyl.

In another embodiment, R² is allyl.

In another embodiment, R² is propyl.

In another embodiment, R² is alkoxyalkyl.

In another embodiment, R² is 1H-pyrazolo[3,4-d]pyrimidin-4(5H)-imine.

In another embodiment, R² is 3,4-dihydroisoquinoline.

In another embodiment, R² is quinazolin-4(3H)-one.

In another embodiment, R² is 1,3,4-thiadiazole.

In another embodiment, R² is 1H-benzo[d]imidazole.

In another embodiment, R² is triazole.

In one embodiment, Compound II includes doxorubicin, doxorubicin-HCl, daunorubicin-HCl, aminacrine, acrisorcin, aklavin-HCl and ellipticine and pharmaceutically acceptable salts or stereoisomers, including enantiomers, diastereomers, racemates or mixtures of stereoisomers, thereof.

Representative examples of Compound II include the Compounds of Table 2, below.

TABLE 2

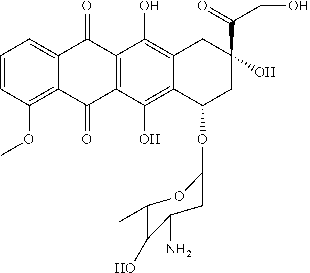

4

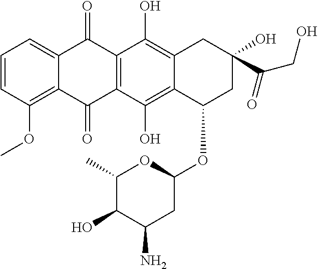

5

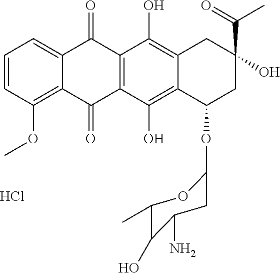

6

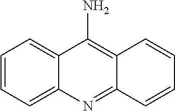

7

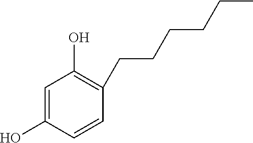

8

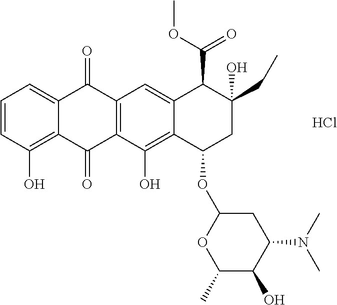

9

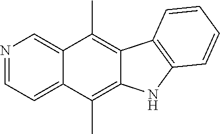

10

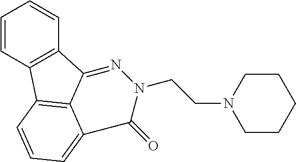

11

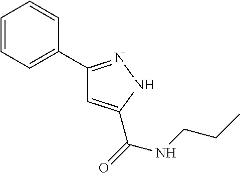

12

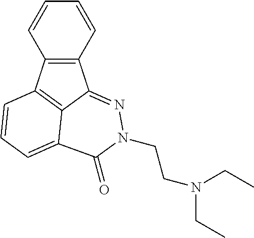

13

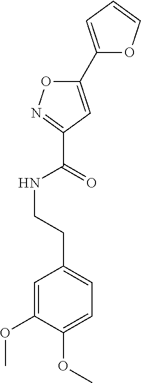

14

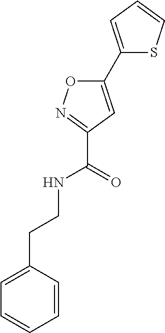

15

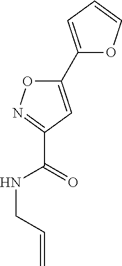

16

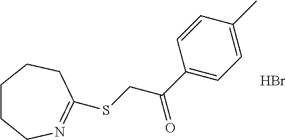

17

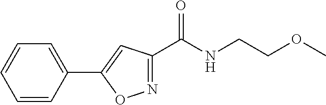

18

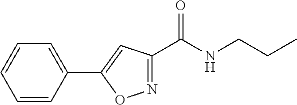

19

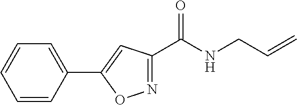

20

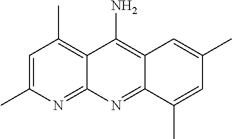

21

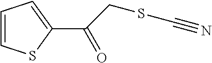

22

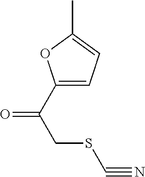

23

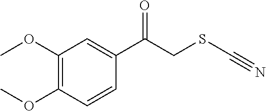

24

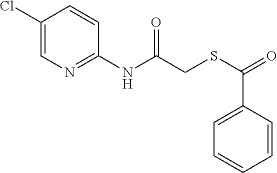

25

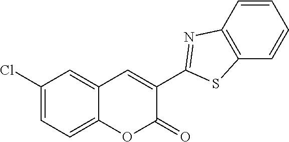

26

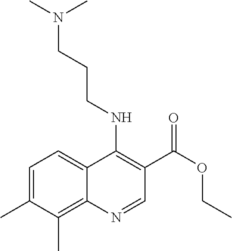

27

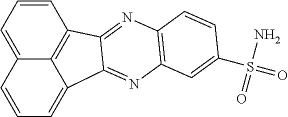

28

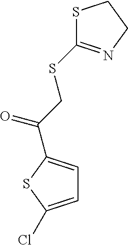

29

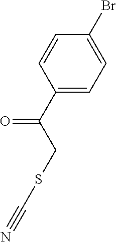

30

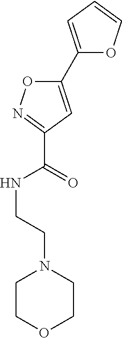

31

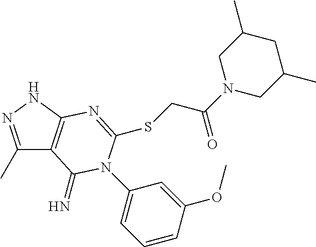

32

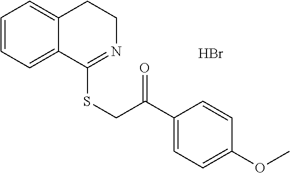

33

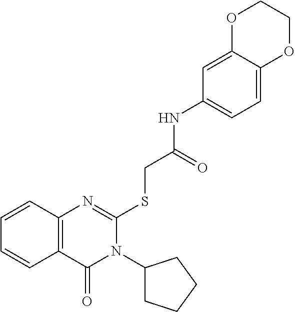

34

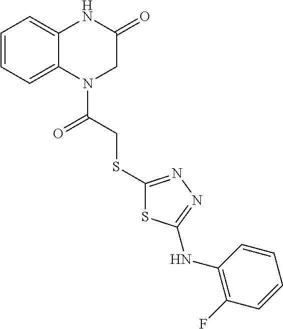

35

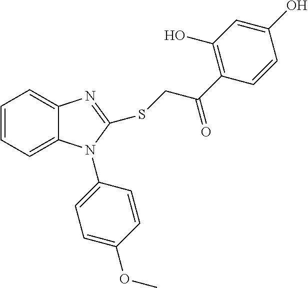

36

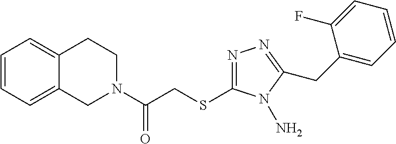

37

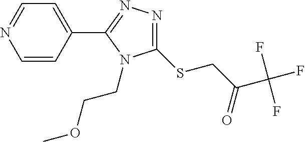

38

and pharmaceutically acceptable salts or stereoisomers, including enantiomers, diastereomers, racemates or mixtures of stereoisomers, thereof.

In certain embodiments, the Compounds do not include one or more of doxorubicin, doxorubicin-HCl, daunorubicin-HCl, aminacrine, acrisorcin, aklavin-HCl and ellipticine and pharmaceutically acceptable salts or stereoisomers, including enantiomers, diastereomers, racemates or mixtures of stereoisomers, thereof. In a particular embodiment, with respect to methods for treating an interferon-sensitive disease, the compound of formula I is not doxorubicin or daunorubicin and the compound of formula II is not aminacrine.

Compounds provided herein can be obtained via standard, well-known synthetic methodology, see e.g., March, J. Advanced Organic Chemistry; Reactions Mechanisms, and Structure, 4th ed., 1992. Starting materials useful for preparing Compounds provided herein are commercially available or can be prepared from commercially available materials using known synthetic methods and reagents.

Two or more Compounds provided herein can be administered in combination with each other, either concomitantly or sequentially. In a particular embodiment, aminacrine and acriorcine are administered in combination.

5.2 Biological Assays

5.2.1 Screening Assays For Assessing Interferon Production

The ability of a compound to induce interferon production can be assessed by any method known in the art for measuring protein levels, such as immunoprecipitation, Western blot analysis (immunoblotting), enzyme-linked immunosorbent assay (ELISA), quantitative protein assays, protein activity assays (for example, histone deacetylase activity), immunohistochemistry, immunocytochemistry or fluorescence-activated cell sorting (FACS). Antibodies directed to a target, e.g., interferon, can be identified and obtained from a variety of sources, such as the MSRS catalog of antibodies (Aerie Corporation, Birmingham, Mich.), or can be prepared via conventional monoclonal or polyclonal antibody generation methods well known in the art.

In other embodiments, levels of interferon production are assessed by measuring levels of interferon at the RNA level using methods known to those of skill in the art. For example, RNA levels can be quantified by routine methods such as of Northern blot analysis, competitive polymerase chain reaction (PCR), or quantitative (e.g., real-time) PCR. RNA analysis can be performed on a selected nucleic acid population, for example, total cellular and/or viral RNA, RNAs with a certain size cut-off, etc.

In a specific embodiment, the ability of a compound to induce interferon production is assessed using a modified functional interferon bioassay (see, e.g., Park et al., J Virol, 2003. 77(2):1501-11; and Iwata et al., J Vet Med Sci, 1996. 58(1)23-7). In accordance with such an assay, wells of plates are seeded with cells, e.g., MDCK cells, and the cells are allowed to incubate for ˜20-24 hours at 37° C., 5% CO₂. The cells then are treated with compounds of interest. Two hours after treatment, the cells are either mock infected or infected with virus added directly into the media and incubated for ˜18-20 hours at 37-C, 5% CO₂. Supernatant from the treated/infected cells (containing interferon) is transferred into a new plate and UV inactivated. Dilutions are made with the UV-inactivated supernatants. These dilutions are overlaid onto fresh cells that are plated the previous day, and left to incubate for ˜20-24 hours at 37° C., 5% CO₂. These cells are then infected with a reporter protein (e.g., GFP)-expressing virus that is sensitive to the effects of IFN and will only replicate if the supernatant from the cells previously infected with virus do not contain IFN protein. Replication of the reporter protein-expressing viruses, such as GFP-expressing viruses, can be is monitored by measuring the relative fluorescence of the reporter protein in a plate reader, thus allowing for determination of whether IFN is present in the supernatant of the virus-infected cells and thus whether the compound induced IFN production.

In another embodiment, the ability of a compound to induce interferon production is assessed using an assay comprising cells engineered to express a reporter gene (e.g., a luciferase reporter gene) operably linked to an interferon promoter. In accordance with such assays, a cell expressing a reporter gene (e.g., a luciferase reporter gene) may be contacted with a compound. The ability of the compound to induce interferon production then can be assessed by measuring the level of reporter gene activity in the presence of the compound and comparing it to the level of reporter gene activity in the absence of a compound or in the presence of a negative control. Methods for measuring reporter gene activity are well-known in the art.

The assays and parameters presented hereinabove for selecting a compound can be used to identify compounds that induce interferon production.

5.2.2 Cytotoxicity Assays

In specific embodiments, Compounds preferentially affect the viability of cells affected by an interferon-sensitive disease. In specific embodiments, Compounds are not so cytotoxic that they are unsafe for administration to an animal or human subject with an interferon-sensitive disease.

Many assays well-known in the art can be used to assess viability of cells or cell lines following exposure to a Compound and, thus, determine the cytotoxicity of the Compound. For example, cell proliferation can be assayed by measuring Bromodeoxyuridine (BrdU) incorporation (See, e.g., Hoshino et al., 1986, Int. J. Cancer 38, 369; Campana et al., 1988, J. Immunol. Meth. 107:79), (3H) thymidine incorporation (See, e.g., Chen, J., 1996, Oncogene 13:1395-403; Jeoung, J., 1995, J. Biol. Chem. 270:18367 73), by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability.

In specific embodiments, cell viability is measured in three-day and seven-day periods using an assay standard in the art, such as the CellTiter-Glo Luminescent Cell Viability Assay Kit (Promega, Madison, Wis.) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes. These changes are given a designation of T (100% toxic), PVH (partially toxic-very heavy—80%), PH (partially toxic-heavy—60%), P (partially toxic-40%), Ps (partially toxic-slight—20%), or 0 (no toxicity—0%), conforming to the degree of cytotoxicity seen. A 50% cell inhibitory (cytotoxic) concentration (CC₅₀) is determined by regression analysis of these data. In certain embodiments, compounds have an CC₅₀ of at least 150 μM, 175 μM, 200 μM, 225 μM, 250 μM, 300 μM, 325 μM, or 350 μM. In certain embodiments, compounds have an CC₅₀ in the range of 150 μM to 200 μM, 150 μM to 300 μM, 200 μM to 300 μM, 250 μM to 300 μM, 300 μM to 400 μM, 350 μM to 450 μM, or 375 μM to 500 μM.

In a specific embodiment, the cells used in the cytotoxicity assay are animal cells, including primary cells and cell lines. In some embodiments, the cells are human cells. In certain embodiments, cytotoxicity is assessed in one or more of the following cell lines: U937, a human monocyte cell line; primary peripheral blood mononuclear cells (PBMC); Huh7, a human hepatoblastoma cell line; 293T, a human embryonic kidney cell line; or THP-1, monocytic cells. In certain embodiments, cytotoxicity is assessed in one or more of the following cell lines: MDCK, MEF, Huh 7.5, Detroit, or human tracheobronchial epithelial (HTBE) cells.

Compounds can be tested for in vivo toxicity in animal models. For example, animal models, described herein and/or known in the art, used to test the activities of Compounds can also be used to determine the in vivo toxicity of these Compounds. For example, animals are administered a range of concentrations of Compounds. Subsequently, the animals are monitored over time for lethality, weight loss or failure to gain weight, and/or levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and/or regimen in addition to dosages.

The toxicity and/or efficacy of a Compound can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A Compound that exhibits large therapeutic indices is preferred. While a Compound that exhibits toxic side effects may be used, care should be taken to design a delivery system that targets such agents to the site of affected tissue in order to minimize potential damage to uninfected cells and, thereby, reduce side effects. The selective index of Compounds can also be assessed by determining the cytotoxic index (CC₅₀)/IC₅₀ ratio. In certain embodiments, the selective index (CC₅₀/IC₅₀) of Compounds is at least 25, 50, 75, 100, 125, or 150. In certain embodiments, the selective index (CC₅₀/IC₅₀) of Compounds is in the range of 50 to 75, 50 to 100, or 75 to 125.

The data obtained from the cell culture assays and animal studies can be used in formulating a range of dosage of a Compound identified in accordance with the embodiments described herein for use in humans. The dosage of such agents lies preferably within a range of circulating concentrations that include the ED₅₀ with little or no toxicity. The dosage may vary within this range depending upon the dosage form employed and the route of administration utilized. For any Compound used in the methods and compositions described herein, the therapeutically effective dose can be estimated initially from cell culture assays. A dose may be formulated in animal models to achieve a circulating plasma concentration range that includes the IC₅₀ (i.e., the concentration of the test Compound that achieves a half-maximal inhibition of symptoms) as determined in cell culture. Such information can be used to more accurately determine useful doses in humans. Levels in plasma may be measured, for example, by high-performance liquid chromatography.

5.2.3 Antiviral Assays

5.2.3.1 Cellular Assays for Assessing the Effect of a Compound on Viral Replication

The effect of a Compound on virus replication can be assessed by any assay known in the art. The cells can be infected at different MOIs and the effect of a Compound on virus replication can be assessed. For example, the MOIs may be 0.001, 0.005, 0.01, 0.05, 0.1, 0.5, 1, 2.5, or 5 or greater. The effect of different concentrations of a Compound on virus replication can also be assessed. The cells or other substrate that contains cells (e.g., embryonated eggs) used in the assay should be susceptible to infection by the virus (e.g., influenza virus). The cells may be primary cells or established cell lines. In one embodiment, the cell or cell line is biologically relevant to virus infection.

Virus replication can be measured at different times post-infection. For example, virus replication may be measured 6 hours, 12 hours, 16 hours, 24 hours, 48 hours or 72 hours post-infection. Any method known to one of skill in the art can be used measure virus replication.

In certain embodiments, a Compound is considered to reduce or inhibit viral replication if it inhibits or reduces viral replication (i.e., the production of viral particles) by at least 1.5 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 100 fold, 500 fold, or 1000 fold relative to virus replication in the absence of Compound or the presence of a negative control. In certain embodiments, a Compound is considered to reduce or inhibit viral replication if it reduces the virus replication by 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to virus replication in the absence of Compound or the presence of a negative control. In some embodiments, a Compound is considered to reduce or inhibit viral replication if it reduces the virus replication by approximately 2 logs or more, approximately 3 logs or more, approximately 4 logs or more, approximately 5 logs or more, or 2 to 10 logs or 2 to 5 logs relative to virus replication in the absence of Compound or the presence of a negative control.

In certain embodiments, a Compound is considered to reduce or inhibit viral replication if it reduces the replication of a viral genome by about at least 1.5 fold, 2, fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 500 fold, or 1000 fold relative to replication of the viral genome in the absence of a Compound or relative to a negative control in an assay described herein or others known to one of skill in the art. In certain embodiments, a Compound is considered to reduce or inhibit viral replication if it reduces the replication of a viral genome by about 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to replication of the viral genome in the absence of a Compound or relative to a negative control in an assay described herein or others known to one of skill in the art. In certain embodiments, a Compound is considered to reduce or inhibit viral replication if it reduces the replication of a viral genome by at least 1 log, 1.5 logs, 2 logs, 2.5 logs, 3 logs, 3.5 logs, 4 logs, 4.5 logs, 5 logs or more relative to replication of the viral genome in the absence of a Compound or relative to a negative control in an assay described herein or others known to one of skill in the art.

In certain embodiments, a Compound is considered to reduce or inhibit viral replication if it reduces the synthesis of viral proteins by at least 1.5 fold, 2, fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 500 fold, or 1000 fold relative to the synthesis of viral proteins in the absence of a Compound or relative to a negative control in an assay described herein or others known to one of skill in the art in an assay described herein or others known to one of skill in the art. In certain embodiments, a Compound is considered to reduce or inhibit viral replication if it reduces the synthesis of viral proteins at least 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to the synthesis of viral proteins in the absence of a Compound or relative to a negative control in an assay described herein or others known to one of skill in the art. In certain embodiments, a Compound is considered to reduce or inhibit viral replication if it reduces the synthesis of viral proteins approximately 1 log, 1.5 logs, 2 logs, 2.5 logs, 3 logs, 3.5 logs, 4 logs, 4.5 logs, 5 logs relative to the synthesis of viral proteins in the absence of a Compound or relative to a negative control in an assay described herein or others known to one of skill in the art.

In certain embodiments, a Compound is considered to reduce or inhibit viral replication if it results in 1.5 fold or more, 2 fold or more, 3 fold or more, 4 fold or more, 5 fold or more, 6 fold or more, 7 fold or more, 8 fold or more, 9 fold or more, 10 fold or more, fold or more, 20 fold or more, 25 fold or more, 30 fold or more, 35 fold or more, 40 fold or more, 45 fold or more, 50 fold or more, 60 fold or more, 70 fold or more, 80 fold or more, 90 fold or more, or 100 fold or more reduction of viral yield per round of viral replication. In certain embodiments, a Compound results in about a 2 fold or more reduction of viral yield per round of viral replication. In certain embodiments, a Compound is considered to reduce or inhibit viral replication if it results in a 2 to 4, 2 to 5, 3 to 5, or 5 to 10 fold reduction of viral yield per round of viral replication. In a specific embodiment, a Compound results in about a 10 fold or more reduction of viral yield per round of viral replication.

In certain embodiments, a Compound is considered to reduce or inhibit viral replication if it reduces viral replication by at least 2 wells of hemagglutinin (HA) in a hemagglutination assay, which equals approximately a 75% reduction in viral titer.

In certain embodiments, a Compound is considered to reduce or inhibit viral replication if it reduces viral titer by 50% or more, by 55% or more, by 60% or more, by 65% or more, by 70% or more, by 75% or more, by 80% or more, by 85% or more, by 90% or more, or by 95% or more. In certain embodiments, a Compound is considered to reduce or inhibit viral replication if it reduces viral titer approximately 1 log, 1.5 logs, 2 logs, 2.5 logs, 3 logs, 3.5 logs, 4 logs, 4.5 logs, 5 logs relative to the viral titer in the absence of a Compound or relative to a negative control in an assay described herein or others known to one of skill in the art. In certain embodiments, a Compound is considered to reduce or inhibit viral replication if it reduces viral titer approximately 2 to 5, 3 to 5, 4 to 5, or 5 to 10 logs relative to the viral titer in the absence of a Compound or relative to a negative control in an assay described herein or others known to one of skill in the art.

In some embodiments, the effect of a Compound on the replication of an influenza virus is determined. Standard assays for influenza virus replication have been described (see, e.g., Sidwell et al., Antiviral Research, 2000, 48:1-16). In some embodiments, the effect of a Compound on the replication of an influenza A virus is determined. In some embodiments, the effect of a Compound on the replication of an influenza B virus is determined. In some embodiments, the effect of a Compound on the replication of an influenza C virus is determined. In some embodiments, the effect of a Compound on the replication of a currently circulating influenza virus is determined. In some embodiments, the effect of a Compound on replication of H1N1 influenza virus is determined. In some embodiments, the effect of a Compound on replication of H5N1 influenza virus is determined. In some embodiments, the effect of a Compound on replication of an attenuated influenza virus is determined. In some embodiments, the effect of a Compound on the replication of a naturally occurring strain, variant or mutant of an influenza virus, a mutagenized influenza virus, a reassortant influenza virus and/or a genetically engineered influenza virus can be assessed. In a specific embodiment, the effect of a Compound on the replication of a vaccine strain of an influenza virus is determined.

5.2.3.1.1 Viral Titer Assays

The effect of a Compound on virus can be assessed using viral titer assays. In an exemplary viral titer assay, a monolayer of a target mammalian cell line is infected with different amounts (e.g., multiplicity of 3 plaque forming units (pfu) or 5 pfu) of virus and subsequently cultured in the presence or absence of various dilutions of a Compound (e.g., 0.1 μg/mL, 1 μg/mL, 5 μg/mL, or 10 μg/mL) to be tested. Infected cultures are harvested after a certain period of time (e.g., 48 hours or 72 hours) post infection and titered by standard plaque assays known in the art on the appropriate target cell line (e.g., Vero cell, MDCK cell, MBCK cell, human respiratory epithelial cell (e.g., A549 cells), HEK 293 cell, 293T cells, calf kidney cell or mink lung cell). In certain embodiments, culturing the infected cells in the presence of a Compound reduces the yield of infectious virus by at least 1.5 fold, 2, fold, 3, fold, 4 fold, 5 fold, 6 fold, 7 fold, 8 fold, 9 fold, 10 fold, 15 fold, 20 fold, 25 fold, fold, 35 fold, 40 fold, 45 fold, 50 fold, 100 fold, 500 fold, or 1000 fold relative to culturing the infected cells in the absence of Compounds.

In certain embodiments, culturing the infected cells in the presence of a Compound reduces the yield of infectious virus by at least 1 log, 1.5 logs, 2 logs, 2.5 logs, 3 logs, 3.5 logs, 4 logs, 4.5 logs, 5 logs, 6 logs, 7, logs, 8 logs, or 9 logs relative to culturing the infected cells in the absence of a Compound. In a specific embodiment, culturing the infected cells in the presence of a Compound reduces the yield of infectious virus by at least 1 log or 2 log relative to culturing the infected cells in the absence of the Compound. In a specific embodiment, culturing the infected cells in the presence of a Compound reduces the yield of infectious virus by 2 to 3, 2 to 4, 2 to 5, or 5 to 10 log relative to culturing the infected cells in the absence of the Compound. In another specific embodiment, culturing the infected cells in the presence of a Compound reduces the yield of infectious virus by at least 2 log relative to culturing the infected cells in the absence of the Compound.

5.2.3.1.2 Plaque Reduction Assay

In a non-limiting example of a plaque reduction assay, a virus is diluted into various concentrations and added to each well containing a monolayer of the target mammalian cells in triplicate. The plates are then incubated for a period of time to achieve effective infection of the control sample (e.g., 1 hour with shaking every fifteen minutes). After the incubation period, an equal amount of 1% agarose is added to an equal volume of each Compound dilution prepared in 2× concentration. In certain embodiments, Compounds at test concentrations between about 0.03 μg/ml to about 100 μg/ml can be tested with a final agarose overlay concentration of 0.5%. The Compound-agarose mixture is applied to each well in 2 ml volume and the plates are incubated for three days, after which the cells are stained with a 1.5% solution of neutral red. At the end of the 4-6 hour incubation period, the neutral red solution is aspirated, and plaques counted using a stereomicroscope. Alternatively, a final agarose concentration of 0.4% can be used. In other embodiments, the plates are incubated for more than three days with additional overlays being applied on day four and on day 8 when appropriate. In another embodiment, the overlay medium is liquid rather than semi-solid.

5.2.3.1.3 Flow Cytometry

Flow cytometry can be utilized to detect expression of virus antigens in infected target cells cultured in the presence or absence of Compounds (See, e.g., McSharry et al., Clinical Microbiology Rev., 1994, 7:576-604). Non-limiting examples of viral antigens that can be detected on cell surfaces by flow cytometry include, but are not limited to HA of influenza. In other embodiments, intracellular viral antigens or viral nucleic acid can be detected by flow cytometry with techniques known in the art.

5.2.3.1.4 Viral Cytopathic Effect (CPE)

CPE is the morphological changes that cultured cells undergo upon being infected by most viruses. These morphological changes can be observed easily in unfixed, unstained cells by microscopy. Forms of CPE, which can vary depending on the virus, include, but are not limited to, rounding of the cells, appearance of inclusion bodies in the nucleus and/or cytoplasm of infected cells, and formation of syncytia, or polykaryocytes (large cytoplasmic masses that contain many nuclei).

The CPE assay can provide a measure of the effect of a Compound on virus replication. In a non-limiting example of such an assay, Compounds are serially diluted (e.g. 1000, 500, 100, 50, 10, 1 μg/ml) and added to 3 wells containing a cell monolayer (preferably mammalian cells at 80-100% confluent) of a 96-well plate. Within 5 minutes, viruses are added and the plate sealed, incubated at 37° C. for the standard time period required to induce near-maximal viral CPE (e.g., approximately 48 to 120 hours, depending on the virus and multiplicity of infection). When assaying a Compound for its potential activity, CPE is read microscopically after a known positive control drug (an antiviral) is evaluated in parallel with Compounds in each test. A non-limiting example of a positive control is ribavirin for influenza. The data is expressed as 50% effective concentrations or approximated virus-inhibitory concentration, 50% endpoint (IC₅₀) and cell-inhibitory concentration, 50% endpoint (CC₅₀). General selectivity index (“SI”) is calculated as the CC₅₀ divided by the IC₅₀. These values can be calculated using any method known in the art, e.g., the computer software program MacSynergy II by M. N. Prichard, K. R. Asaltine, and C. Shipman, Jr., University of Michigan, Ann Arbor, Mich.

In one embodiment, a Compound has an SI of greater than 3, 4, 5, 6, 7, 8, 9, 10, 11, 12, 13, 14, 15, 20, 21, 22, 23, 24, 25, 30, 35, 39, 40, 45, 50, 60, 70, 80, 90, 100, 200, 300, 400, 500, 1,000, or 10,000. In some embodiments, a Compound has an SI of greater than 10. In a specific embodiment, Compounds with an SI of greater than 10 are further assessed in other in vitro and in vivo assays described herein or others known in the art to characterize safety and efficacy.

5.2.3.1.5 Neutral Red NR Dye Uptake assay

The Neutral Red NR Dye Uptake assay can be used to validate the CPE inhibition assay. In a non-limiting example of such an assay, the same 96-well microplates used for the CPE inhibition assay can be used. Neutral red is added to the medium, and cells not damaged by virus take up a greater amount of dye. The percentage of uptake indicating viable cells is read on a microplate autoreader at dual wavelengths of 405 and 540 nm, with the difference taken to eliminate background. (See McManus et al., Appl. Environment. Microbiol. 31:35-38, 1976). An EC₅₀ is determined for samples with infected cells and contacted with Compounds, and an IC₅₀ is determined for samples with uninfected cells contacted with Compounds.

5.2.3.1.6 Virus Yield Assay

Lysed cells and supernatants from infected cultures such as those in the CPE assay can be used to assay for virus yield (production of viral particles after the primary infection). In a non-limiting example, these supernatants are serially diluted and added onto monolayers of susceptible cells (e.g., Vero cells). Development of CPE in these cells is an indication of the presence of infectious viruses in the supernatant. The 90% effective concentration (EC₉₀), the Compound concentration that inhibits virus yield by 1 log, is determined from these data using known calculation methods in the art. In one embodiment, the EC₉₀ of a Compound is at least 1.5 fold, 2 fold, 3 fold, 4 fold, 5 fold, 6 fold, 7 fold, 8 fold, 9 fold, 10 fold, 20 fold, 30 fold, 40 fold, or 50 fold less than the EC₉₀ of the negative control sample.

5.2.3.1.7 Cell Lines for Assays

In a certain embodiments, cells used in the antiviral assays described herein are susceptible to infection with a virus. In some embodiments, cell lines for use in antiviral assays are genetically engineered to render them more suitable hosts for viral infection or viral replication and more convenient substrates for rapidly detecting virus-infected cells (See, e.g., Olivo, P. D., Clin. Microbiol. Rev., 1996, 9:321-334). Exemplary cell lines for use in assessing the ability of Compounds to inhibit influenza replication include, without limitation, Vero cells, 293T cells, and A549 cells.

5.2.3.2 Animal Assays

Compounds and compositions are preferably assayed in vivo for the desired therapeutic or prophylactic activity prior to use in humans. For example, in vivo assays can be used to determine whether it is preferable to administer a Compound and/or another therapy. For example, to assess the use of a Compound to prevent a viral infection, the Compound can be administered before the animal is infected with the virus. Alternatively, or in addition, a Compound can be administered to the animal at the same time that the animal is infected with the virus. To assess the use of a Compound to treat or manage a viral infection, in one embodiment, the Compound is administered to animal after a viral infection. In another embodiment, a Compound is administered to the animal at the same time that the animal is infected with the virus to treat and/or manage the viral infection. In a specific embodiment, the Compound is administered to the animal more than one time.

Compounds can be tested for antiviral activity against virus in animal models systems including, but are not limited to, rats, mice, chicken, cows, monkeys, pigs, goats, sheep, dogs, rabbits, guinea pigs, etc. In a specific embodiment, Compounds are tested in a mouse model system. Such model systems are widely used and well-known to the skilled artisan. Compounds can also be tested for replication enhancing activity toward virus replication in animal models systems including, but are not limited to, rats, mice, chicken, cows, monkeys, pigs, goats, sheep, dogs, rabbits, guinea pigs, etc.

Animals are infected with virus and concurrently or subsequently treated with a Compound or placebo. Alternatively, animals are treated with a Compound or placebo and subsequently infected with virus. Samples obtained from these animals (e.g., serum, urine, sputum, semen, saliva, plasma, or tissue sample) can be tested for viral replication via well known methods in the art, e.g., those that measure altered viral titers (as determined, e.g., by plaque formation), the production of viral proteins (as determined, e.g., by Western blot, ELISA, or flow cytometry analysis) or the production of viral nucleic acids (as determined, e.g., by RT-PCR or northern blot analysis). For quantitation of virus in tissue samples, tissue samples are homogenized in phosphate-buffered saline (PBS), and dilutions of clarified homogenates are adsorbed for 1 hour at 37° C. onto monolayers of cells (e.g., Vero, CEF or MDCK cells). In other assays, histopathologic evaluations are performed after infection, preferably evaluations of the organ(s) the virus is known to target for infection. Virus immunohistochemistry can be performed using a viral-specific monoclonal antibody.

The effect of a Compound on a virus can also be determined using in vivo assays in which the titer of the virus in an infected subject administered a Compound, the length of survival of an infected subject administered a Compound, the immune response in an infected subject administered a Compound, the number, duration and/or severity of the symptoms in an infected subject administered a Compound, and/or the time period before onset of one or more symptoms in an infected subject administered a Compound is assessed. Techniques known to one of skill in the art can be used to measure such effects.

Testing may be performed in normal animals, or in experimental virus disease models known in the art. For administration to animals, Compounds are formulated in a pharmaceutically acceptable carrier. Administration includes any suitable route of administration, such as parenteral, intraperitoneal, intravenous, pulmonary, intranasally, topically, and subcutaneous.

5.2.3.2.1 Influenza Virus Animal Models

Animal models, such as ferret, mouse, guinea pig, and chicken, developed for use to test antiviral agents against influenza virus have been described (see, e.g., Sidwell et al., Antiviral Res., 2000, 48:1-16; Lowen A. C. et al. PNAS, 2006, 103: 9988-92; and McCauley et al., Antiviral Res., 1995, 27:179-186). For mouse models of influenza, non-limiting examples of parameters that can be used to assay antiviral activity of a Compound administered to the influenza-infected mice include pneumonia-associated death, serum α1-acid glycoprotein increase, animal weight, lung virus assayed by hemagglutinin, lung virus assayed by plaque assays, and histopathological change in the lung. Statistical analysis is carried out to calculate significance (e.g., a P value of 0.05 or less).

Nasal turbinates and trachea may be examined for epithelial changes and subepithelial inflammation. The lungs may be examined for bronchiolar epithelial changes and peribronchiolar inflammation in large, medium, and small or terminal bronchioles. The alveoli are also evaluated for inflammatory changes. The medium bronchioles are graded on a scale of 0 to 3+ as follows: 0 (normal: lined by medium to tall columnar epithelial cells with ciliated apical borders and basal pseudostratified nuclei; minimal inflammation); 1+(epithelial layer columnar and even in outline with only slightly increased proliferation; cilia still visible on many cells); 2+ (prominent changes in the epithelial layer ranging from attenuation to marked proliferation; cells disorganized and layer outline irregular at the luminal border); 3+ (epithelial layer markedly disrupted and disorganized with necrotic cells visible in the lumen; some bronchioles attenuated and others in marked reactive proliferation).

The trachea is graded on a scale of 0 to 2.5+ as follows: 0 (normal: Lined by medium to tall columnar epithelial cells with ciliated apical border, nuclei basal and pseudostratified. Cytoplasm evident between apical border and nucleus. Occasional small focus with squamous cells); 1+ (focal squamous metaplasia of the epithelial layer); 2+(diffuse squamous metaplasia of much of the epithelial layer, cilia may be evident focally); 2.5+ (diffuse squamous metaplasia with very few cilia evident).

Virus immunohistochemistry is performed using a viral-specific monoclonal antibody (e.g. NP-, N- or HN-specific monoclonal antibodies). Staining is graded 0 to 3+ as follows: 0 (no infected cells); 0.5+ (few infected cells); 1+ (few infected cells, as widely separated individual cells); 1.5+ (few infected cells, as widely separated singles and in small clusters); 2+ (moderate numbers of infected cells, usually affecting clusters of adjacent cells in portions of the epithelial layer lining bronchioles, or in small sublobular foci in alveoli); 3+ (numerous infected cells, affecting most of the epithelial layer in bronchioles, or widespread in large sublobular foci in alveoli).

5.2.3.3 Assays in Humans

In one embodiment, a Compound is assessed in human subjects infected with virus. In accordance with this embodiment, a Compound is administered to the human subject, and the effect of the Compound on viral replication can be measured by, e.g., analyzing the level of the virus or viral nucleic acids in a biological sample from the human subject (e.g., serum or plasma). The effect of a Compound on viral replication can be assessed by comparing the level of virus replication in a subject or group of subjects treated with a control Compound to that in a subject or group of subjects treated with a Compound. Alternatively, alterations in viral replication can be identified by comparing the level of the virus replication in a subject or group of subjects before and after the administration of a Compound. Techniques known to those of skill in the art can be used to obtain the biological sample and analyze the mRNA or protein expression.

In another embodiment, the effect of a Compound on the severity of one or more symptoms associated with a virus infection/disease are assessed in an infected subject. In accordance with this embodiment, a Compound or a control Compound is administered to a human subject suffering from a virus infection and the effect of the Compound on one or more symptoms of the virus infection is determined. The effect of a compound on the severity of one or more symptoms of a virus infection/disease can be determined by comparing the subjects treated with a control compound to the subjects treated with the Compound. Techniques known to physicians familiar with infectious diseases can be used to determine whether a Compound reduces one or more symptoms associated with the a viral disease. In other embodiments, the length of survival, frequency of hospitalization, and/or length of hospitalization of subjects with a virus infection/disease can be assessed.

5.2.4 Anticancer Assays

5.2.4.1 In Vitro Assays

A Compound can be tested in vitro and/or in vivo for their ability to reduce the amount of cancer cells, or inhibit their proliferation. The ability of a Compound to reduce the amount of cancer cells or inhibit their proliferation can be assessed by various approaches including, but not limited to: detecting the expression of antigens on cancer cells; detecting the proliferation or viability of cancer cells; and detecting the effector function of cancer cells. Techniques known to those skilled in the art can be used for measuring these activities. For example, cellular proliferation can be assayed by ³H-thymidine incorporation assays and trypan blue cell counts. Antigen expression can be assayed, for example, by immunoassays including, but not limited to, competitive and non-competitive assay systems using techniques such as Western blots, immunohistochemistry, radioimmunoassays, ELISA, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and FACS analysis.

A Compound is preferably tested in vitro and then in vivo for the desired therapeutic or prophylactic activity prior to use in humans. For example, assays which can be used to determine whether administration of a specific Compound is indicated include cell culture assays in which a patient tissue sample (e.g., cancer cells) is grown in culture and exposed to, or otherwise contacted with, a Compound, and the effect of such Compound upon the tissue sample is observed. The tissue sample can be obtained by, e.g., biopsy from the patient.

5.2.4.2 In Vivo Assays

A Compound can be tested in suitable animal model systems prior to use in humans for the treatment of cancer. Such animal model systems include, but are not limited to, rats, mice, chicken, cows, monkeys, pigs, dogs, rabbits, etc. Any animal system well-known in the art may be used. Several aspects of the procedure may vary; said aspects include, but are not limited to, the temporal regime of administering a Compound, whether such a Compound is administered separately or as an admixture, and the frequency of administration of the Compound.

Animal models for cancer can be used to assess the efficacy of a Compound or a combination therapy described herein. Examples of animal models for lung cancer include, but are not limited to, lung cancer animal models described by Zhang & Roth (1994, In Vivo 8(5):755-69) and a transgenic mouse model with disrupted p53 function (see, e.g., Morris et al. J. La. State Med. Soc. 1998, 150(4):179-85). An example of an animal model for breast cancer includes, but is not limited to, a transgenic mouse that overexpresses cyclin D1 (see, e.g., Hosokawa et al., Transgenic Res. 2001, 10(5), 471-8. An example of an animal model for colon cancer includes, but is not limited to, a TCR b and p53 double knockout mouse (see, e.g., Kado et al., Cancer Res. 2001, 61(6):2395-8). Examples of animal models for pancreatic cancer include, but are not limited to, a metastatic model of PancO2 murine pancreatic adenocarcinoma (see, e.g., Wang et al., Int. J. Pancreatol. 2001, 29(1):37-46) and nu-nu mice generated in subcutaneous pancreatic tumours (see, e.g., Ghaneh et al., Gene Ther. 2001, 8(3):199-208). Examples of animal models for non-Hodgkin's lymphoma include, but are not limited to, a severe combined immunodeficiency (“SCID”) mouse (see, e.g., Bryant et al., Lab Invest. 2000, 80(4), 553-73) and an IgHmu-HOX11 transgenic mouse (see, e.g., Hough et al., Proc. Natl. Acad. Sci. U.S.A. 1998, 95(23), 13853-8. An example of an animal model for esophageal cancer includes, but is not limited to, a mouse transgenic for the human papillomavirus type 16 E7 oncogene (see, e.g., Herber et al., J. Virol. 1996, 70(3):1873-81). Examples of animal models for colorectal carcinomas include, but are not limited to, Apc mouse models (see, e.g., Fodde & Smits, Trends Mol. Med. 2001, 7(8):369-73 and Kuraguchi et al., Oncogene 2000, 19(50), 5755-63).

In certain in vivo techniques, an imaging agent, or diagnostic moiety, is used which binds to molecules on cancer cells, e.g., cancer cell surface antigens. For instance, a fluorescent tag, radionuclide, heavy metal, or photon-emitter is attached to an antibody (including an antibody fragment) that binds to a cancer cell surface antigen. The medical practitioner can infuse the labeled antibody into the patient either prior to, during, or following treatment with a Compound, and then the practitioner can place the patient into a total body scanner/developer which can detect the attached label (e.g., fluorescent tag, radionuclide, heavy metal, photon-emitter). The scanner/developer (e.g., CT, MRI, or other scanner, e.g. detector of fluorescent label, that can detect the label) records the presence, amount/quantity, and bodily location of the bound antibody. In this manner, the mapping and quantitation of tag (e.g. fluorescence, radioactivity, etc.) in patterns within a tissue or tissues indicates the treatment efficacy within the patient's body when compared to a reference control such as the same patient at an earlier time point or a patient who has no detectable cancer. For example, a large signal (relative to a reference range or a prior treatment date, or prior to treatment) at a particular location indicates the presence of cancer cells. If this signal is increased relative to a prior date it suggests a worsening of the disease and failure of therapy. Alternatively, a signal decrease indicates that therapy has been effective.

Similarly, in some embodiments, the efficacy of the therapeutic regimen in reducing the amount of cancer cells in animals (including humans) undergoing treatment can be evaluated using in vivo techniques. In one embodiment, the medical practitioner performs the imaging technique with labeled molecule that specifically binds the surface of a cancer cell. In this manner, the mapping and quantitation of tag (e.g., fluorescence, radioactivity) in patterns within a tissue or tissues indicates the treatment efficacy within the body of the patient undergoing treatment.

5.3 Compositions

Provided herein are compositions comprising an effective amount of a Compound and compositions comprising an effective amount of a Compound and a pharmaceutically acceptable carrier or vehicle, wherein a pharmaceutically acceptable carrier or vehicle can comprise an excipient, diluent, or a mixture thereof. In one embodiment, the composition is a pharmaceutical composition. In some embodiments, a pharmaceutical composition described herein is suitable for oral, pulmonary, parenteral, mucosal, transdermal or topical administration. A Compound or composition thereof can also be administered intradermally, intramuscularly, intraperitoneally, percutaneously, intravenously, subcutaneously, intranasally, epidurally, sublingually, intracerebrally, intravaginally, transdermally, rectally, mucosally, intratumorally, by inhalation, or topically to the ears, nose, eyes, or skin. The mode of administration is left to the discretion of the health-care practitioner, and can depend in-part upon the site of the medical condition.

The Compounds can be administered to a patient orally or parenterally in the conventional form of preparations, such as capsules, microcapsules, tablets, granules, powder, troches, pills, suppositories, injections, suspensions and syrups.

Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the Compound in the pharmaceutical composition may be at a level that will exercise the desired effect. In a specific embodiment, the effective amount of a Compound in a pharmaceutical composition is about 0.005 mg/kg of a patient's body weight to about 10 mg/kg of a patient's body weight in unit dosage for both oral and parenteral administration. In another specific embodiment, the effective amount of a Compound in a pharmaceutical composition is about 0.005 mg/kg of a patient's body weight to about 50 mg/kg of a patient's body weight in unit dosage for both oral and parenteral administration. In another specific embodiment, the effective amount of a Compound in a pharmaceutical composition is about 0.005 mg/kg of a patient's body weight to about 100 mg/kg of a patient's body weight in unit dosage for both oral and parenteral administration.

A Compound or composition thereof can be administered once, twice, three, four or more times daily.

A Compound or composition thereof can be administered orally for reasons of convenience. In one embodiment, when administered orally, a Compound or composition thereof is administered with a meal and water. In another embodiment, the Compound is dispersed in water or juice (e.g., apple juice or orange juice) and administered orally as a suspension. In another embodiment, when administered orally, a Compound is administered to a subject in a fasted state.

The compositions comprising Compounds can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry.

Capsules can be prepared by mixing a Compound with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the Compound. Typical diluents include, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the Compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a Compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the Compound can be delayed or prolonged by proper formulation. For example, a slowly soluble pellet of the Compound can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. In certain embodiments, the parenteral preparations can be made long-acting, by dissolving or suspending the Compound in oily or emulsified vehicles that allow it to disperse slowly in the serum.

In one embodiment, provided herein are capsules containing a Compound without an additional carrier, excipient or vehicle.

5.4 Prophylactic and Therapeutic Uses

Presented herein are methods for treating an interferon-sensitive disease. In one aspect, the methods for treating an interferon-sensitive disease involve the administration of a Compound, as a single-agent therapy, to a patient in need thereof. In a specific embodiment, presented herein is a method for treating an interferon-sensitive disease, comprising administering to a patient in need thereof an effective amount of a Compound, as a single agent. In another embodiment, presented herein is a method for treating an interferon-sensitive disease, comprising administering to a patient in need thereof a pharmaceutical composition comprising a Compound, as the single active ingredient, and a pharmaceutically acceptable carrier, excipient or vehicle.

In specific embodiments, provided herein are methods for treating an interferon-sensitive disease, comprising administering to a subject an effective amount of a Compound (e.g., a compound of formula I, II, III, IV, or V).

In another aspect, the methods for treating an interferon-sensitive disease involve the administration of a Compound in combination with another therapy (e.g., one or more additional therapies that do not comprise a Compound, and/or that comprise a different Compound) to a patient in need thereof. Such methods may involve administering a Compound prior to, concurrent with, or subsequent to administration of the additional therapy. In certain embodiments, such methods have an additive or synergistic effect. In a specific embodiment, presented herein is a method for treating an interferon-sensitive disease, comprising administering to a patient in need thereof an effective amount of a Compound and an effective amount of another therapy.

In certain embodiments, the methods for treating an interferon-sensitive disease provided herein increase the survival of a patient diagnosed with an interferon-sensitive disease. In particular embodiments, the methods for treating an interferon-sensitive disease provided herein reduce the mortality of subjects diagnosed with an interferon-sensitive disease. In certain embodiments, the methods for treating an interferon-sensitive disease provided herein increase symptom-free survival of an interferon-sensitive disease patients. In some embodiments, the methods for treating an interferon-sensitive disease provided herein do not cure an interferon-sensitive disease in patients, but prevent the progression or worsening of the disease. In specific embodiments, the methods for treating an interferon-sensitive disease provided herein enhance or improve the therapeutic effect of another therapy. In certain embodiments, the methods for treating an interferon-sensitive disease provided herein prevent the onset or development of an interferon-sensitive disease.

In some aspects, the methods for treating an interferon-sensitive disease provided herein provided herein decrease the requirement for hospitalization. In certain embodiments, the methods for treating an interferon-sensitive disease provided herein provided herein decrease the length and/or frequency of hospitalization.

In specific embodiments, the methods for treating an interferon-sensitive disease provided herein reduce hospitalization (e.g., the frequency or duration of hospitalization) of a patient diagnosed with an interferon-sensitive disease. In some embodiments, the methods for treating an interferon-sensitive disease provided herein reduce hospitalization length of a patient diagnosed with an interferon-sensitive disease. In certain embodiments, the methods for treating an interferon-sensitive disease provided herein decrease the hospitalization rate.

In some aspects, the methods for treating an interferon-sensitive disease provided herein provided herein result in one or more of the following: (i) reduction or prevention of the recurrence of the disease; (ii) reduction of the duration of the disease; (iii) prevention or reduction of organ failure associated with the disease; (iv) elimination or curing of the disease; and/or (v) an improvement in the general quality of life as assessed by, e.g., questionnaire.

In specific embodiments, the methods for treating an interferon-sensitive disease provided herein alleviate or manage one, two or more symptoms associated with an interferon-sensitive disease. Alleviating or managing one, two or more symptoms of an interferon-sensitive disease may be used as a clinical endpoint for efficacy of a Compound for treating an interferon-sensitive disease. In some embodiments, the methods for treating an interferon-sensitive disease provided herein reduce the duration and/or severity of one or more symptoms associated with an interferon-sensitive disease. In some embodiments, the methods for treating an interferon-sensitive disease provided herein prevent or inhibit the onset, progression and/or recurrence of one or more symptoms associated with an interferon-sensitive disease. In some embodiments, the methods for treating an interferon-sensitive disease provided herein reduce the number of symptoms associated with an interferon-sensitive disease. In certain embodiments, the methods for treating an interferon-sensitive disease provided herein inhibit or reduce the progression of one or more symptoms associated therewith.

A Compound or a composition thereof used in a method for treating an interferon-sensitive disease may be used as any line of therapy (e.g., a first, second, third, fourth or fifth line therapy).

In certain embodiments, the methods for treating an interferon-sensitive disease comprise administration of a Compound in an amount sufficient to treat the interferon-sensitive disease.

In certain embodiments of the aforementioned methods, the Compounds, compositions, and pharmaceutical compositions are used in an amount that is not significantly toxic to the cell, tissue, or subject for which it is intended. Methods of testing toxicity include any method known in the art, for example, as described supra and in Section 5.2.2 above. The choice of Compounds to be used depends on a number of factors, including but not limited to the type of interferon-sensitive disease, health and age of the patient, and toxicity or side effects.

In certain embodiments, the interferon-sensitive disease treated in accordance with the methods provided herein is a viral disease (i.e., a disease caused by a virus). The viral disease can be caused by an orthomyxovirus (e.g., influenza virus), Adenovirus, arbovirus, paramyxovirus, baculovirus, coronavirus, papillomavirus, parvovirus, chickenpox virus, reovirus, Ebola virus, Ebola-like virus, echo virus, encephalitis virus, filovirus, hantavirus, hepatitis virus, German measles virus, cytomegalovirus, hemorrhagic fever virus, herpes simplex virus, hepatitis A virus, hepatitis B virus, hepatitis C virus, human immunodeficiency virus (HIV), human papillomavirus (the causative agent of genital warts), human T cell leukemia virus, human T cell lymphoma virus, human T cell lymphotropic virus, Lassa fever virus, Marburg virus, measles virus, mumps virus, myxovirus, nairovirus, nanirnavirus, nariva virus, ndumo virus, Necrovirus, neethling virus, neopvirus, neurotropic virus, Newcastle disease virus, oncornavirus, orbivirus, parainfluenza virus, paramyxovirus, parvovirus, picornavirus, rabies virus, respiratory syncytial virus, rhinovirus, rubella virus, rubeola virus, SARS virus, Sendai virus, simian immunodeficiency virus, simian parainfluenza virus, smallpox virus, varicella zoster virus, variola virus, or a vesicular stomatitis virus (VSV). In a specific embodiment, the Compound used to treat a viral disease is Compound I. In another specific embodiment, the Compound used to treat a viral disease is Compound 13, 26, 27, and/or 28. In another specific embodiment, the Compound used to treat a viral disease is Compound 12, 14, 15, 16, 18, 19, 20, and/or 29.

In certain embodiments, the methods for treating a viral disease with a Compound result in one or more of the following: (i) reduction or prevention of the spread of a virus from one cell to another cell, one tissue to another tissue, or one organ to another organ; (ii) reduction or prevention of the spread of a virus from one subject to another subject; (iii) inhibition or reduction of viral replication; (iv) inhibition or reduction of the entry of a virus into a host cell(s); (v) inhibition or reduction of replication of the viral genome; (vi) inhibition or reduction of synthesis of viral proteins; (vii) inhibition or reduction of assembly of viral particles; (viii) inhibition or reduction of release of viral particles from a host cell(s); and/or (ix) reduction of viral titer. In specific embodiments, the methods for treating a viral disease provided herein reduce or eliminate one, two, or more symptoms associated with the viral disease.

In a specific embodiment, the interferon-sensitive disease is influenza virus disease. Symptoms of influenza virus disease include, but are not limited to, body aches (especially joints and throat), fever, nausea, headaches, irritated eyes, fatigue, sore throat, reddened eyes or skin, and abdominal pain. In a specific embodiment, the Compound used to treat influenza virus disease is Compound 1. In another specific embodiment, the Compound used to treat influenza virus disease is Compound 13, 26, 27, and/or 28. In another specific embodiment, the Compound used to treat influenza virus disease is Compound 12, 14, 15, 16, 18, 19, 20, and/or 29.

In specific embodiments, the methods for treating influenza virus disease provided herein reduce or eliminate one, two, or more of the following: body aches (especially joints and throat), fever, nausea, headaches, irritated eyes, fatigue, sore throat, reddened eyes or skin, and abdominal pain.

In specific embodiments, the methods for treating influenza virus disease provided herein inhibit or reduce replication of the influenza virus. In certain embodiments, the methods for treating influenza virus disease provided herein inhibit or reduce replication of the influenza viral genome, inhibit or reduce the production of viral proteins, and/or reduce viral titers.

In certain embodiments, the interferon-sensitive disease treated in accordance with the methods provided herein is a viral infection. In a specific embodiment, provided herein is a method of treating a virus infection or a symptom associated therewith in a subject, comprising administering to a subject in need thereof an effective amount of a Compound. In a specific embodiment, the Compound used to treat a viral infection is Compound I. In another specific embodiment, the Compound used to treat a viral infection is Compound 13, 26, 27, and/or 28. In another specific embodiment, the Compound used to treat a viral infection is Compound 12, 14, 15, 16, 18, 19, 20, and/or 29.

In certain embodiments, the methods of treating a viral infection prevent the progression of the infection and/or the onset of disease caused by the viral infection. Thus, in some embodiments, a method for preventing the progression of a viral infection and/or the onset of disease caused by the viral infection, comprises administering an effective amount of a Compound to a subject in need thereof. In certain embodiments, the methods of treating a viral infection prevent the onset, progression and/or recurrence of a symptom associated with a viral infection. Thus, in some embodiments, a method for preventing a symptom associated with a viral infection in a subject, comprises administering an effective amount of a Compound to a subject in need thereof.

In a specific embodiment, the interferon-sensitive disease treated in accordance with the methods provided herein is a viral infection, wherein the viral infection is an orthomyxovirus infection. In a specific embodiment, the Compound used to treat an orthomyxovirus infection is Compound 1. In another specific embodiment, the Compound used to treat an orthomyxovirus infection is Compound 13, 26, 27, and/or 28. In another specific embodiment, the Compound used to treat an orthomyxovirus infection is Compound 12, 14, 15, 16, 18, 19, 20, and/or 29. In certain embodiments, the methods of treating an orthomyxovirus infection prevent the progression of the infection and/or the onset of disease caused by the orthomyxovirus infection. Thus, in some embodiments, a method for preventing the progression of an orthomyxovirus infection and/or the onset of disease caused by the orthomyxovirus infection, comprises administering an effective amount of a Compound to a subject in need thereof. In certain embodiments, the methods of treating an orthomyxovirus infection prevent the onset, progression and/or recurrence of a symptom associated with an orthomyxovirus infection. Thus, in some embodiments, a method for preventing a symptom associated with an orthomyxovirus infection in a subject, comprises administering an effective amount of a Compound to a subject in need thereof.

In one embodiment, the interferon-sensitive disease treated in accordance with the methods provided herein is a viral infection, wherein the viral infection is an influenza virus infection. In a specific embodiment, the Compound used to treat an influenza virus infection or a symptom associated therewith is Compound 1. In another specific embodiment, the Compound used to treat an influenza virus infection or a symptom associated therewith is Compound 13, 26, 27, and/or 28. In another specific embodiment, the Compound used to treat an influenza virus infection or a symptom associated therewith is Compound 12, 14, 15, 16, 18, 19, 20, and/or 29. In certain embodiments, the methods of treating an influenza virus infection prevent the progression of the infection and/or the onset of disease caused by the influenza virus infection. Thus, in some embodiments, a method for preventing the progression of an influenza virus infection and/or the onset of disease caused by the influenza virus infection, comprises administering an effective amount of a Compound to a subject in need thereof. In certain embodiments, the methods of treating an influenza virus infection prevent the onset, progression and/or recurrence of a symptom associated with an influenza infection. Thus, in some embodiments, a method for preventing a symptom associated with an influenza virus infection in a subject, comprises administering an effective amount of a Compound to a subject in need thereof.

In another specific embodiment, the interferon-sensitive disease treated in accordance with the methods provided herein is a viral infection, wherein the viral infection is an influenza A, influenza B, or influenza C virus infection. In certain embodiments, the influenza virus is any type, subtype, and/or strain of influenza A virus. In other embodiments, the influenza virus is a particular type, subtype, and/or strain of influenza virus. In a specific embodiment, the Compound used to treat an influenza A, influenza B, or influenza C virus infection is Compound 1. In some embodiments, Compound 1 is used to treat an H1N1, H3N2, and/or H5N1 influenza virus. In another specific embodiment, the Compound used to treat an influenza A, influenza B, or influenza C virus infection is Compound 13, 26, 27, and/or 28. In another specific embodiment, the Compound used to treat an influenza A, influenza B, or influenza C virus infection is Compound 12, 14, 15, 16, 18, 19, 20, and/or 29.

In another embodiment, the interferon-sensitive disease treated in accordance with the methods provided herein is a viral infection, wherein the viral infection is a vesicular stomatitis virus infection. In a specific embodiment, the Compound used to treat a vesicular stomatitis virus infection or a symptom associated therewith is Compound 13, 26, 27, and/or 28. In another specific embodiment, the Compound used to treat a vesicular stomatitis virus infection or a symptom associated therewith is Compound 12, 14, 15, 16, 18, 19, 20, and/or 29.

In another specific embodiment, the interferon-sensitive disease treated in accordance with the methods provided herein is a viral infection, wherein the viral infection is a hepatitis virus (e.g., hepatitis B or hepatitis C) infection. In a specific embodiment, the Compound used to treat a hepatitis virus (e.g., hepatitis B or hepatitis C) infection is Compound 13, 26, 27, and/or 28. In another specific embodiment, the Compound used to treat a hepatitis virus (e.g., hepatitis B or hepatitis C) infection is Compound 12, 14, 15, 16, 18, 19, 20, and/or 29.

In another specific embodiment, the interferon-sensitive disease treated in accordance with the methods provided herein is a viral infection, wherein the viral infection is a retrovirus infection. In a specific embodiment, the Compound used to treat a retrovirus infection is Compound 13, 26, 27, and/or 28. In another specific embodiment, the Compound used to treat a retrovirus infection is Compound 12, 14, 15, 16, 18, 19, 20, and/or 29.

In another specific embodiment, the interferon-sensitive disease treated in accordance with the methods provided herein is a viral infection, wherein the viral infection is a human immunodeficiency virus (HIV) infection. In a specific embodiment, the Compound used to treat a HIV infection is Compound 13, 26, 27, and/or 28. In another specific embodiment, the Compound used to treat a HIV infection is Compound 12, 14, 15, 16, 18, 19, 20, and/or 29.

In a specific embodiment, the interferon-sensitive disease treated in accordance with the methods provided herein is a viral infection, wherein the viral infection is not a retrovirus infection. In a specific embodiment, the interferon-sensitive disease treated in accordance with the methods provided herein is a viral infection, wherein the viral infection is not a HIV infection. In a specific embodiment the interferon-sensitive disease treated in accordance with the methods provided herein is a viral infection, wherein the viral infection is not a hepatitis virus infection. In another specific embodiment, the interferon-sensitive disease treated in accordance with the methods provided herein is not a virus infection. In another specific embodiment, the interferon-sensitive disease treated in accordance with the methods provided herein is a viral infection, wherein the Compound used in the method is not Compound 9. In another specific embodiment, the interferon-sensitive disease treated in accordance with the methods provided herein is an HIV infection, wherein the Compound used in the method is not Compound 4. In another specific embodiment, the interferon-sensitive disease treated in accordance with the methods provided herein is an HIV infection,

wherein the Compound used in the method is not Compound 7. In another specific embodiment, the interferon-sensitive disease treated in accordance with the methods provided herein is an HIV infection, wherein the Compound used in the method is not Compound 10. In certain embodiments, the subject with a viral infection being treated with a Compound does not have cancer.

In certain embodiments, the interferon-sensitive disease treated in accordance with the methods provided herein is cancer. Symptoms of cancer include, but are not limited to, persistent cough or blood-tinged saliva, blood in the stool, anemia, localized pain, blood in the urine, hoarseness, swollen glands, swollen lymph nodes, fever, fatigue, indigestion, difficulty swallowing, reduction in leukocytes, weight loss, itching, sore, headaches, and back pain. In a specific embodiment, the Compound used to treat cancer is Compound 11, 13, 14, 15, 16, 22, 23, 25, 26, 27, and/or 28.

In some embodiments, the methods for treating cancer provided herein reduce or eliminate one, two, or more of the following: persistent cough or blood-tinged saliva, blood in the stool, anemia, localized pain, blood in the urine, hoarseness, swollen glands, swollen lymph nodes, fever, fatigue, indigestion, difficulty swallowing, reduction in leukocytes, weight loss, itching, sore, headaches, and back pain.

In certain embodiments, the methods for treating cancer with a Compound result in one or more of the following: (i) reduction or inhibition of the replication of cancer cells; (ii) an impairment in the formation of a tumor; (iii) eradication, removal, or control of primary, regional and/or metastatic cancer; (iv) the size of a tumor is maintained and does not increase or increases by less than 10%, preferably less than 5%, preferably less than 4%, preferably less than 2% as determined by, e.g., radiology or MRI; (v) an increase in the number of patients in remission from cancer; (vi) a stabilization, reduction or elimination in the cancer cell population; (vii) stabilization or reduction in the growth of a tumor or neoplasm; (viii) a decrease in the need for chemotherapy and/or radiation treatment; (ix) a reduction in hospitalization rate and/or length; (x) an increase in survival; and/or (xi) an increase in cancer-free survival.

In a specific embodiment, the interferon-sensitive disease treated in accordance with the methods provided herein is a hematologic cancer. In a specific embodiment, the Compound used to treat the hematologic cancer is Compound 11, 13, 14, 15, 16, 22, 23, 25, 26, 27, and/or 28.

In another specific embodiment, the interferon-sensitive disease treated in accordance with the methods provided herein is cancer, wherein the cancer the cancer is hairy cell leukemia, chronic myeloid leukemia, nodular lymphoma, cutaneous T-cell lymphoma, papilloma, cutaneous melanoma, or basal cell carcinoma. In a specific embodiment, the Compound used to treat hairy cell leukemia, chronic myeloid leukemia, nodular lymphoma, cutaneous T-cell lymphoma, papilloma, cutaneous melanoma, or basal cell carcinoma is Compound 11, 13, 14, 15, 16, 22, 23, 25, 26, 27, and/or 28.

In a specific embodiment, the interferon-sensitive disease treated in accordance with the methods provided herein a non-hematologic cancer. In a specific embodiment, the Compound used to treat the non-hematologic cancer is Compound 11, 13, 14, 15, 16, 22, 23, 25, 26, 27, and/or 28.

In another specific embodiment, the interferon-sensitive disease treated in accordance with the methods provided herein is cancer, wherein the cancer is not hairy cell leukemia, chronic myeloid leukemia, nodular lymphoma, cutaneous T-cell lymphoma, papilloma, cutaneous melanoma, or basal cell carcinoma. In another specific embodiment, the interferon-sensitive disease treated in accordance with the methods provided herein is not cancer. In another specific embodiment, the interferon-sensitive disease treated in accordance with the methods provided herein is cancer, wherein the Compound used in the method is not Compound 4. In another specific embodiment, the interferon-sensitive disease treated in accordance with the methods provided herein is cancer, wherein the Compound used in the method is not Compound 5. In another specific embodiment, the interferon-sensitive disease treated in accordance with the methods provided herein is cancer, wherein the Compound used in the method is not Compound 6. In another specific embodiment, the interferon-sensitive disease treated in accordance with the methods provided herein is cancer,

wherein the Compound used in the method is not Compound 7. In another specific embodiment, the interferon-sensitive disease treated in accordance with the methods provided herein is cancer, wherein the Compound used in the method is not Compound 9. In another specific embodiment, the interferon-sensitive disease treated in accordance with the methods provided herein is cancer, wherein the Compound used in the method is not Compound 10.

In certain embodiments, the interferon-sensitive disease treated in accordance with the methods provided herein is an autoimmune disease. In a specific embodiment, the Compound used to treat an autoimmune disease is Compound 11, 13, 14, 15, 16, 22, 23, 25, 26, 27, and/or 28. In another specific embodiment, the Compound used to treat the autoimmune disease is Compound 5, 6, 7, 9, and/or 10. In certain embodiments, the subject with an autoimmune disease being treated with a Compound does not have cancer.

In a specific embodiment, the interferon-sensitive disease treated in accordance with the methods provided herein is an autoimmune disease, wherein the autoimmune disease is multiple sclerosis. Symptoms of multiple sclerosis include, but are not limited to, loss of balance, numbness or abnormal sensation in any area, pain associated with muscle spasms, pain in the arms or legs, problems moving arms or legs, problems walking, problems with coordination and making small movements, slurred or difficult-to-understand speech, tremor in one or more arms or legs, uncontrollable spasm of muscle groups (muscle spasticity), weakness in one or more arms or legs, double vision, eye discomfort, uncontrollable rapid eye movements, vision loss, decreased attention span, decreased judgment, decreased memory, depression or feelings of sadness, dizziness, facial pain, hearing loss, fatigue, constipation, difficulty beginning urinating, frequent need to urinate, stool leakage, strong urge to urinate, and urine leakage (incontinence). In a specific embodiment, the Compound used to treat multiple sclerosis is Compound 11, 13, 14, 15, 16, 22, 23, 25, 26, 27, and/or 28. In another specific embodiment, the Compound used to treat multiple sclerosis is Compound 5, 6, 7, 9, and/or 10. In certain embodiments, the subject with multiple sclerosis being treated with a Compound does not have cancer.

In some embodiments, the methods for treating multiple sclerosis provided herein reduce or eliminate one, two, or more of the following: loss of balance, numbness or abnormal sensation in any area, pain associated with muscle spasms, pain in the arms or legs, problems moving arms or legs, problems walking, problems with coordination and making small movements, slurred or difficult-to-understand speech, tremor in one or more arms or legs, uncontrollable spasm of muscle groups (muscle spasticity), weakness in one or more arms or legs, double vision, eye discomfort, uncontrollable rapid eye movements, vision loss, decreased attention span, decreased judgment, decreased memory, depression or feelings of sadness, dizziness, facial pain, hearing loss, fatigue, constipation, difficulty beginning urinating, frequent need to urinate, stool leakage, strong urge to urinate, and urine leakage (incontinence).

In a specific embodiment, the interferon-sensitive disease treated in accordance with the methods provided herein is an autoimmune disease, wherein the autoimmune disease is not multiple sclerosis. In certain embodiments, the interferon-sensitive disease treated in accordance with the methods provided herein is not an autoimmune disease.

In certain embodiments, a subject with an interferon-sensitive disease is treated with a Compound, wherein the interferon-sensitive disease is a bacterial infection or a disease caused by bacteria. Symptoms of bacterial infections include, but are not limited to, localized redness, localized heat, localized, swelling, localized pain, fatigue, dizziness, fever, discharge in the infected area, and general body aches/pain. In a specific embodiment, the Compound used to treat a bacterial infection or a disease caused by bacteria is Compound 11, 13, 14, 15, 16, 22, 23, 25, 26, 27, and/or 28. In another specific embodiment, the Compound used to treat a bacterial infection or a disease caused by bacteria is Compound 5, 6, 7, 9, and/or 10. In certain embodiments, the methods of treating a bacterial infection prevent the progress of the infection and/or the onset of a disease caused by the bacterial infection. Thus, in some embodiments, a method for preventing the progression of a bacterial infection and/or the onset of disease caused by the bacterial infection, comprises administering an effective amount of a Compound to a subject in need thereof. In certain embodiments, the methods of treating a bacterial infection prevent the onset, progression and/or recurrence of a symptom associated with a bacterial infection. Thus, in some embodiments, a method for preventing a symptom associated with a bacteria infection in a subject, comprises administering an effective amount of a Compound to a subject in need thereof. In certain embodiments, the subject with a bacterial infection being treated with a Compound does not have cancer.

In some embodiments, the methods for treating a bacterial infection provided herein reduce or eliminate one, two, or more of the following: localized redness, localized heat, localized, swelling, localized pain, fatigue, dizziness, fever, discharge in the infected area, and/or general body aches/pain.

Also provided herein are methods for enhancing the immune response in a subject to a vaccine (e.g., a live virus vaccine or an inactivated virus vaccine) comprising administering a Compound to a subject receiving or that has received a vaccine, e.g., the Compound is used as an adjuvant. In a specific embodiment, a Compound modulates the effect of the vaccine, e.g., the Compound increases the efficacy of the vaccine. A Compound administered with a vaccine can be administered prior to, concurrently with, or subsequent to the administration of the vaccine. In certain embodiments, the vaccine is a live virus vaccine or other type of attenuated vaccine. In other embodiments, the vaccine is an inactivated virus vaccine or subunit vaccine. In a specific embodiment, the Compound used to enhance the immune response in a subject (e.g., as an adjuvant) is Compound I. In another specific embodiment, the Compound used to enhance the immune response in a subject (e.g., as an adjuvant) is Compound 13, 26, 27, and/or 28. In another specific embodiment, the Compound used to enhance the immune response in a subject (e.g., as an adjuvant) is Compound 12, 14, 15, 16, 18, 19, 20, and/or 29.

5.4.1 Patient Population

In some embodiments, a subject treated for an interferon-sensitive disease in accordance with the methods provided herein is an animal (e.g., a human or non-human animal) that has or is diagnosed with an interferon-sensitive disease. In other embodiments, a subject treated for an interferon-sensitive disease in accordance with the methods provided herein is an animal (e.g., a human or non-human animal) predisposed or susceptible to an interferon-sensitive disease. In some embodiments, a subject treated for an interferon-sensitive disease in accordance with the methods provided herein is an animal (e.g., a human or non-human animal) at risk of developing an interferon-sensitive disease.

In certain embodiments, a Compound is administered to a subject that is to receive a vaccine, i.e., a Compound is used as an adjuvant. In a specific embodiment, the subject receiving the vaccine has an interferon-sensitive disease. In another specific embodiment, the subject receiving the vaccine is at risk of developing an interferon-sensitive disease. In another specific embodiment, the subject receiving the vaccine does not have an interferon-sensitive disease.

In certain embodiments, a Compound is administered to a subject that has received a vaccine. In a specific embodiment, the subject that received the vaccine has an interferon-sensitive disease. In another specific embodiment, the subject that received the vaccine is at risk of developing an interferon-sensitive disease. In another specific embodiment, the subject that received the vaccine does not have an interferon-sensitive disease.

In one embodiment, a subject treated for an interferon-sensitive disease or administered a Compound as an adjuvant in accordance with the methods provided herein is a human infant. In another embodiment, a subject treated for an interferon-sensitive disease or administered a Compound as an adjuvant in accordance with the methods provided herein is a premature human infant. In another embodiment, a subject treated for an interferon-sensitive disease or administered a Compound as an adjuvant in accordance with the methods provided herein is a human toddler. In another embodiment, a subject treated for an interferon-sensitive disease or administered a Compound as an adjuvant in accordance with the methods provided herein is a human child. In another embodiment, a subject treated for an interferon-sensitive disease or administered a Compound as an adjuvant in accordance with the methods provided herein is a human adult. In another embodiment, a subject treated for an interferon-sensitive disease or administered a Compound as an adjuvant in accordance with the methods provided herein is a middle-aged human (e.g., a human aged 30-65 years old). In another embodiment, a subject treated for an interferon-sensitive disease or administered a Compound as an adjuvant in accordance with the methods provided herein is an elderly human.

In certain embodiments, Compound I is administered to patients receiving or that have received a viral vaccine. In a specific embodiment, Compound I is administered to patients receiving or that have received a viral vaccine, wherein the viral vaccine comprises live, attenuated virus. In a specific embodiment, the viral vaccine is influenza vaccine. In a specific embodiment, the viral vaccine is respiratory syncytial virus (RSV) vaccine. In certain other embodiments, the viral vaccine is a rotavirus vaccine, MMR vaccine, yellow fever vaccine, or varicella virus vaccine (e.g., a vaccine for chicken pox or shingles).

In certain embodiments, Compound II is administered to patients receiving or that have received a viral vaccine. In certain embodiments, Compound II is administered to patients receiving or that have received a live viral vaccine. In certain embodiments, Compound II is administered to patients receiving or that have received an inactivated viral vaccine. In certain embodiments, Compound II is administered to patients receiving or that have received an influenza virus vaccine. In certain embodiments, Compound II is administered to patients receiving or that have received an RSV vaccine. In other embodiments, Compound II is administered to patients receiving or that have received a vaccine, wherein the vaccine is not a viral vaccine. In certain embodiments, Compound II is administered to patients receiving or that have received a cancer vaccine.

In certain embodiments, a subject treated for an interferon-sensitive disease or administered a Compound as an adjuvant in accordance with the methods provided herein is a human that is about 1 to about 5 years old, about 5 to 10 years old, about 10 to about 18 years old, about 18 to about 30 years old, about 25 to about 35 years old, about 35 to about 45 years old, about 40 to about 55 years old, about 50 to about 65 years old, about 60 to about 75 years old, about 70 to about 85 years old, about 80 to about 90 years old, about 90 to about 95 years old or about 95 to about 100 years old, or any age in between. In a specific embodiment, a subject treated for an interferon-sensitive disease or administered a Compound an adjuvant in accordance with the methods provided herein is a human that is 18 years old or older. In a certain embodiment, a subject treated for an interferon-sensitive disease or administered a Compound as an adjuvant in accordance with the methods provided herein is a human that is between the age of 12 years old and 18 years old. In a certain embodiment, the subject is a male human. In another embodiment, the subject is a female human. In one embodiment, the subject is a female human that is not pregnant or is not breastfeeding. In one embodiment, the subject is a female that is pregnant or will/might become pregnant, or is breast feeding.

In particular embodiments, a subject treated for an interferon-sensitive disease or administered a Compound as an adjuvant in accordance with the methods provided herein is a human that is in an immunocompromised state or immunosuppressed state. In certain embodiments, a subject treated for an interferon-sensitive disease or administered a Compound as an adjuvant in accordance with the methods provided herein is a human receiving or recovering from immunosuppressive therapy. In certain embodiments, a subject treated for an interferon-sensitive disease or administered a Compound as an adjuvant in accordance with the methods provided herein is a human that has or is at risk of getting cancer (e.g., metastatic cancer), or at risk of being infected by a pathogen. In a specific embodiment, a subject treated for an interferon-sensitive disease or administered a Compound as an adjuvant in accordance with the methods provided herein is a human that has or is at risk of being infected by a virus. In a specific embodiment, a subject treated for an interferon-sensitive disease or administered a Compound as an adjuvant in accordance with the methods provided herein is a human that has or is at risk of being infected by an influenza virus. In a specific embodiment, a subject treated for an interferon-sensitive disease or administered a Compound as an adjuvant in accordance with the methods provided herein is a human that has or is at risk of being infected by a bacteria. In certain embodiments, a subject treated for an interferon-sensitive disease or administered a Compound as an adjuvant in accordance with the methods provided herein is a human who is, will, or has undergone surgery, drug therapy, such as chemotherapy, hormonal therapy and/or radiation therapy.

In specific embodiments, a subject treated for an interferon-sensitive disease in accordance with the methods provided herein is suffering from a condition wherein the administration of therapies other than Compounds may be contraindicated.

In some embodiments, the subject to be treated is severely ill. In some embodiments, the subject to be treated is unresponsive, or poorly responsive, to one or more previous therapies.

In some embodiments, a subject treated for an interferon-sensitive disease in accordance with the methods provided herein is administered a Compound or a pharmaceutical composition thereof, or a combination therapy before any adverse effects or intolerance to therapies other than the Compound develops. In some embodiments, a subject treated for an interferon-sensitive disease in accordance with the methods provided herein is a refractory patient. In a certain embodiment, a refractory patient is a patient refractory to a standard therapy. In certain embodiments, a patient with an interferon-sensitive disease is refractory to a therapy when the interferon-sensitive disease has not significantly been eradicated and/or the one or more symptoms have not been significantly alleviated. The determination of whether a patient is refractory can be made either in vivo or in vitro by any method known in the art for assaying the effectiveness of a treatment of an interferon-sensitive disease, using art-accepted meanings of “refractory” in such a context.

In some embodiments, a subject treated for an interferon-sensitive disease in accordance with the methods provided herein is an animal (e.g., a human or non-human animal) that has proven refractory to therapies other than treatment with a Compound, but is no longer on these therapies. In certain embodiments, a subject treated for an interferon-sensitive disease in accordance with the methods provided herein is an animal (e.g., a human or non-human animal) already receiving one or more conventional therapies used to treat the interferon-sensitive disease. In certain embodiments, a subject treated for an interferon-sensitive disease in accordance with the methods provided herein is an animal (e.g., a human or non-human animal) already receiving one or more conventional anti-cancer therapies, such as surgery, drug therapy such as chemotherapy, anti-androgen therapy or radiation. Among these patients are refractory patients, patients who are too young for conventional therapies, and patients with recurring tumors despite treatment with existing therapies.

In some embodiments, a subject treated for an interferon-sensitive disease in accordance with the methods described herein or administered a Compound as an adjuvant in accordance with the methods provided herein is an animal (e.g., a human or non-human animal) susceptible to adverse reactions to conventional therapies. In some embodiments, a subject treated for an interferon-sensitive disease or administered a Compound as an adjuvant in accordance with the methods provided herein is an animal (e.g., a human or non-human animal) that has not received a therapy, e.g., drug therapy, surgery, or radiation therapy, prior to the administration of a Compound or a pharmaceutical composition thereof. In other embodiments, a subject treated for an interferon-sensitive disease in accordance with the methods provided herein is an animal (e.g., a human or non-human animal) that has received a therapy prior to administration of a Compound. In some embodiments, a subject treated for an interferon-sensitive disease in accordance with the methods provided herein is an animal (e.g., a human or non-human animal) that has experienced adverse side effects to the prior therapy or the prior therapy was discontinued due to unacceptable levels of toxicity to the human.

In certain embodiments, a subject treated for an interferon-sensitive disease in accordance with the methods described herein has cancer. In some embodiments, the subject with cancer has a cancer that has metastasized to other areas of the body, such as the bones, brain, lymph nodes, lung and liver. In some embodiments, the subject is in remission from the cancer. In some embodiments, the subject has a recurrence of the cancer. In some embodiments, the subject is experiencing recurrence of one or more tumors associated with cancer.

In certain embodiments, a subject treated for an interferon-sensitive disease in accordance with the methods described herein has an influenza virus disease or infection. In some embodiments, the influenza virus infection is an active infection. In some embodiments, the influenza virus infection is chronic.

5.4.1.1 Viral Diseases

Viral infections and/or viral diseases that can be treated with the methods provided herein include those caused by the following non-limiting list of viruses: Adenovirus, arbovirus, paramyxovirus, baculovirus, coronavirus, papillomavirus, parvovirus, chickenpox virus, reovirus, Ebola virus, Ebola-like virus, echo virus, encephalitis virus, filovirus, hantavirus, hepatitis virus, German measles virus, cytomegalovirus, hemorrhagic fever virus, herpes simplex virus, human immunodeficiency virus (HIV), human papillomavirus (the causative agent of genital warts), human T cell leukemia virus, human T cell lymphoma virus, human T cell lymphotropic virus, influenza virus, Lassa fever virus, Marburg virus, measles virus, mumps virus, myxovirus, nairovirus, nanirnavirus, nariva virus, ndumo virus, Necrovirus, neethling virus, neopvirus, neurotropic virus, Newcastle disease virus, oncornavirus, orbivirus, orthomyxovirus, parainfluenza virus, paramyxovirus, parvovirus, picornavirus, rabies virus, respiratory syncytial virus, rhinovirus, rubella virus, rubeola virus, SARS virus, Sendai virus, simian immunodeficiency virus, simian parainfluenza virus, smallpox virus, varicella zoster virus, variola virus, and vesicular stomatitis virus (VSV).

5.4.1.1.1 Influenza Viruses

In specific embodiments, the interferon-sensitive disease is caused by an influenza virus. In a specific embodiment, the influenza virus is an influenza A virus. Non-limiting examples of influenza A viruses include subtype H10N4, subtype H10N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9. In specific embodiments, the Compounds presented herein are capable of inhibiting multiple influenza A virus subtypes. In a specific embodiment, the Compound capable of inhibiting multiple influenza A virus subtypes is Compound 1.

Specific examples of strains of influenza A virus include, but are not limited to: A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/1/81 (H1N1); A/sw/Potsdam/1/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d'Armor/3633/84 (H3N2); A/sw/Gent/1/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/Gent/V230/92 (H1N1); A/sw/Leipzig/145/92 (H3N2); A/sw/Re220/92hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/1/97 (H3N2); A/sw/Cote d'Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d'Armor/604/99 (H1N2); A/sw/Cote d'Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d'Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haseliinne/2617/03hp (H1N1); A/sw/Loningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/sw/Visbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/sw/Nortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dötlingen/IDT3780/05 (H1N2); A/sw/Dötlingen/IDT4735/05 (H1N2); A/sw/Egglham/IDT5250/05 (H3N2); A/sw/Harkenblek/IDT4097/05 (H3N2); A/sw/Hertzen/IDT4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-S./IDT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/sw/Voglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); A/wild boar/Germany/R169/2006 (H3N2), rA/Brevig Mission/1/1918 (H1N1), A/Hong Kong/1/1968 (H3N2), A/Udorn/307/1972 (H3N2), A/USSR/90/1977 (H1N1), A/swine/Texas/4199-2/1998 (H3N2), rA/Vietnam/1203/2004 (H5N1), and A/California/04/2009 (H1N1).

Other specific examples of strains of influenza A virus include, but are not limited to: A/Toronto/3141/2009 (H1N1); A/Regensburg/D6/2009 (H1N1); A/Bayern/62/2009 (H1N1); A/Bayern/62/2009 (H1N1); A/Bradenburg/19/2009 (H1N1); A/Bradenburg/20/2009 (H1N1); A/Distrito Federal/2611/2009 (H1N1); A/Mato Grosso/2329/2009 (H1N1); A/Sao Paulo/1454/2009 (H1N1); A/Sao Paulo/2233/2009 (H1N1); A/Stockholm/37/2009 (H1N1); A/Stockholm/41/2009 (H1N1); A/Stockholm/45/2009 (H1N1); A/swine/Alberta/OTH-33-1/2009 (H1N1); A/swine/Alberta/OTH-33-14/2009 (H1N1); A/swine/Alberta/OTH-33-2/2009 (H1N1); A/swine/Alberta/OTH-33-21/2009 (H1N1); A/swine/Alberta/OTH-33-22/2009 (H1N1); A/swine/Alberta/OTH-33-23/2009 (H1N1); A/swine/Alberta/OTH-33-24/2009 (H1N1); A/swine/Alberta/OTH-33-25/2009 (H1N1); A/swine/Alberta/OTH-33-3/2009 (H1N1); A/swine/Alberta/OTH-33-7/2009 (H1N1); A/Beijing/502/2009 (H1N1); A/Firenze/10/2009 (H1N1); A/Hong Kong/2369/2009 (H1N1); A/Italy/85/2009 (H1N1); A/Santo Domingo/572N/2009 (H1N1); A/Catalonia/385/2009 (H1N1); A/Catalonia/386/2009 (H1N1); A/Catalonia/387/2009 (H1N1); A/Catalonia/390/2009 (H1N1); A/Catalonia/394/2009 (H1N1); A/Catalonia/397/2009 (H1N1); A/Catalonia/398/2009 (H1N1); A/Catalonia/399/2009 (H1N1); A/Sao Paulo/2303/2009 (H1N1); A/Akita/1/2009 (H1N1); A/Castro/JXP/2009 (H1N1); A/Fukushima/1/2009 (H1N1); A/Israel/276/2009 (H1N1); A/Israel/277/2009 (H1N1); A/Israel/70/2009 (H1N1); A/Iwate/1/2009 (H1N1); A/Iwate/2/2009 (H1N1); A/Kagoshima/1/2009 (H1N1); A/Osaka/180/2009 (H1N1); A/Puerto Montt/Bio87/2009 (H1N1); A/Sao Paulo/2303/2009 (H N1); A/Sapporo/1/2009 (H N1); A/Stockholm/30/2009 (H N1); A/Stockholm/31/2009 (H N1); A/Stockholm/32/2009 (H N1); A/Stockholm/33/2009 (H N1); A/Stockholm/34/2009 (H1N1); A/Stockholm/35/2009 (H1N1); A/Stockholm/36/2009 (H N1); A/Stockholm/38/2009 (H N1); A/Stockholm/39/2009 (H N1); A/Stockholm/40/2009 (H1N1;) A/Stockholm/42/2009 (H1N1); A/Stockholm/43/2009 (H N1); A/Stockholm/44/2009 (H N1); A/Utsunomiya/2/2009 (H N1); A/WRAIR/0573N/2009 (H N1); and A/Zhejiang/DTID-ZJU01/2009 (H N1).

In other embodiments, the influenza virus is an influenza B virus. Non-limiting examples of influenza B viruses include strain Aichi/5/88, strain Akita/27/2001, strain Akita/5/2001, strain Alaska/16/2000, strain Alaska/1777/2005, strain Argentina/69/2001, strain Arizona/146/2005, strain Arizona/148/2005, strain Bangkok/163/90, strain Bangkok/34/99, strain Bangkok/460/03, strain Bangkok/54/99, strain Barcelona/215/03, strain Beijing/15/84, strain Beijing/184/93, strain Beijing/243/97, strain Beijing/43/75, strain Beijing/5/76, strain Beijing/76/98, strain Belgium/WV106/2002, strain Belgium/WV107/2002, strain Belgium/WV109/2002, strain Belgium/WV114/2002, strain Belgium/WV122/2002, strain Bonn/43, strain Brazil/952/2001, strain Bucharest/795/03, strain Buenos Aires/161/00), strain Buenos Aires/9/95, strain Buenos Aires/SW16/97, strain Buenos Aires/VL518/99, strain Canada/464/2001, strain Canada/464/2002, strain Chaco/366/00, strain Chaco/R113/00, strain Cheju/303/03, strain Chiba/447/98, strain Chongqing/3/2000, strain clinical isolate SA1 Thailand/2002, strain clinical isolate SA10 Thailand/2002, strain clinical isolate SA100 Philippines/2002, strain clinical isolate SA101 Philippines/2002, strain clinical isolate SA110 Philippines/2002), strain clinical isolate SA112 Philippines/2002, strain clinical isolate SA 113 Philippines/2002, strain clinical isolate SA114 Philippines/2002, strain clinical isolate SA2 Thailand/2002, strain clinical isolate SA20 Thailand/2002, strain clinical isolate SA38 Philippines/2002, strain clinical isolate SA39 Thailand/2002, strain clinical isolate SA99 Philippines/2002, strain CNIC/27/2001, strain Colorado/2597/2004, strain Cordoba/VA418/99, strain Czechoslovakia/116/89, strain Czechoslovakia/69/90, strain Daeku/10/97, strain Daeku/45/97, strain Daeku/47/97, strain Daeku/9/97, strain B/Du/4/78, strain B/Durban/39/98, strain Durban/43/98, strain Durban/44/98, strain B/Durban/52/98, strain Durban/55/98, strain Durban/56/98, strain England/1716/2005, strain England/2054/2005), strain England/23/04, strain Finland/154/2002, strain Finland/159/2002, strain Finland/160/2002, strain Finland/161/2002, strain Finland/162/03, strain Finland/162/2002, strain Finland/162/91, strain Finland/164/2003, strain Finland/172/91, strain Finland/173/2003, strain Finland/176/2003, strain Finland/184/91, strain Finland/188/2003, strain Finland/190/2003, strain Finland/220/2003, strain Finland/WV5/2002, strain Fujian/36/82, strain Geneva/5079/03, strain Genoa/11/02, strain Genoa/2/02, strain Genoa/21/02, strain Genova/54/02, strain Genova/55/02, strain Guangdong/05/94, strain Guangdong/08/93, strain Guangdong/5/94, strain Guangdong/55/89, strain Guangdong/8/93, strain Guangzhou/7/97, strain Guangzhou/86/92, strain Guangzhou/87/92, strain Gyeonggi/592/2005, strain Hannover/2/90, strain Harbin/07/94, strain Hawaii/10/2001, strain Hawaii/1990/2004, strain Hawaii/38/2001, strain Hawaii/9/2001, strain Hebei/19/94, strain Hebei/3/94), strain Henan/22/97, strain Hiroshima/23/2001, strain Hong Kong/110/99, strain Hong Kong/1115/2002, strain Hong Kong/1 12/2001, strain Hong Kong/123/2001, strain Hong Kong/1351/2002, strain Hong Kong/1434/2002, strain Hong Kong/147/99, strain Hong Kong/156/99, strain Hong Kong/157/99, strain Hong Kong/22/2001, strain Hong Kong/22/89, strain Hong Kong/336/2001, strain Hong Kong/666/2001, strain Hong Kong/9/89, strain Houston/1/91, strain Houston/1/96, strain Houston/2/96, strain Hunan/4/72, strain Ibaraki/2/85, strain ncheon/297/2005, strain India/3/89, strain India/77276/2001, strain Israel/95/03, strain Israel/WV187/2002, strain Japan/1224/2005, strain Jiangsu/10/03, strain Johannesburg/1/99, strain Johannesburg/96/01, strain Kadoma/1076/99, strain Kadoma/122/99, strain Kagoshima/15/94, strain Kansas/22992/99, strain Khazkov/224/91, strain Kobe/1/2002, strain, strain Kouchi/193/99, strain Lazio/1/02, strain Lee/40, strain Leningrad/129/91, strain Lissabon/2/90), strain Los Angeles/1/02, strain Lusaka/270/99, strain Lyon/1271/96, strain Malaysia/83077/2001, strain Maputo/1/99, strain Mar del Plata/595/99, strain Maryland/1/01, strain Memphis/1/01, strain Memphis/12/97-MA, strain Michigan/22572/99, strain Mie/1/93, strain Milano/1/01, strain Minsk/318/90, strain Moscow/3/03, strain Nagoya/20/99, strain Nanchang/1/00, strain Nashville/107/93, strain Nashville/45/91, strain Nebraska/2/01, strain Netherland/801/90, strain Netherlands/429/98, strain New York/1/2002, strain NIB/48/90, strain Ningxia/45/83, strain Norway/1/84, strain Oman/16299/2001, strain Osaka/1059/97, strain Osaka/983/97-V2, strain Oslo/1329/2002, strain Oslo/1846/2002, strain Panama/45/90, strain Paris/329/90, strain Parma/23/02, strain Perth/211/2001, strain Peru/1364/2004, strain Philippines/5072/2001, strain Pusan/270/99, strain Quebec/173/98, strain Quebec/465/98, strain Quebec/7/01, strain Roma/1/03, strain Saga/S172/99, strain Seoul/13/95, strain Seoul/37/91, strain Shangdong/7/97, strain Shanghai/361/2002), strain Shiga/T30/98, strain Sichuan/379/99, strain Singapore/222/79, strain Spain/WV27/2002, strain Stockholm/10/90, strain Switzerland/5441/90, strain Taiwan/0409/00, strain Taiwan/0722/02, strain Taiwan/97271/2001, strain Tehran/80/02, strain Tokyo/6/98, strain Trieste/28/02, strain Ulan Ude/4/02, strain United Kingdom/34304/99, strain USSR/100/83, strain Victoria/103/89, strain Vienna/1/99, strain Wuhan/356/2000, strain WV194/2002, strain Xuanwu/23/82, strain Yamagata/1311/2003, strain Yamagata/K500/2001, strain Yamagata/16/88, strain Alaska/12/96, strain GA/86, strain NAGASAKI/1/87, strain Tokyo/942/96, and strain Rochester/02/2001.

In other embodiments, the influenza virus is an influenza C virus. Non-limiting examples of influenza C viruses include strain Aichi/1/81, strain Ann Arbor/1/50, strain Aomori/74, strain California/78, strain England/83, strain Greece/79, strain Hiroshima/246/2000, strain Hiroshima/252/2000, strain Hyogo/1/83, strain Johannesburg/66, strain Kanagawa/1/76, strain Kyoto/1/79, strain Mississippi/80, strain Miyagi/1/97, strain Miyagi/5/2000, strain Miyagi/9/96, strain Nara/2/85, strain NewJersey/76, strain pig/Beijing/115/81, strain Saitama/3/2000), strain Shizuoka/79, strain Yamagata/2/98, strain Yamagata/6/2000, strain Yamagata/9/96, strain BERLIN/1/85, strain ENGLAND/892/8, strain GREAT LAKES/1167/54, strain JJ/50, strain PIG/BEIJING/10/81, strain PIG/BEIJING/439/82), strain TAYLOR/1233/47, and strain C/YAMAGATA/10/81.

5.4.1.2 Cancer

In specific embodiments, the interferon-sensitive disease is cancer. Non-limiting examples of cancers that can be treated in accordance with the methods provided herein include: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma (including AIDS-related Kaposi's sarcoma), leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo maligna melanoma, malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Other cancers or other abnormal proliferative diseases that can be treated in accordance with the methods provided herein include, but are not limited to, the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. Cancers associated with aberrations in apoptosis are also included and are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders of the skin, lung, liver, bone, brain, stomach, colon, breast, prostate, bladder, kidney, pancreas, ovary, and/or uterus are encompassed herein.

Non-limiting examples of leukemias and other blood-borne cancers include acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, and hairy cell leukemia.

Non-limiting examples of lymphomas include Hodgkin's disease, non-Hodgkin's Lymphoma, Multiple myeloma, Waldenstrom's macroglobulinemia, Heavy chain disease, and Polycythemia vera.

Non-limiting examples of solid tumors that can be treated in accordance with the methods provided herein include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

5.4.1.3 Other Diseases

In certain embodiments, a Compound is administered to a subject with an interferon-sensitive disease that is not a viral infection and not cancer, e.g., the interferon-sensitive disease is multiple sclerosis. In other embodiments, a Compound is administered to a subject with a pre-malignant condition, e.g., actinic keratosis. In other embodiments, a Compound is administered to a subject with leukocyte adhesion deficiency type I; Peyronie's disease; ulcerative colitis; Crohn's disease; osteoporosis; or chronic hepatitis. Those skilled in the art will understand that the Compounds can be used to treat any disease or condition in which the presence of interferon is beneficial.

5.4.2 μMode of Administration

When administered to a patient, a Compound is preferably administered as a component of a composition that optionally comprises a pharmaceutically acceptable carrier vehicle. The composition can be administered orally, or by any other convenient route, for example, topically, by infusion or bolus injection, by absorption through epithelial or mucocutaneous linings (e.g., oral mucosa, rectal, and intestinal mucosa) and may be administered together with another biologically active agent. Administration can be systemic or local. Various delivery systems are known, e.g., encapsulation in liposomes, microparticles, microcapsules, capsules, and can be used to administer a Compound and pharmaceutically acceptable salts thereof.

Methods of administration include but are not limited to parenteral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of a Compound into the bloodstream.

In specific embodiments, it may be desirable to administer a Compound locally. This may be achieved, for example, and not by way of limitation, by local infusion, topical application, e.g., in conjunction with a wound dressing, by injection, by means of a catheter, by means of a suppository, or by means of an implant, said implant being of a porous, non-porous, or gelatinous material, including membranes, such as sialastic membranes, or fibers.

Pulmonary administration can also be employed, e.g., by use of an inhaler or nebulizer, and formulation with an aerosolizing agent, or via perfusion in a fluorocarbon or synthetic pulmonary surfactant. In certain embodiments, a Compound is formulated as a suppository, with traditional binders and vehicles such as triglycerides.

In specific embodiments, a Compound can be administered topically, ocularly, intranasally or by an inhaler or nebulizer.

In another embodiment, a Compound is delivered in a vesicle, in particular a liposome (See Langer, 1990, Science 249:1527 1533; Treat et al., in Liposomes in the Therapy of Infectious Disease and Bacterial infection, Lopez-Berestein and Fidler (eds.), Liss, New York, pp. 353 365 (1989); Lopez Berestein, ibid., pp. 317 327; See generally ibid.).

In another embodiment, a Compound is delivered in a controlled release system (See, e.g., Goodson, in Medical Applications of Controlled Release, supra, vol. 2, pp. 115 138 (1984)). Examples of controlled-release systems are discussed in the review by Langer, 1990, Science 249:1527 1533 may be used. In one embodiment, a pump may be used (See Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (See Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; See also Levy et al., 1985, Science 228:190; During et al., 1989, Ann. Neurol. 25:351; Howard et al., 1989, J. Neurosurg. 71:105).

5.4.3 Combination Therapies

Presented herein are combination therapies for the treatment of an interferon-sensitive disease which involve the administration of a Compound in combination with one or more additional therapies to a subject in need thereof. In a specific embodiment, presented herein are combination therapies for the treatment of an interferon-sensitive disease which involve the administration of an effective amount of a Compound in combination with an effective amount of another therapy to a subject in need thereof

Therapeutic or prophylactic agents that can be used in combination with a Compound for the treatment of an interferon-sensitive disease include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

Specific examples of agents that can be administered in combination with a Compound include, but are not limited to, vaccines, immunomodulatory agents (e.g., interferon), chemotherapeutic agents, anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, and non-steroidal anti-inflammatory drugs (e.g., aspirin, ibuprofen, diclofenac, and COX-2 inhibitors), pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, and AZT), antibacterial; agents, and antibiotics (e.g., dactinomycin (formerly actinomycin), bleomycin, erythomycin, penicillin, mithramycin, and anthramycin (AMC)). In a specific embodiment, a Compound is administered in combination with a vaccine.

Any therapy which is known to be useful, or which has been used or is currently being used for the prevention, management, and/or treatment of an interferon-sensitive disease or symptom or disease associated therewith can be used in combination with a Compound. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 199 9; Cecil Textbook of Medicine, 20th Ed., Bennett and Plum (eds.), W.B. Saunders, Philadelphia, 1996, and Physicians' Desk Reference (61^{st} ed. 1007) for information regarding therapies (e.g., prophylactic or therapeutic agents) which have been or are currently being used for preventing, treating and/or managing interferon-sensitive diseases.

In some embodiments, the one or more other therapies that are administered in combination with a Compound are administered as supportive measures, such as pain relievers, anti-fever medications, or other therapies that alleviate or assist in the treatment of the interferon-sensitive disease

In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. In specific embodiments, two or more therapies are administered within the same patent visit.

In specific embodiments, the interval of time between the administration of a Compound and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain embodiments, a Compound and one or more additional therapies are administered less than 1 day, 1 week, 2 weeks, 3 weeks, 4 weeks, one month, 2 months, 3 months, 6 months, 1 year, 2 years, or 5 years apart.

In some embodiments, the combination therapies provided herein involve administering a Compound daily, and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain embodiments, a Compound and one or more additional therapies are cyclically administered to a subject. Cycling therapy involves the administration of a Compound for a period of time, followed by the administration of one or more additional therapies for a period of time, and repeating this sequential administration. In certain embodiments, cycling therapy may also include a period of rest where a Compound or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In one embodiment, the number of cycles administered is from 1 to 12 cycles, from 2 to 10 cycles, or from 2 to 8 cycles.

In some embodiments, the methods for treating an interferon-sensitive disease provided herein comprise administering a Compound as a single agent for a period of time prior to administering the Compound in combination with an additional therapy. In certain embodiments, the methods for treating an interferon-sensitive disease provided herein comprise administering an additional therapy alone for a period of time prior to administering a Compound in combination with the additional therapy.

In some embodiments, the administration of a Compound and one or more additional therapies in accordance with the methods presented herein have an additive effect relative the administration of the Compound or said one or more additional therapies alone. In some embodiments, the administration of a Compound and one or more additional therapies in accordance with the methods presented herein have a synergistic effect relative to the administration of the Compound or said one or more additional therapies alone.

The combination of a Compound and one or more additional therapies can be administered to a subject in the same pharmaceutical composition. Alternatively, a Compound and one or more additional therapies can be administered concurrently to a subject in separate pharmaceutical compositions. A Compound and one or more additional therapies can be administered sequentially to a subject in separate pharmaceutical compositions. A Compound and one or more additional therapies may also be administered to a subject by the same or different routes of administration.

The combination therapies provided herein involve administering to a subject to in need thereof a Compound in combination with conventional, or known, therapies for interferon-sensitive diseases. Other therapies for an interferon-sensitive disease or a condition associated therewith are aimed at controlling or relieving one or more symptoms. Accordingly, in some embodiments, the combination therapies provided herein involve administering to a subject to in need thereof a pain reliever, or other therapies aimed at alleviating or controlling one or more symptoms associated with the interferon-sensitive disease or a condition associated therewith.

In some embodiments, a combination therapy comprises the administration of one or more Compounds. In other embodiments, a combination therapy comprises administration of two or more Compounds.

5.4.3.1 Antiviral Agents

Antiviral agents that can be used in combination with a Compound include, but are not limited to, non-nucleoside reverse transcriptase inhibitors, nucleoside reverse transcriptase inhibitors, protease inhibitors, and fusion inhibitors. In one embodiment, the antiviral agent is selected from the group consisting of amantadine, oseltamivir phosphate, rimantadine, and zanamivir. In another embodiment, the antiviral agent is a non-nucleoside reverse transcriptase inhibitor selected from the group consisting of delavirdine, efavirenz, and nevirapine. In another embodiment, the antiviral agent is a nucleoside reverse transcriptase inhibitor selected from the group consisting of abacavir, didanosine, emtricitabine, emtricitabine, lamivudine, stavudine, tenofovir DF, zalcitabine, and zidovudine. In another embodiment, the antiviral agent is a protease inhibitor selected from the group consisting of amprenavir, atazanavir, fosamprenav, indinavir, lopinavir, nelfinavir, ritonavir, and saquinavir. In another embodiment, the antiviral agent is a fusion inhibitor such as enfuvirtide.

Additional, non-limiting examples of antiviral agents for use in combination with a Compound include the following: rifampicin, nucleoside reverse transcriptase inhibitors (e.g., AZT, ddI, ddC, 3TC, d4T), non-nucleoside reverse transcriptase inhibitors (e.g., delavirdine efavirenz, nevirapine), protease inhibitors (e.g., aprenavir, indinavir, ritonavir, and saquinavir), idoxuridine, cidofovir, acyclovir, ganciclovir, zanamivir, amantadine, and palivizumab. Other examples of anti-viral agents include but are not limited to acemannan; acyclovir; acyclovir sodium; adefovir; alovudine; alvircept sudotox; amantadine hydrochloride (SYMMETREL™); aranotin; arildone; atevirdine mesylate; pyridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscamet sodium; fosfonet sodium; ganciclovir; ganciclovir sodium; idoxuridine; kethoxal; lamivudine; lobucavir; memotine hydrochloride; methisazone; nevirapine; oseltamivir phosphate (TAMIFLU™); penciclovir; pirodavir; ribavirin; rimantadine hydrochloride (FLUMADINE™); saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zanamivir (RELENZA™); zidovudine; and zinviroxime.

In specific embodiments, the anti-viral agent is an immunomodulatory agent that is specific for a viral antigen. In particular embodiments, the viral antigen is an influenza virus polypeptide other than a hemagglutinin polypeptide. In other embodiments, the viral antigen is an influenza virus hemagglutinin polypeptide.

5.4.3.2 Antibacterial Agents

Antibacterial agents, including antibiotics, that can be used in combination with a Compound include, but are not limited to, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof. In some embodiments, antibiotics are administered in combination with a Compound to prevent and/or treat a bacterial infection.

In a specific embodiment, Compounds are used in combination with other protein synthesis inhibitors, including but not limited to, streptomycin, neomycin, erythromycin, carbomycin, and spiramycin.

In one embodiment, the antibacterial agent is selected from the group consisting of ampicillin, amoxicillin, ciprofloxacin, gentamycin, kanamycin, neomycin, penicillin G, streptomycin, sulfanilamide, and vancomycin. In another embodiment, the antibacterial agent is selected from the group consisting of azithromycin, cefonicid, cefotetan, cephalothin, cephamycin, chlortetracycline, clarithromycin, clindamycin, cycloserine, dalfopristin, doxycycline, erythromycin, linezolid, mupirocin, oxytetracycline, quinupristin, rifampin, spectinomycin, and trimethoprim.

Additional, non-limiting examples of antibacterial agents for use in combination with a Compound include the following: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams (e.g., aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, penamacillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2,4 diaminopyrimidines (e.g., brodimoprim).

5.4.3.3 Anticancer Agents

Specific examples of anti-cancer agents that may be used in combination with a Compound include: hormonal agents (e.g., aromatase inhibitor, selective estrogen receptor modulator (SERM), and estrogen receptor antagonist), chemotherapeutic agents (e.g., microtubule dissembly blocker, antimetabolite, topisomerase inhibitor, and DNA crosslinker or damaging agent), anti-angiogenic agents (e.g., VEGF antagonist, receptor antagonist, integrin antagonist, vascular targeting agent (VTA)/vascular disrupting agent (VDA)), radiation therapy, and conventional surgery.

Non-limiting examples of hormonal agents that may be used in combination with a Compound include aromatase inhibitors, SERMs, and estrogen receptor antagonists. Hormonal agents that are aromatase inhibitors may be steroidal or nonsteroidal. Non-limiting examples of nonsteroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as Nolvadex®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapeutic agents that may be used in combination with a Compound include microtubule disassembly blocker, antimetabolite, topisomerase inhibitor, and DNA crosslinker or damaging agent. Chemotherapeutic agents that are microtubule disassemby blockers include, but are not limited to, taxenes (e.g., paclitaxel (branded/marketed as TAXOL®), docetaxel, abraxane, larotaxel, ortataxel, and tesetaxel); epothilones (e.g., ixabepilone); and vinca alkaloids (e.g., vinorelbine, vinblastine, vindesine, and vincristine (branded/marketed as ONCOVIN®)).

Chemotherapeutic agents that are antimetabolites include, but are not limited to, folate anitmetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine (GEMZAR®), cytarabine, decitabine, floxuridine, tegafur); and deoxyribonucleotide antimetabolites (e.g., hydroxyurea).

Chemotherapeutic agents that are topoisomerase inhibitors include, but are not limited to, class I (camptotheca) topoisomerase inhibitors (e.g., topotecan (branded/marketed as HYCAMTIN®) irinotecan, rubitecan, and belotecan); class II (podophyllum) topoisomerase inhibitors (e.g., etoposide or VP-16, and teniposide); anthracyclines (e.g., doxorubicin, epirubicin, Doxil, aclarubicin, amrubicin, daunorubicin, idarubicin, pirarubicin, valrubicin, and zorubicin); and anthracenediones (e.g., mitoxantrone, and pixantrone).

Chemotherapeutic agents that are DNA crosslinkers (or DNA damaging agents) include, but are not limited to, alkylating agents (e.g., cyclophosphamide, mechlorethamine, ifosfamide (branded/marketed as IFEX®), trofosfamide, chlorambucil, melphalan, prednimustine, bendamustine, uramustine, estramustine, carmustine (branded/marketed as BiCNU®), lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin (branded/marketed as PARAPLATIN®), cisplatin, oxaliplatin, nedaplatin, triplatin tetranitrate, satraplatin, picoplatin); nonclassical DNA crosslinkers (e.g., procarbazine, dacarbazine, temozolomide (branded/marketed as TEMODAR®), altretamine, mitobronitol); and intercalating agents (e.g., actinomycin, bleomycin, mitomycin, and plicamycin).

5.4.3.1 Vaccines

Any vaccine can be used in combination with a Compound in the compositions and methods described herein. A non-limiting list of vaccines includes Anthrax vaccine, BCG vaccine, Diphtheria vaccine, tetanus vaccine, pertussis vaccine, haemophilus B vaccine, hepatitis A vaccine, hepatitis B vaccine, human papillomavirus vaccine, influenza vaccine, Japanese encephalitis virus vaccine, measles vaccine, mumps vaccine, plague vaccine, pneumococcal vaccine, poliovirus vaccine, rabies vaccine, respiratory syncytial virus (RSV) vaccine, rotavirus vaccine, rubella vaccine, smallpox vaccine, typhoid vaccine, varicella virus vaccine, yellow fever vaccine, and zoster vaccine.

5.4.4 Dosage and Frequency of Administration

The amount of a Compound, or the amount of a composition comprising a Compound, that will be effective in the treatment of an interferon-sensitive disease can be determined by standard clinical techniques. In vitro or in vivo assays may optionally be employed to help identify optimal dosage ranges. The precise dose to be employed will also depend, e.g., on the route of administration, the type of interferon-sensitive disease, and the seriousness of the interferon-sensitive disease, and should be decided according to the judgment of the practitioner and each patient's or subject's circumstances.

In some embodiments, the dosage of a Compound is determined by extrapolating from the “no observed adverse effective level” (NOAEL), as determined in animal studies. This extrapolated dosage is useful in determining the maximum recommended starting dose for human clinical trials. For instance, the NOAELs can be extrapolated to determine human equivalent dosages (HED). Typically, HED is extrapolated from a non-human animal dosage based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, baboons), micropigs or minipigs. For a discussion on the use of NOAELs and their extrapolation to determine human equivalent doses, See Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005. In one embodiment, a Compound or composition thereof is administered at a dose that is lower than the human equivalent dosage (HED) of the NOAEL over a period of 1 week, 2 weeks, 3 weeks, 1 month, 2 months, three months, four months, six months, nine months, 1 year, 2 years, 3 years, 4 years or more.

In certain embodiments, a dosage regime for a human subject can be extrapolated from animal model studies using the dose at which 10% of the animals die (LD₁₀). In general the starting dose of a Phase I clinical trial is based on preclinical testing. A standard measure of toxicity of a drug in preclinical testing is the percentage of animals that die because of treatment. It is well within the skill of the art to correlate the LD₁₀ in an animal study with the maximal-tolerated dose (MTD) in humans, adjusted for body surface area, as a basis to extrapolate a starting human dose. In some embodiments, the interrelationship of dosages for one animal model can be converted for use in another animal, including humans, using conversion factors (based on milligrams per meter squared of body surface) as described, e.g., in Freireich et al., Cancer Chemother. Rep., 1966, 50:219-244. Body surface area may be approximately determined from height and weight of the patient. See, e.g., Scientific Tables, Geigy Pharmaceuticals, Ardley, N.Y., 1970, 537. In certain embodiments, the adjustment for body surface area includes host factors such as, for example, surface area, weight, metabolism, tissue distribution, absorption rate, and excretion rate. In addition, the route of administration, excipient usage, and the specific influenza virus or symptom thereof (and/or other disease) to target are also factors to consider. In one embodiment, the standard conservative starting dose is about 1/10 the murine LD₁₀, although it may be even lower if other species (i.e., dogs) were more sensitive to the Compound. In other embodiments, the standard conservative starting dose is about 1/100, 1/95, 1/90, 1/85, 1/80, 1/75, 1/70, 1/65, 1/60, 1/55, 1/50, 1/45, 1/40, 1/35, 1/30, 1/25, 1/20, 1/15, 2/10, 3/10, 4/10, or 5/10 of the murine LD₁₀. In other embodiments, a starting dose amount of a Compound in a human is lower than the dose extrapolated from animal model studies. In another embodiment, a starting dose amount of a Compound in a human is higher than the dose extrapolated from animal model studies. It is well within the skill of the art to start doses of the active composition at relatively low levels, and increase or decrease the dosage as necessary to achieve the desired effect with minimal toxicity.

In certain embodiments, the Compounds can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight or about 0.25 mg/kg of a patient's body weight to about 0.5 mg/kg of a patient's body weight. In one embodiment, one dose is given per day. In any given case, the amount of the Compound administered will depend on such factors as the solubility of the active component, the formulation used and the route of administration.

In another embodiment, provided herein are methods for the treatment of an interferon-sensitive disease comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a Compound to a patient in need thereof.

In another embodiment, provided herein are methods for the treatment of an interferon-sensitive disease comprising the administration of about 1 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 400 mg/day to about 800 mg/day or about 600 mg/day to about 800 mg/day of a Compound to a patient in need thereof. In a particular embodiment, the methods disclosed herein comprise the administration of 400 mg/day, 600 mg/day or 800 mg/day of a Compound to a patient in need thereof.

In another embodiment, provided herein are unit dosage formulations that comprise between about 1 mg and about 2000 mg, about 1 mg and 200 mg, about 35 mg and about 1400 mg, about 125 mg and about 1000 mg, about 250 mg and about 1000 mg, or about 500 mg and about 1000 mg of a Compound.

In a particular embodiment, provided herein are unit dosage formulation comprising about 100 mg or 400 mg of a Compound.

In another embodiment, provided herein are unit dosage formulations that comprise 1 mg, 2.5 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a Compound.

In certain embodiments, suitable dosage ranges for oral administration are about 0.001 milligram to about 500 milligrams of a Compound, per kilogram body weight per day. In specific embodiments, the oral dose is about 0.01 milligram to about 100 milligrams per kilogram body weight per day, about 0.1 milligram to about 75 milligrams per kilogram body weight per day or about 0.5 milligram to 5 milligrams per kilogram body weight per day. The dosage amounts described herein refer to total amounts administered; that is, if more than one Compound is administered, then, in some embodiments, the dosages correspond to the total amount administered. In a specific embodiment, oral compositions contain about 10% to about 95% of a Compound by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. In some embodiments, suitable dosage ranges for intranasal administration are about 0.01 pg/kg body weight per day to about 1 mg/kg body weight per day. Suppositories generally contain about 0.01 milligram to about 50 milligrams of a Compound per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 500 milligrams per kilogram of body weight per day. Suitable doses for topical administration include doses that are in the range of about 0.001 milligram to about 50 milligrams, depending on the area of administration. Effective doses may be extrapolated from dose-response curves derived from in vitro or animal model test systems. Such animal models and systems are well known in the art.

In another embodiment, a subject is administered one or more doses of a prophylactically or therapeutically effective amount of a Compound or a composition described herein, wherein the effective amount is not the same for each dose.

In certain embodiments, wherein the interferon-sensitive disease in a subject is a viral infection, the subject is administered a Compound or a composition described herein in an amount effective to inhibit viral genome replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a Compound or a composition in an amount effective to inhibit or reduce viral genome replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In certain embodiments, a subject is administered a Compound or a composition in an amount effective to inhibit or reduce viral genome replication by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or other known to one of skill in the art.

In certain embodiments, wherein the interferon-sensitive disease in a subject is a viral infection, the subject is administered a Compound or a composition described herein in an amount effective to inhibit or reduce viral protein synthesis by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a Compound or a composition in an amount effective to inhibit or reduce viral protein synthesis by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In certain embodiments, a subject is administered a Compound or a composition in an amount effective to inhibit or reduce viral protein synthesis by at least 1.5 fold, 2 fold, 2.5 fold, 3 fold, 4 fold, 5 fold, 8 fold, 10 fold, 15 fold, 20 fold, or 2 to 5 fold, 2 to fold, 5 to 10 fold, or 5 to 20 fold relative to a negative control as determined using an assay described herein or others known to one of skill in the art.

In certain embodiments, wherein the interferon-sensitive disease in a subject is a viral infection, the subject is administered a Compound or a composition described herein in an amount effective to inhibit or reduce the spread of virus from a cell, tissue, or organ to another cell, tissue or organ by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, a subject is administered a Compound or a composition in an amount effective to inhibit or reduce the spread of virus from a cell, tissue or organ to another cell, tissue or organ by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

In certain embodiments, wherein the interferon-sensitive disease in a subject is a viral infection, the subject is administered a Compound or a composition described herein in an amount effective to inhibit or reduce viral titer by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, a subject is administered a Compound or a composition in an amount effective to inhibit or reduce viral titer by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art. In other embodiments, a subject is administered a Compound or a composition in an amount effective to inhibit or reduce viral titer by 1 log, 1.5 logs, 2 logs, 2.5 logs, 3 logs, 3.5 logs, 4 logs, 5 logs or more relative to a negative control as determined using an assay described herein or others known to one of skill in the art.

In certain embodiments, wherein the interferon-sensitive disease in a subject is a viral infection, the subject is administered a Compound or a composition described herein in an amount effective to inhibit or reduce viral replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, a subject is administered a Compound or a composition in an amount effective to inhibit or reduce viral replication by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or to 20 fold relative to a negative control as determined using an assay described herein or others known to one of skill in the art. In other embodiments, a subject is administered a Compound or a composition described herein in an amount effective to inhibit or reduce viral replication by 1 log, 1.5 logs, 2 logs, 2.5 logs, 3 logs, 3.5 logs, 4 logs, 5 logs or more relative to a negative control as determined using an assay described herein or others known to one of skill in the art.

In certain embodiments, wherein the interferon-sensitive disease in a subject is a viral infection, the subject is administered a Compound or a composition described herein in an amount effective to inhibit or reduce the ability of the virus to spread to other individuals by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a Compound or a composition in an amount effective to inhibit or reduce the ability of the virus to spread to other cells, tissues or organs in the subject by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art.

In certain embodiments, wherein the interferon-sensitive disease in a subject is cancer, the subject is administered a Compound or a composition described herein in an amount effective to inhibit cancer cell replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In certain embodiments, a subject is administered a Compound or a composition in an amount effective to inhibit cancer cell replication by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or other known to one of skill in the art. In certain embodiments, wherein the interferon-sensitive disease in a subject is cancer, the subject is administered a Compound or a composition described herein in an amount effective to reduce or stabilize the size of a tumor. In certain embodiments, wherein the interferon-sensitive disease in a subject is cancer, the subject is administered a Compound or a composition described herein in an amount effective to prevent the spread of the cancer (i.e., metastasis) in the subject.

In certain embodiments, a dose of a Compound or a composition described herein is administered to a subject every day, every other day, every couple of days, every third day, once a week, twice a week, three times a week, or once every two weeks. In other embodiments, two, three or four doses of a Compound or a composition described herein is administered to a subject every day, every couple of days, every third day, once a week or once every two weeks. In some embodiments, a dose(s) of a Compound or a composition is administered for 2 days, 3 days, 5 days, 7 days, 14 days, or 21 days. In certain embodiments, a dose of a Compound or a composition described herein is administered for 1 month, 1.5 months, 2 months, 2.5 months, 3 months, 4 months, 5 months, 6 months or more.

The dosages of prophylactic or therapeutic agents which have been or are currently used for the prevention, treatment and/or management of an interferon-sensitive disease can be determined using references available to a clinician such as, e.g., the Physicians' Desk Reference (64th ed. 2010). In a specific embodiment, dosages lower than those which have been or are currently being used to prevent, treat and/or manage the interferon-sensitive disease are utilized in combination with one or more Compounds or compositions described herein.

For Compounds which have already been approved for uses other than treatment of interferon-sensitive diseases, safe ranges of doses can be readily determined using references available to clinicians, such as e.g., the Physician's Desk Reference (61^{st} ed. 2007).

The above-described administration schedules are provided for illustrative purposes only and should not be considered limiting. A person of ordinary skill in the art will readily understand that all doses are within the scope of the embodiments described herein.

5.5 Cell Culture Uses

In certain embodiments, a Compound is used to increase interferon production in cultured cells. For example, a Compound may be added to cultured cells to induce interferon production by the cultured cells. In specific embodiments, a compound is added to cultured cells to induce production of recombinant interferon by the cells. In certain embodiments, the interferon produced by the cultured cells is collected and used for a secondary application, such as for a treatment of a condition recited herein. In certain embodiments, the interferon produced by the cells is isolated/purified.

In certain embodiments, a Compound is used to increase interferon production in cells cultured in a bioreactor. In specific embodiments, the interferon produced by the cells is recombinant interferon. In certain embodiments, the interferon is collected and used for a secondary application, such as for a treatment of a condition recited herein. In certain embodiments, the interferon produced by the cells is isolated/purified.

5.6 Kits

Provided herein are pharmaceutical kits comprising one or more containers filled with a Compound or a pharmaceutical composition thereof. Also provided herein are pharmaceutical kits comprising one or more containers filled with a Compound or a pharmaceutical composition thereof and one or more containers filled with one or more of the ingredients of the pharmaceutical compositions described herein. Additionally, instructions for the use (e.g., relating to the dosage amount, dosing frequency or route(s) of administration) can also be included in the pharmaceutical kit. Optionally associated with such kits can be a notice in the form prescribed by a governmental agency regulating the manufacture, use or sale of pharmaceuticals or biological products, which notice reflects approval by the agency of manufacture, use or sale for human administration.

Further provided herein are kits for use in a research or laboratory setting, comprising one or more containers filled with a Compound. Such kits can further comprise instructions for carrying out a laboratory experiment or procedure, such as that wherein the Compound(s) is used as a standard or control. Such kits can further comprise one or more containers filled with additional reagents useful or needed for carrying out a laboratory experiment or procedure. Such kits can further comprise cells that express a reporter gene (e.g., a luciferase reporter gene) operably linked to an interferon promoter. Such kits can further comprise a vector that expresses a reporter gene (e.g., a luciferase reporter gene) operably linked to an interferon promoter gene.

### OTHER EMBODIMENTS

In one embodiment, provided herein is a method of inhibiting or reducing replication of an influenza virus in a subject, comprising administering to a subject in need thereof an effective amount of a Compound, wherein the Compound has the structure:

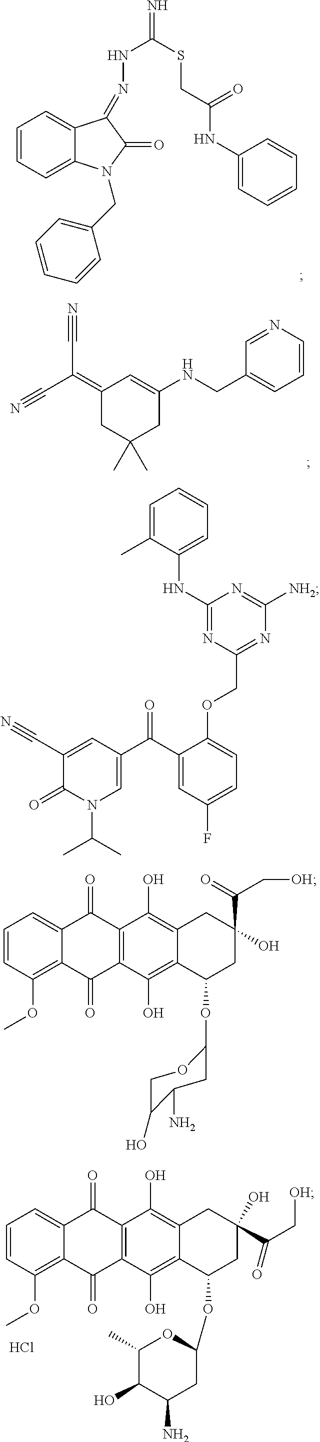

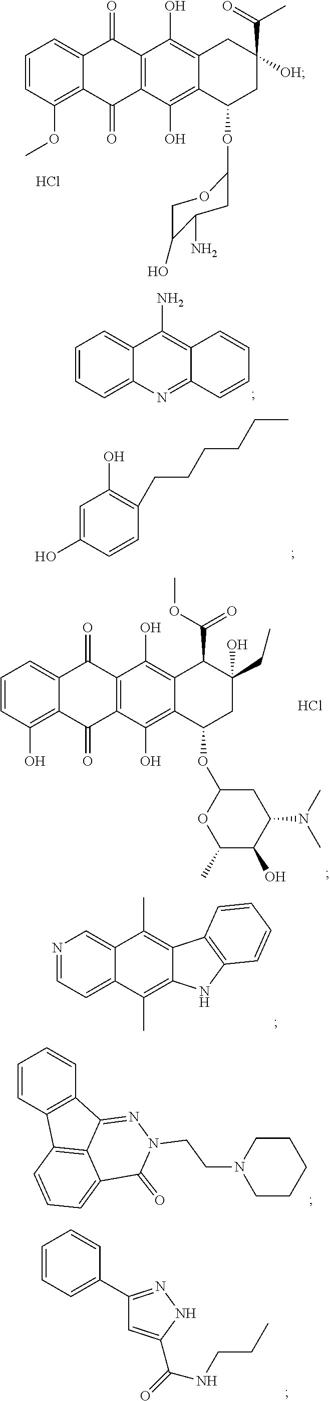

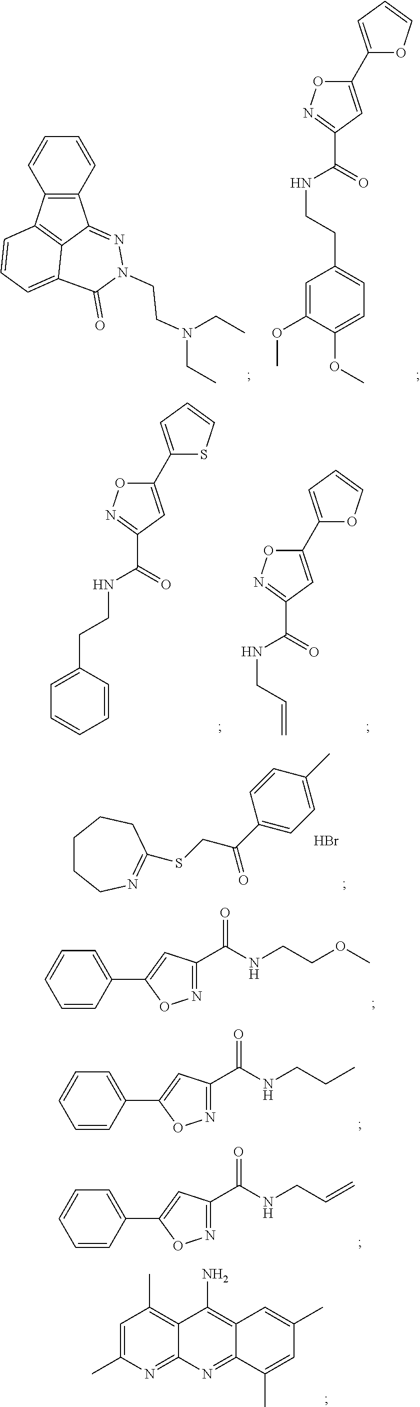

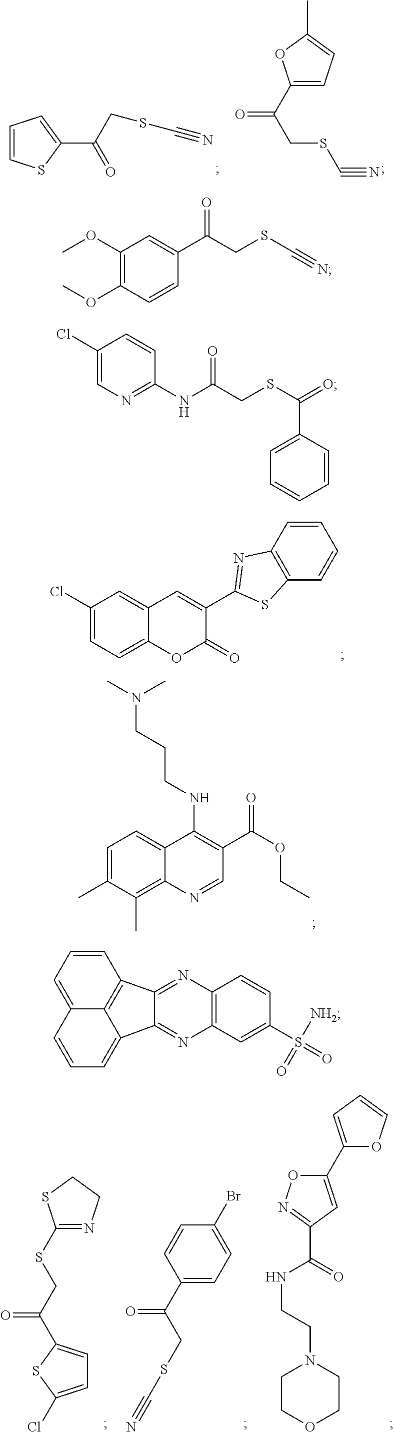

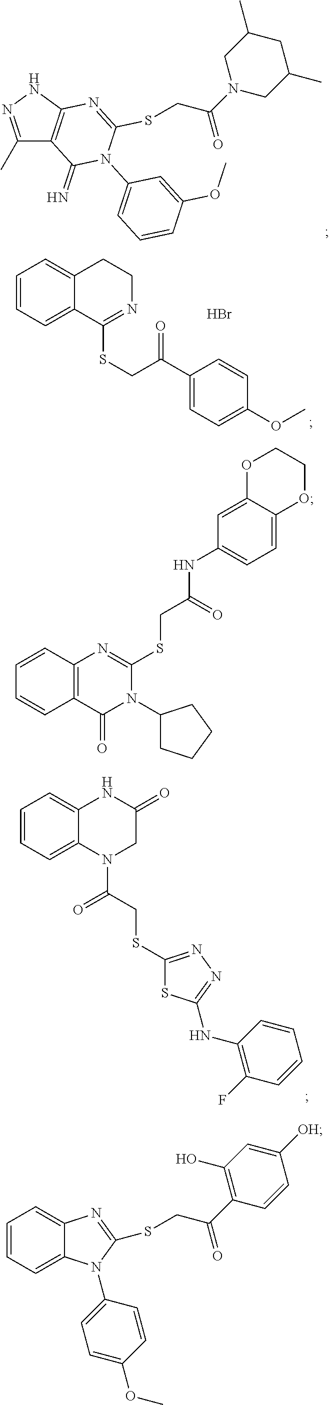

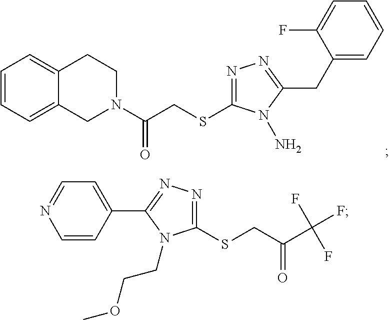

or a pharmaceutically acceptable salt or stereoisomer, including enantiomer, diastereomer, racemate or mixtures of stereoisomer, thereof. In one embodiment, the subject is a human.

In another embodiment, provided herein is a method of treating an influenza virus infection or a symptom associated therewith in a subject, comprising administering to a subject in need thereof an effective amount of a Compound, wherein the Compound has the structure:

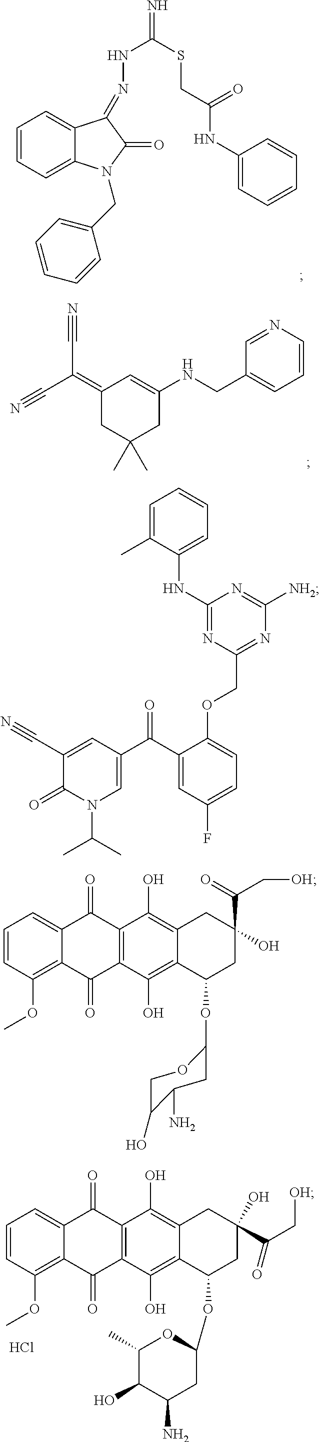

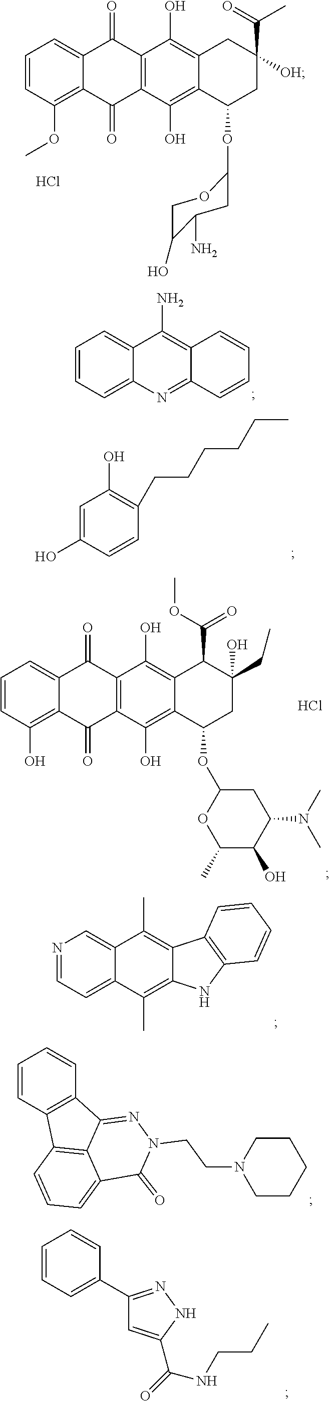

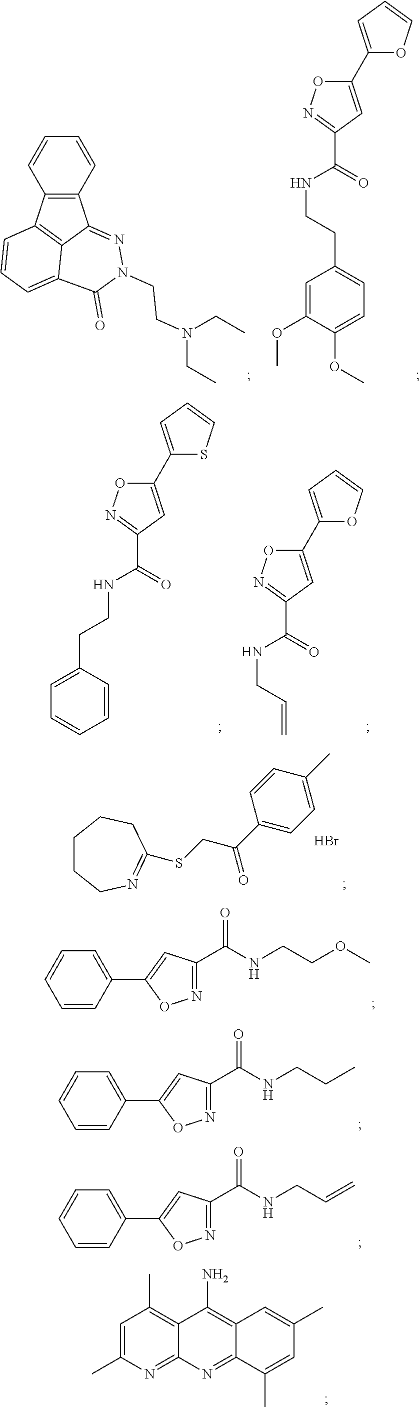

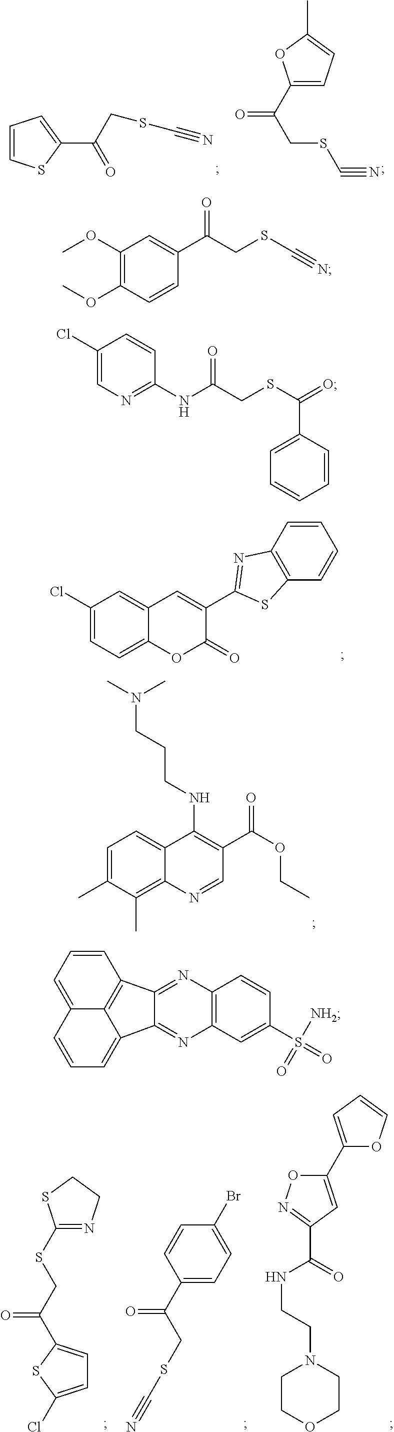

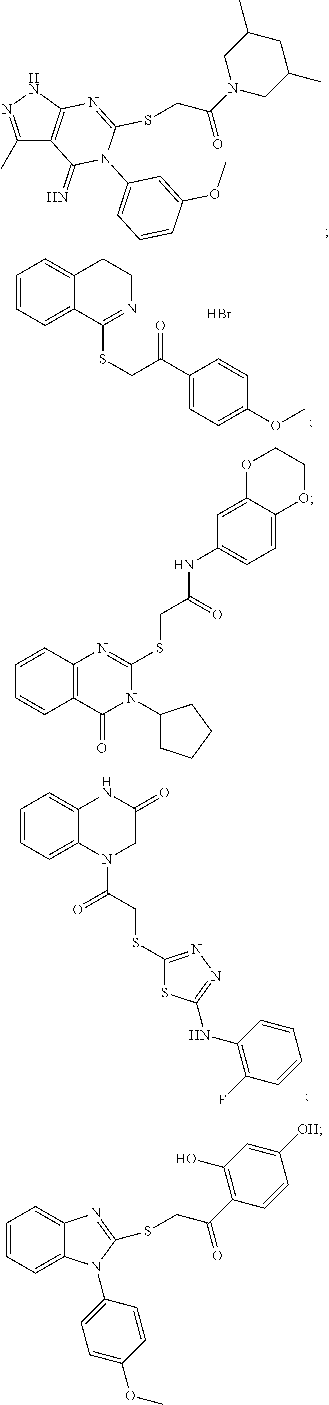

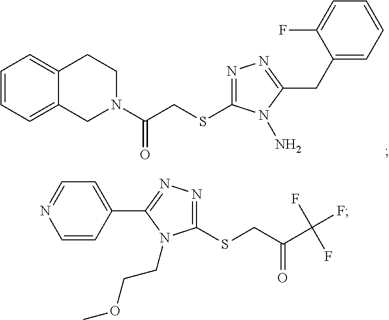

or a pharmaceutically acceptable salt or stereoisomer, including enantiomer, diastereomer, racemate or mixtures of stereoisomer, thereof. In one embodiment, the subject is a human.

In another embodiment, provided herein is a method of preventing a symptom associated with an influenza virus infection in a subject, comprising administering to a subject in need thereof an effective amount of a Compound, wherein the Compound has the structure:

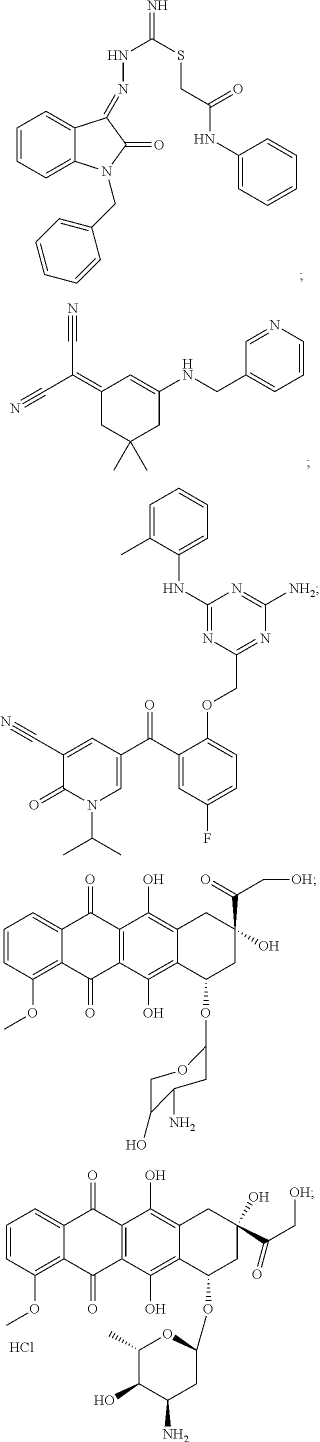

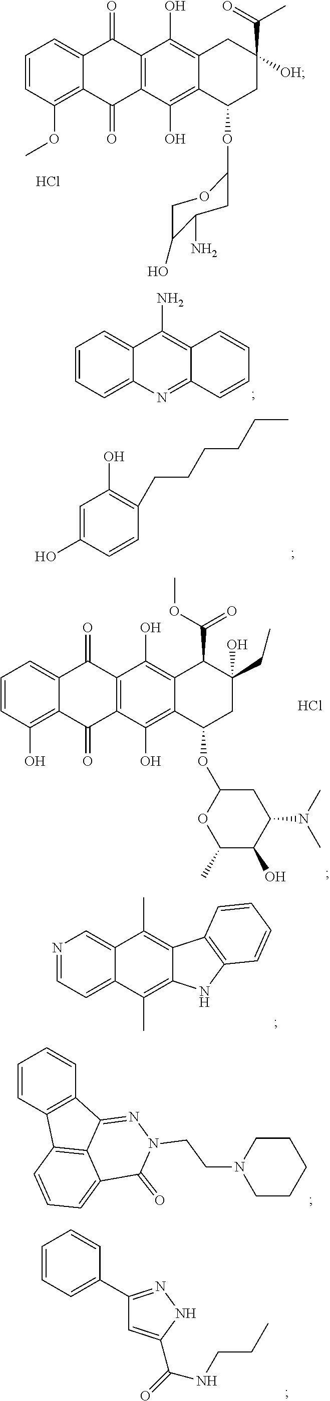

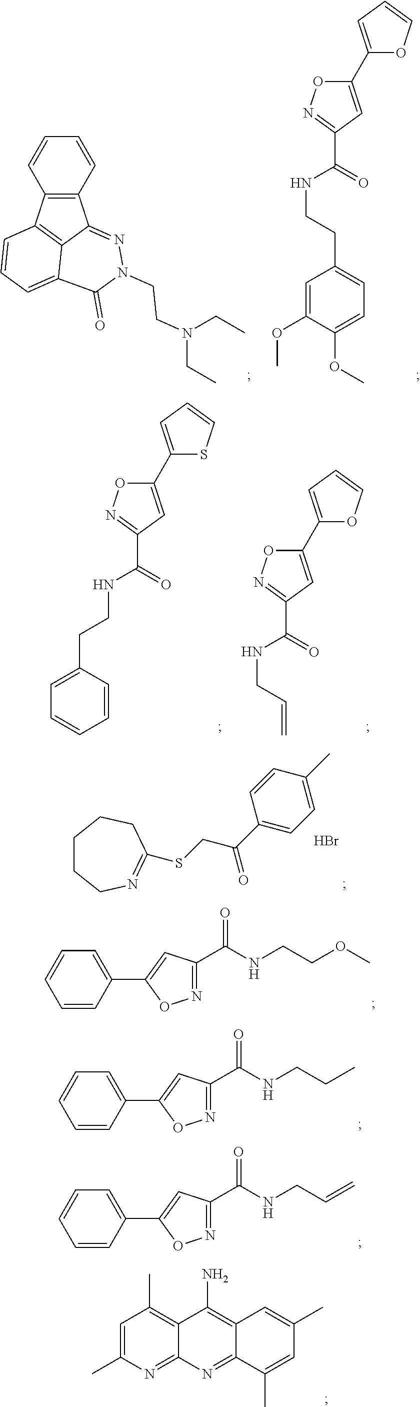

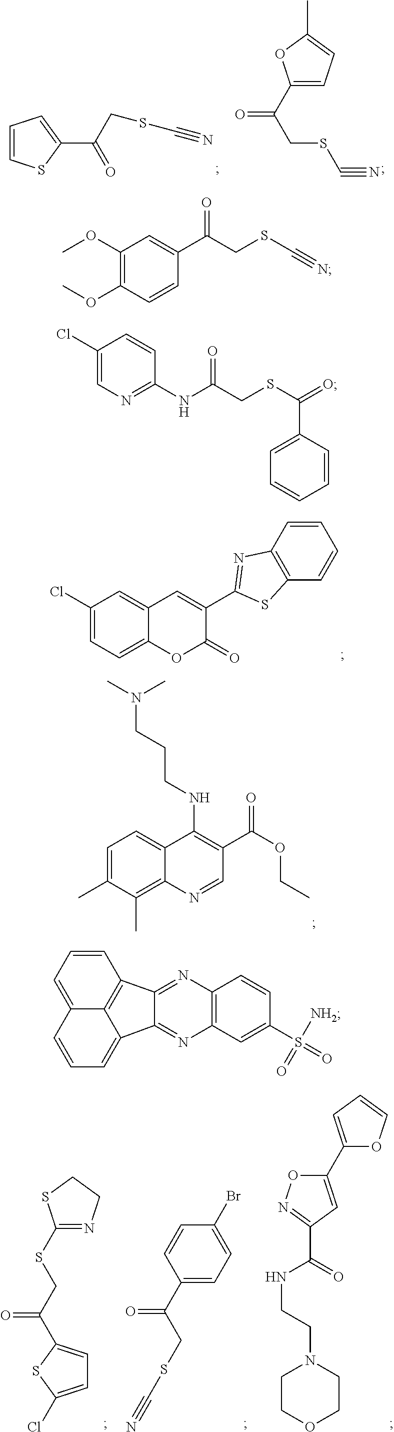

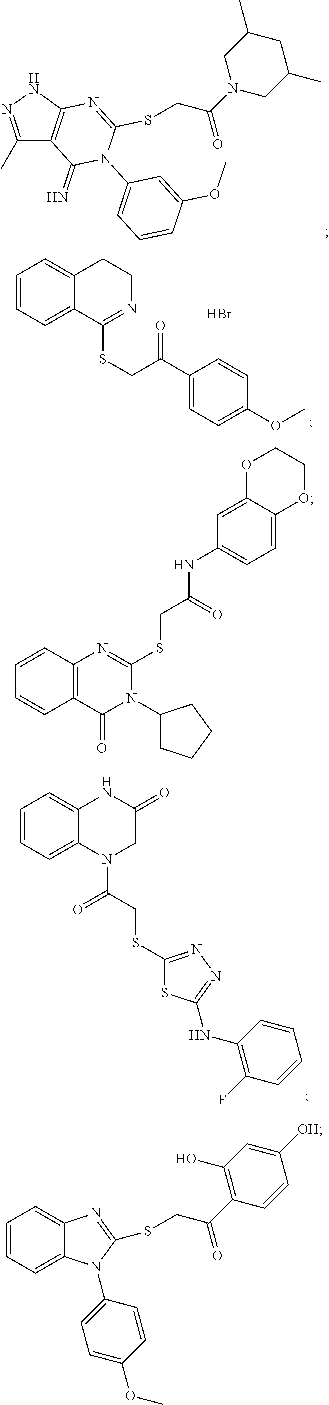

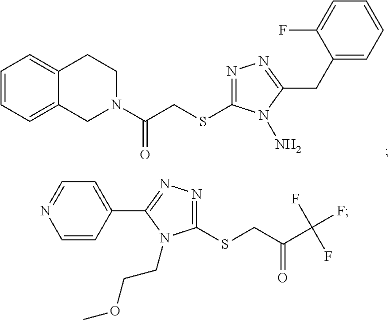

or a pharmaceutically acceptable salt or stereoisomer, including enantiomer, diastereomer, racemate or mixtures of stereoisomer, thereof. In one embodiment, the subject is a human.

In another embodiment, provided herein is a method of treating an influenza virus disease in a subject, comprising administering to a subject in need thereof an effective amount of a Compound, wherein the Compound has the structure:

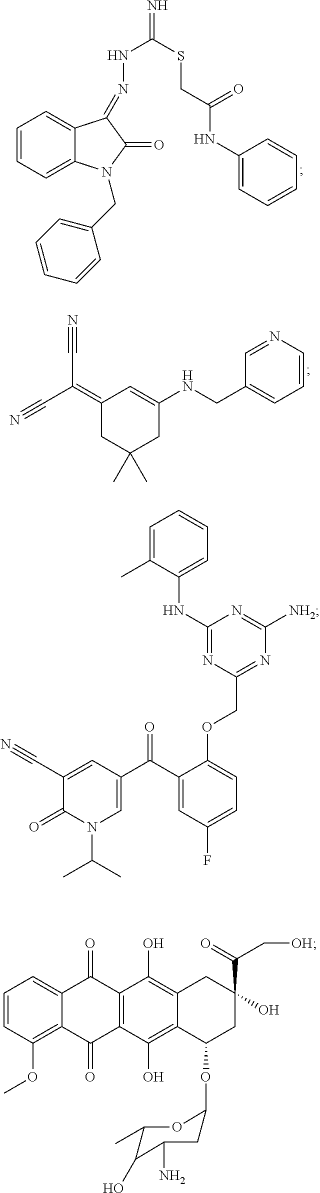

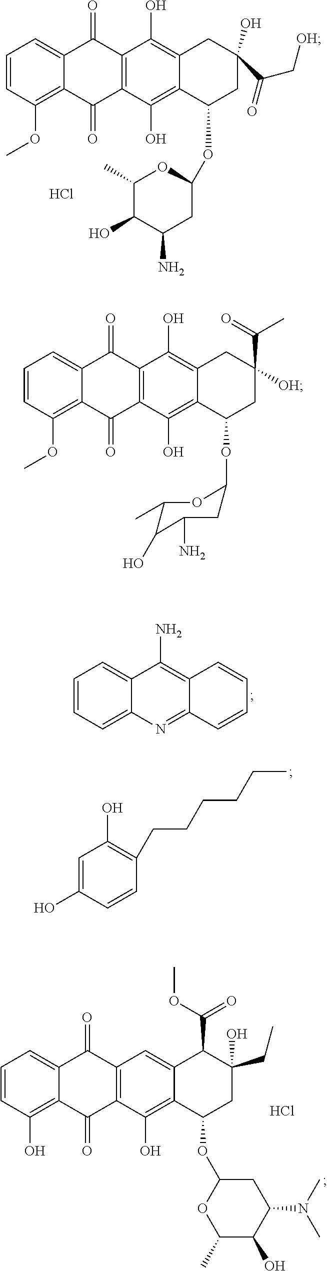

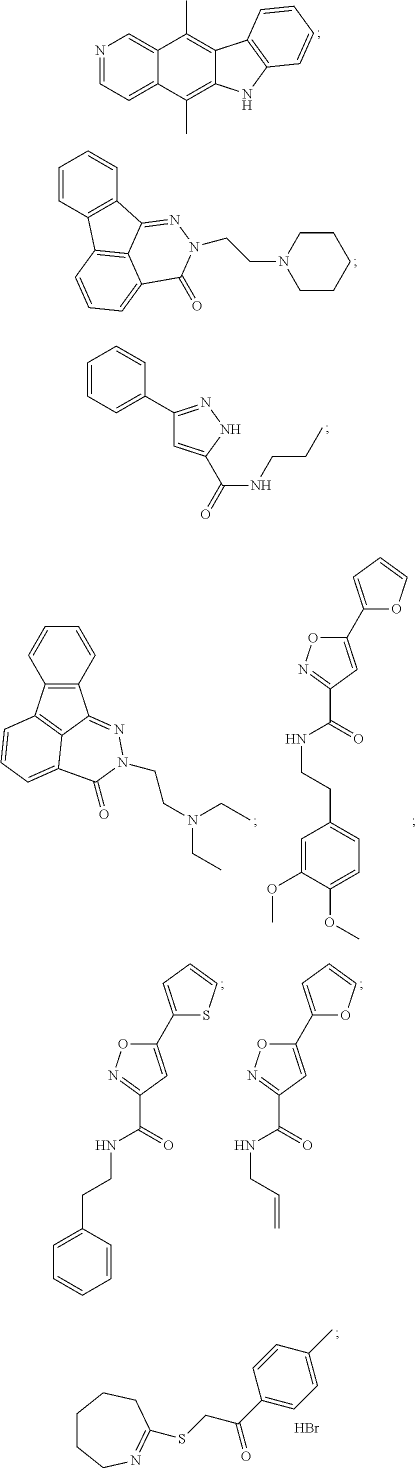

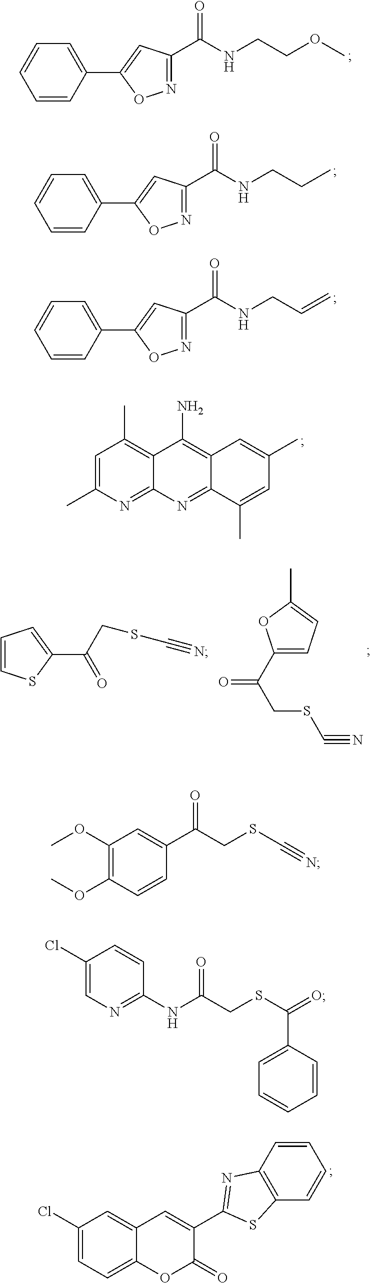

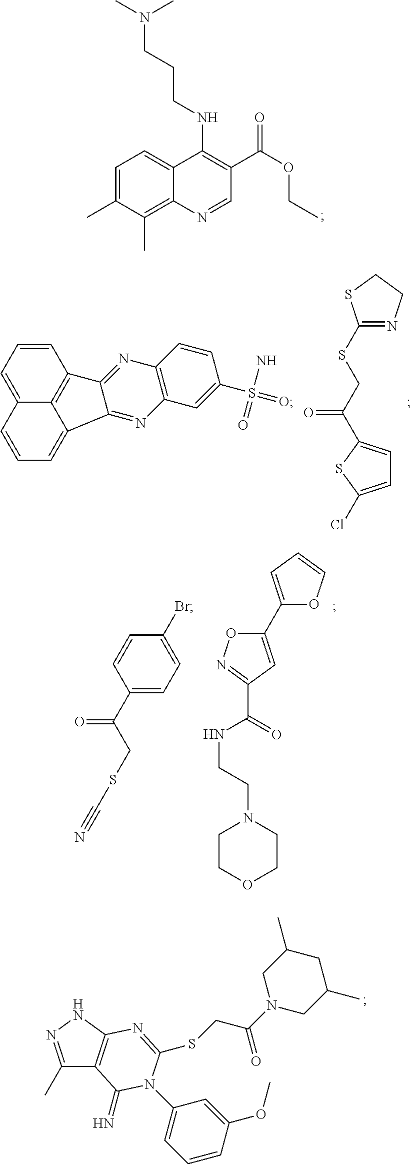

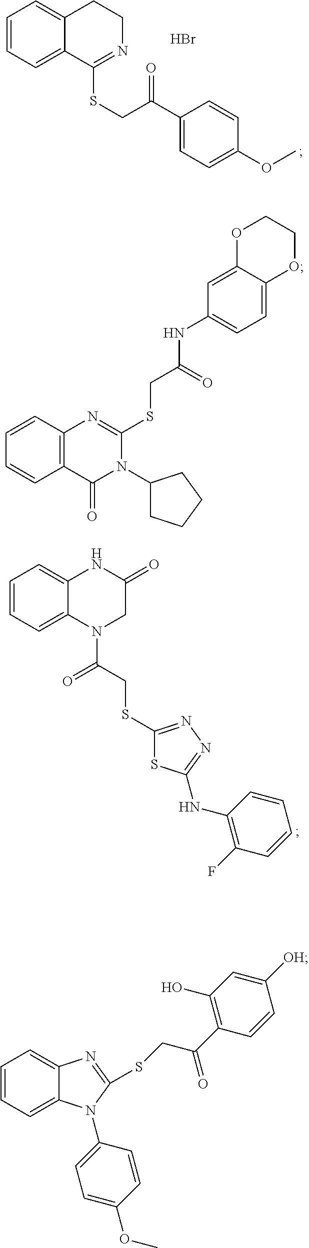

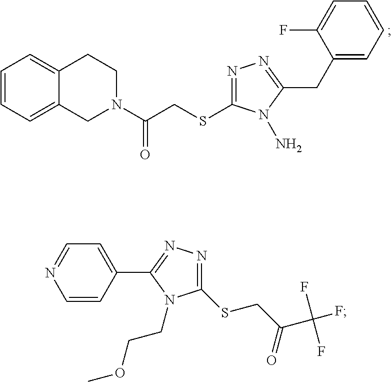

or a pharmaceutically acceptable salt or stereoisomer, including enantiomer, diastereomer, racemate or mixtures of stereoisomer, thereof. In one embodiment, the subject is a human.

In another embodiment, provided herein is a method of preventing a symptom associated with an influenza virus disease in a subject, comprising administering to a subject in need thereof an effective amount of a Compound, wherein the Compound has the structure:

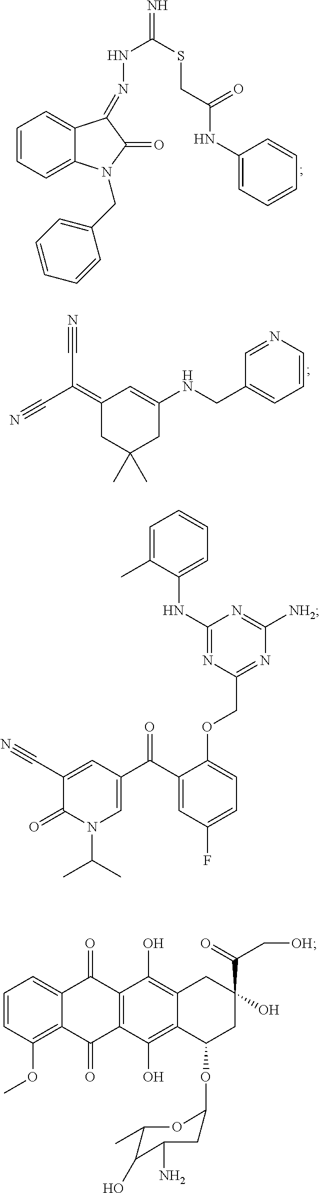

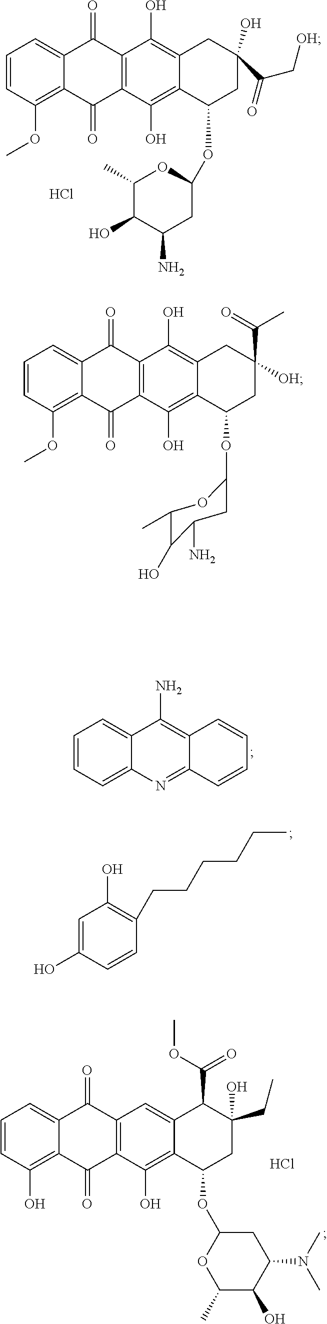

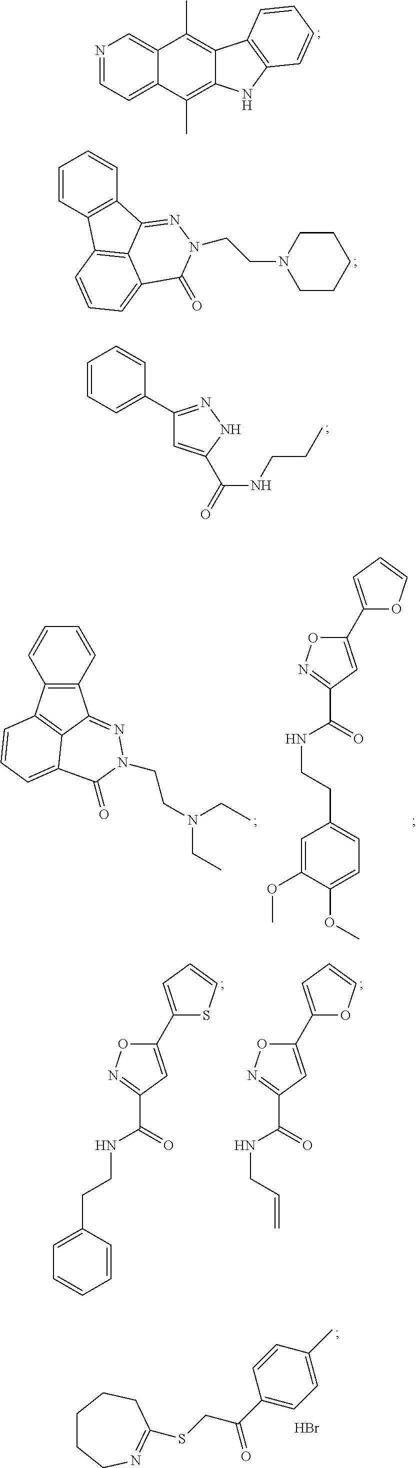

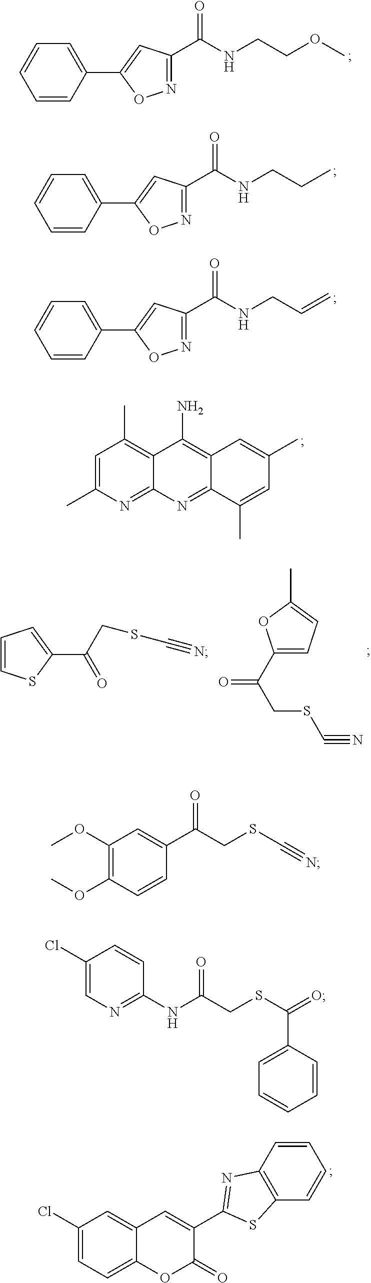

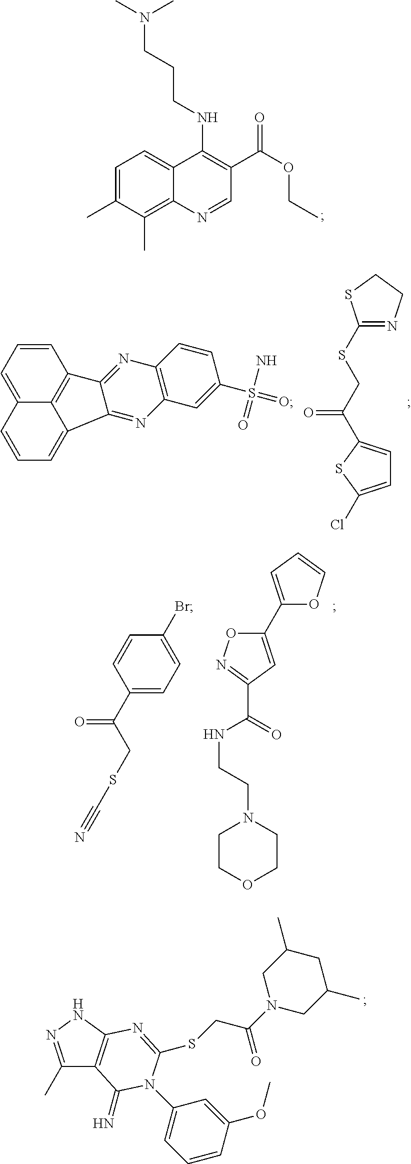

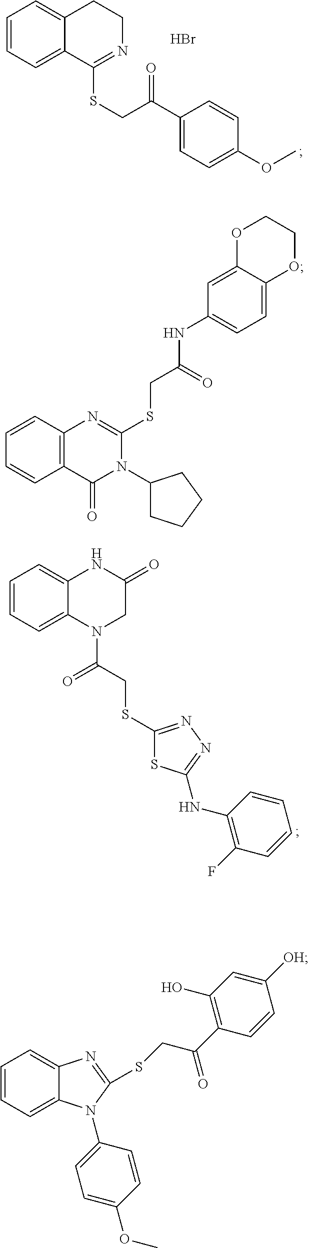

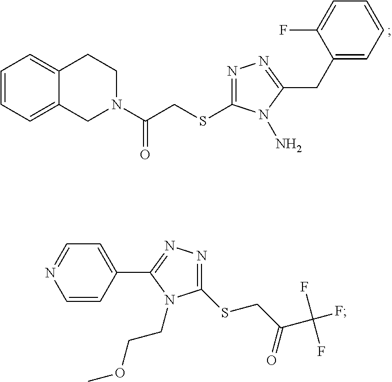

or a pharmaceutically acceptable salt or stereoisomer, including enantiomer, diastereomer, racemate or mixtures of stereoisomer, thereof. In one embodiment, the subject is a human.

In another embodiment, provided herein is a method for treating an interferon-sensitive disease, comprising administering to a subject in need thereof an effective amount of a Compound, wherein the Compound has the structure:

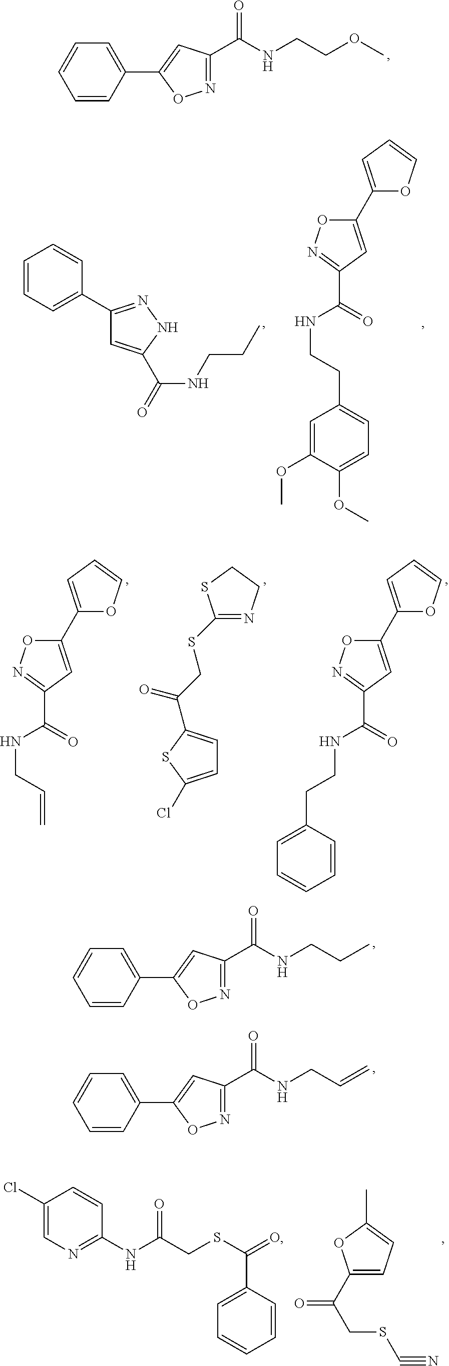

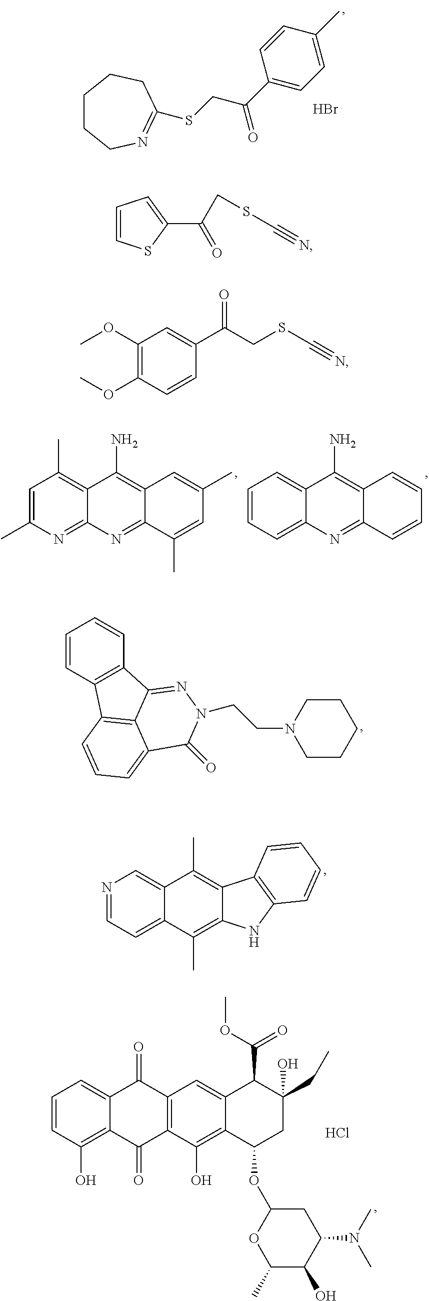

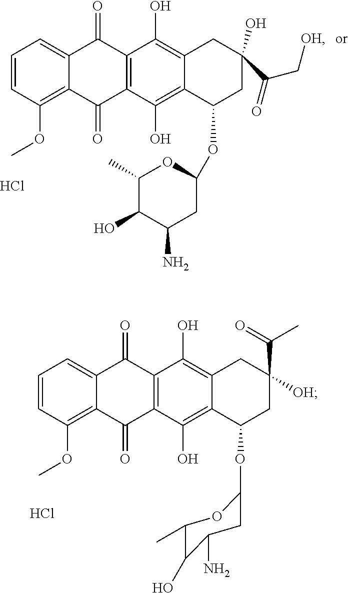

or a pharmaceutically acceptable salt or stereoisomer, including enantiomer, diastereomer, racemate or mixtures of stereoisomer, thereof. In one embodiment, the subject is a human.

In another embodiment, provided herein is a method for treating an interferon-sensitive disease, comprising administering to a subject in need thereof an effective amount of a Compound, wherein the Compound has the formula:

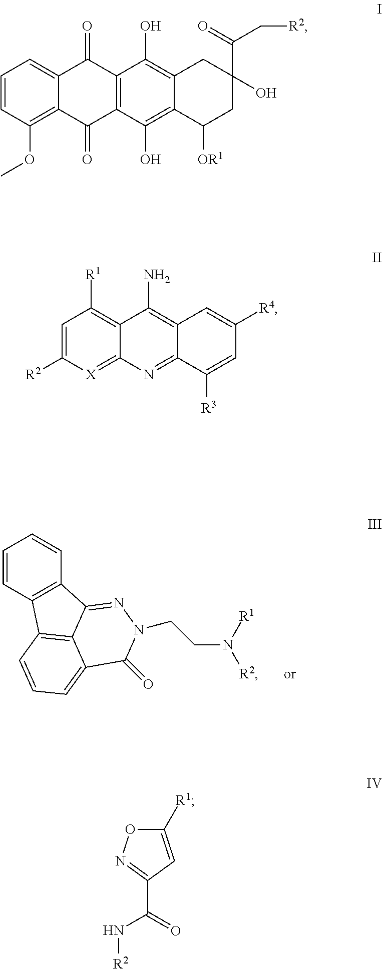

or a pharmaceutically acceptable salt or stereoisomer, including enantiomer, diastereomer, racemate or mixtures of stereoisomer, thereof; wherein

(i) R¹ of formula I is substituted or unsubstituted heterocyclyl; and R² of formula I is H or OH;

(ii) X of formula II is CH or N; and R¹, R², R³ and R⁴ of formula II are at each occurrence independently hydrogen or substituted or unsubstituted C₁₋₄ alkyl;

(iii) R¹ and R² of formula III are at each occurrence independently substituted or unsubstituted C₁₋₄ alkyl, or R¹ and R² of formula III taken together with the nitrogen atom to which they are attached form substituted or unsubstituted heterocyclyl; and

(iv) R¹ of formula IV is substituted or unsubstituted aryl or substituted or unsubstituted heteroaryl; and R² of formula IV is substituted or unsubstituted C₁₋₄ alkyl, substituted or unsubstituted C₂₋₄alkenyl or substituted or unsubstituted heteroaryl,

wherein the compound of formula I is not doxorubicin or daunorubicin and the compound of formula II is not aminacrine. In a particular embodiment,

(i) R¹ of formula I is substituted tetrahydro-2H-pyran;

(ii) R¹, R², R³ and R⁴ of formula II are methyl;

(iii) R¹ and R² of formula III are each ethyl, or R¹ and R² of formula III taken together with the nitrogen atom to which they are attached form piperidine; and

(iv) R¹ of formula IV is substituted or unsubstituted furan, thiophene or phenyl; and R² of formula IV is substituted or unsubstituted alkoxyalkyl, arylalkyl, allyl, propyl or alkoxyalkyl. In one embodiment, the subject is a human.

In another embodiment, provided herein is a method for treating an interferon-sensitive disease, comprising administering to a subject in need thereof an effective amount of a Compound, wherein the Compound has the structure:

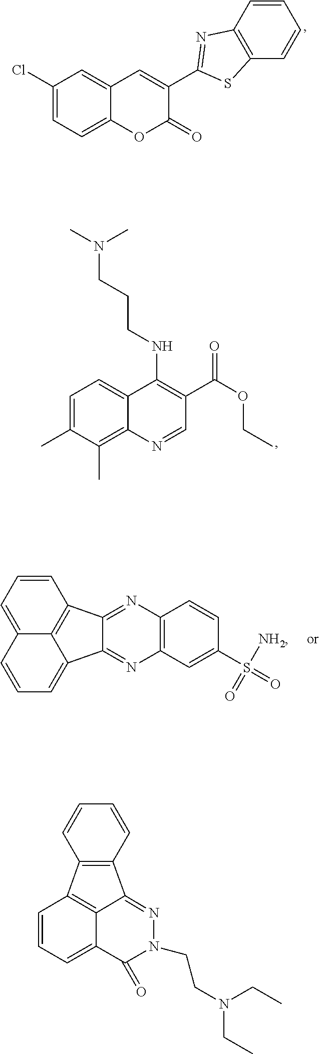

or a pharmaceutically acceptable salt or stereoisomer, including enantiomer, diastereomer, racemate or mixtures of stereoisomer, thereof. In one embodiment, the subject is human.

In another embodiment, provided herein is a method for treating an influenza virus disease, comprising administering to a subject in need thereof an effective amount of a Compound, wherein the Compound has the structure:

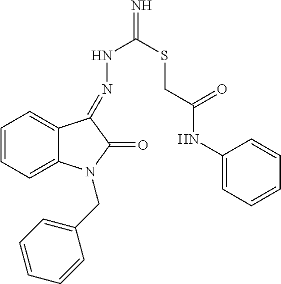

or a pharmaceutically acceptable salt or stereoisomer, including enantiomer, diastereomer, racemate or mixtures of stereoisomer, thereof. In a particular embodiment, the influenza virus is an influenza A virus. In one such embodiment, the influenza A virus is subtype H1N1, H3N2, or H5N1. In one embodiment, the subject is human.

In one of the foregoing embodiments, the interferon-sensitive disease is caused by a viral infection, such as, e.g., influenza virus, adenovirus, arbovirus, paramyxovirus, baculovirus, coronavirus, papillomavirus, parvovirus, chickenpox virus, reovirus, Ebola virus, Ebola-like virus, echo virus, encephalitis virus, filovirus, hantavirus, hepatitis virus, German measles virus, cytomegalovirus, hemorrhagic fever virus, herpes simplex virus, hepatitis B virus, hepatitis C virus, human immunodeficiency virus, human papillomavirus, human T cell leukemia virus, human T cell lymphoma virus, human T cell lymphotropic virus, Lassa fever virus, Marburg virus, measles virus, mumps virus, myxovirus, nairovirus, nanirnavirus, nariva virus, ndumo virus, Necrovirus, neethling virus, neopvirus, neurotropic virus, Newcastle disease virus, oncornavirus, orbivirus, orthomyxovirus, parainfluenza virus, paramyxovirus, parvovirus, picornavirus, rabies virus, respiratory syncytial virus, rhinovirus, rubella virus, rubeola virus, SARS virus, Sendai virus, simian immunodeficiency virus, simian parainfluenza virus, smallpox virus, varicella zoster virus, variola virus, or vesicular stomatitis virus.

In one of the foregoing embodiments, the interferon-sensitive disease is caused by a bacterial infection. In another embodiment, the interferon-sensitive disease is multiple sclerosis. In another embodiment, the interferon-sensitive disease is cancer.

In another embodiment, provided herein is a method of enhancing the immune response in a subject that has been administered a vaccine, comprising administering to the subject an effective amount of a Compound, wherein the Compound has the formula:

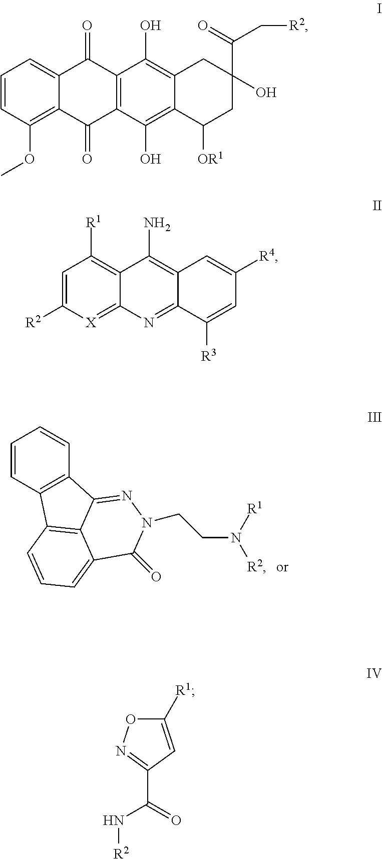

or a pharmaceutically acceptable salt or stereoisomer, including enantiomer, diastereomer, racemate or mixtures of stereoisomer, thereof; wherein

(i) R¹ of formula I is substituted or unsubstituted heterocyclyl; and R² of formula I is H or OH;

(ii) X of formula II is CH or N; and R¹, R², R³ and R⁴ of formula II are at each occurrence independently hydrogen or substituted or unsubstituted C₄ alkyl;

(iii) R¹ and R² of formula III are at each occurrence independently substituted or unsubstituted C₁₋₄ alkyl, or R¹ and R² of formula III taken together with the nitrogen atom to which they are attached form substituted or unsubstituted heterocyclyl; and

(iv) R¹ of formula IV is substituted or unsubstituted aryl or substituted or unsubstituted heteroaryl; and R² of formula IV is substituted or unsubstituted C₁₋₄ alkyl, substituted or unsubstituted C₂₋₄alkenyl or substituted or unsubstituted heteroaryl. In a particular embodiment,

(i) R¹ of formula I is substituted tetrahydro-2H-pyran;

(ii) R¹, R², R³ and R⁴ of formula II are methyl;

(iii) R¹ and R² of formula III are each ethyl, or R¹ and R² of formula III taken together with the nitrogen atom to which they are attached form piperidine; and

(iv) R¹ of formula IV is substituted or unsubstituted furan, thiophene or phenyl; and R² of formula IV is substituted or unsubstituted alkoxyalkyl, arylalkyl, allyl, propyl or alkoxyalkyl. In one embodiment, the subject is human.

In another embodiment, provided herein is a method for enhancing the immune response in a subject that has been administered a live virus vaccine, comprising administering to the subject an effective amount of a Compound, wherein the Compound has the structure:

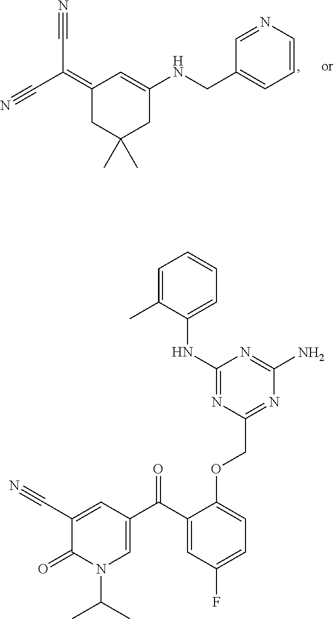

or a pharmaceutically acceptable salt or stereoisomer, including enantiomer, diastereomer, racemate or mixtures of stereoisomer, thereof. In one embodiment, the subject is human.

In another embodiment, provided herein is a method of enhancing the immune response in a subject that has been administered a live virus vaccine, comprising administering to the subject an effective amount of a Compound, wherein the Compound has the structure:

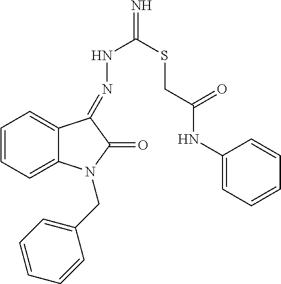

or a pharmaceutically acceptable salt or stereoisomer, including enantiomer, diastereomer, racemate or mixtures of stereoisomer, thereof. In one embodiment, the subject is human.

In another embodiment, provided herein is a method of enhancing the immune response in a subject that has been administered a vaccine, comprising administering to the subject an effective amount of a Compound, wherein the Compound has the structure:

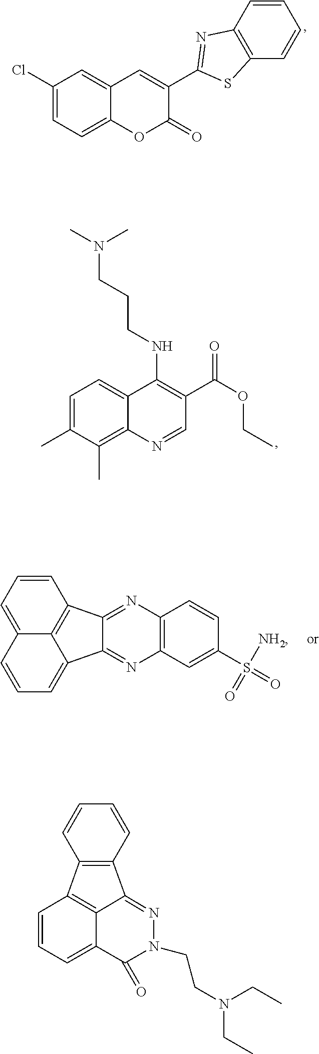

or a pharmaceutically acceptable salt or stereoisomer, including enantiomer, diastereomer, racemate or mixtures of stereoisomer, thereof. In one embodiment, the subject is human.

In another embodiment, provided herein is a method of enhancing the immune response in a subject that has been administered a vaccine, comprising administering to the subject an effective amount of a Compound, wherein the Compound has the structure:

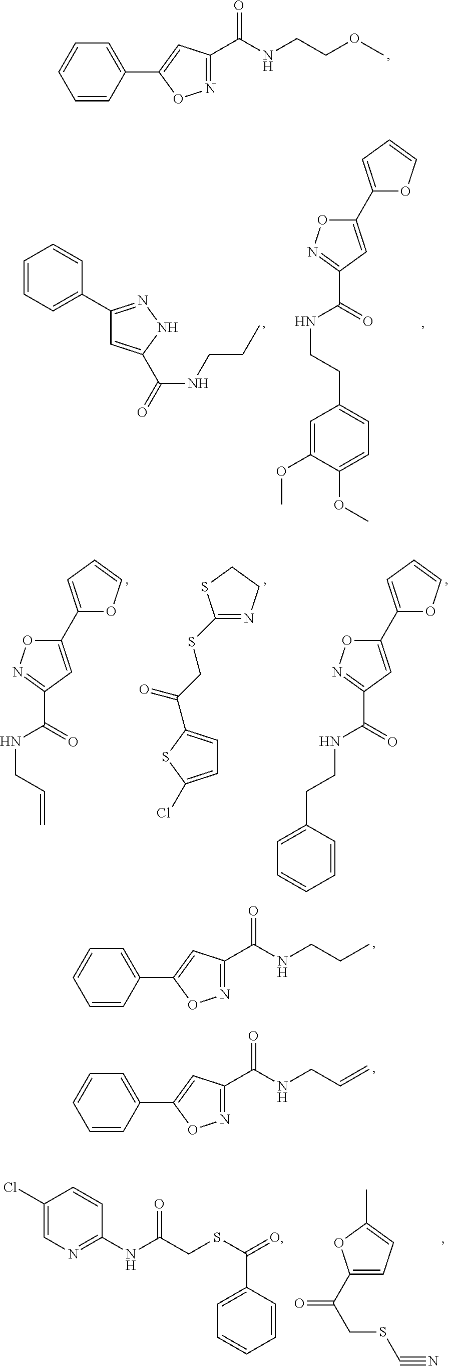

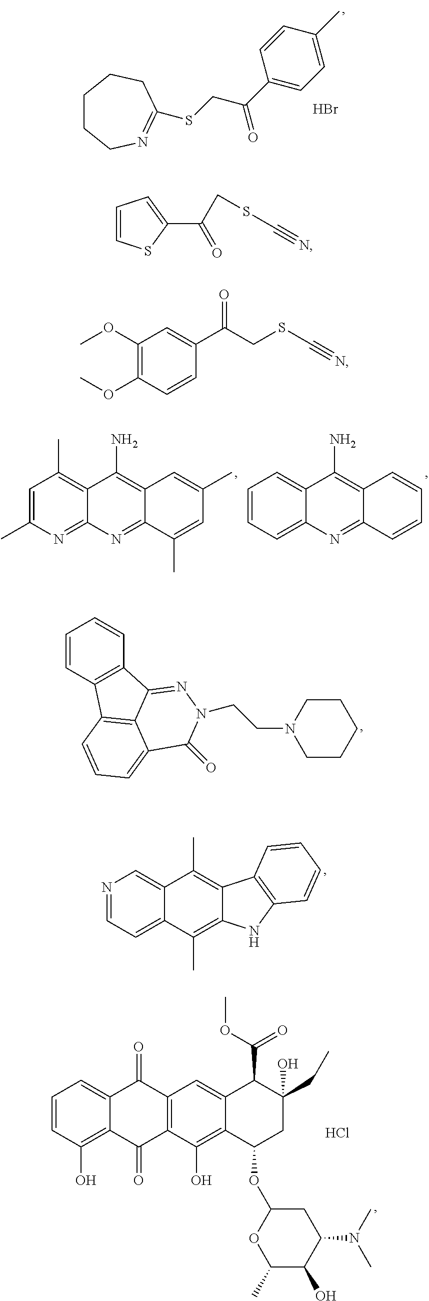

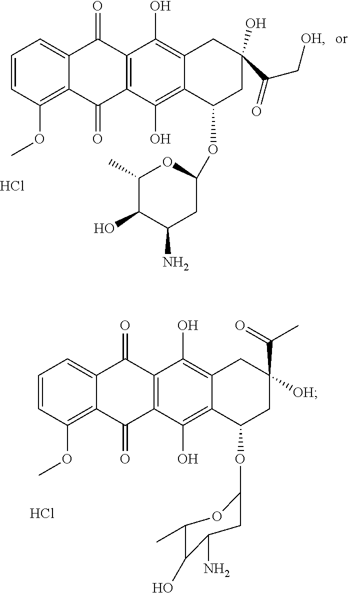

or a pharmaceutically acceptable salt or stereoisomer, including enantiomer, diastereomer, racemate or mixtures of stereoisomer, thereof. In one embodiment, the subject is human.

In one of the foregoing embodiments, the vaccine is an anthrax vaccine, BCG vaccine, Diphtheria vaccine, tetanus vaccine, pertussis vaccine, haemophilus B vaccine, hepatitis A vaccine, hepatitis B vaccine, human papillomavirus vaccine, influenza vaccine, Japanese encephalitis virus vaccine, measles vaccine, mumps vaccine, plague vaccine, pneumococcal vaccine, poliovirus vaccine, rabies vaccine, respiratory syncytial virus vaccine, rotavirus vaccine, rubella vaccine, smallpox vaccine, typhoid vaccine, varicella virus vaccine, yellow fever vaccine, or zoster vaccine. In a particular embodiment, the vaccine is an influenza vaccine.

In another embodiment, provided herein is a method for treating or preventing a viral disease in a subject in need thereof, comprising administering to the subject an effective amount of a Compound, wherein the Compound has the structure:

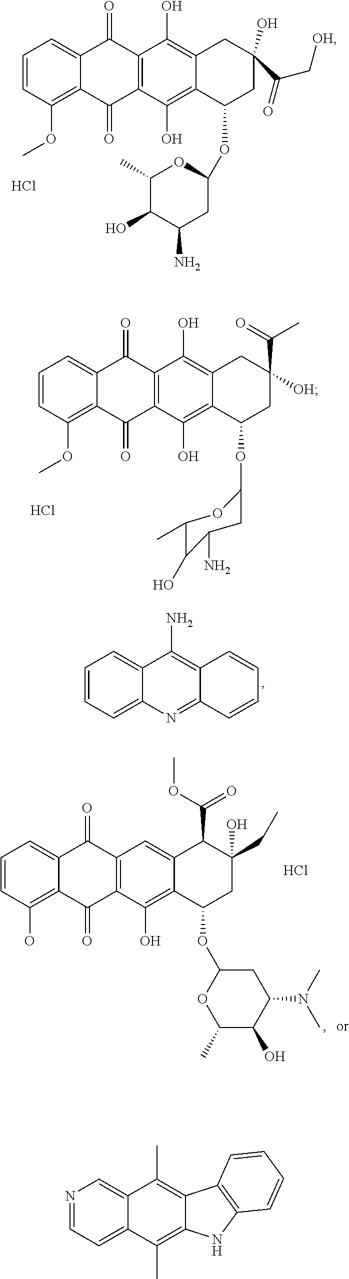

or a pharmaceutically acceptable salt or stereoisomer, including enantliomer, diastereomer, racemate or mixtures of stereoisomer, thereof. In one embodiment, the subject is human. In one embodiment, the subject is cancer-free.

In a particular embodiment, the viral disease is caused by influenza virus, adenovirus, arbovirus, paramyxovirus, baculovirus, coronavirus, papillomavirus, parvovirus, chickenpox virus, reovirus, Ebola virus, Ebola-like virus, echo virus, encephalitis virus, filovirus, hantavirus, hepatitis virus, German measles virus, cytomegalovirus, hemorrhagic fever virus, herpes simplex virus, human immunodeficiency virus, human papillomavirus, human T cell leukemia virus, human T cell lymphoma virus, human T cell lymphotropic virus, Lassa fever virus, Marburg virus, measles virus, mumps virus, myxovirus, nairovirus, nanirnavirus, nariva virus, ndumo virus, Necrovirus, neethling virus, neopvirus, neurotropic virus, Newcastle disease virus, oncornavirus, orbivirus, orthomyxovirus, parainfluenza virus, paramyxovirus, parvovirus, picornavirus, rabies virus, respiratory syncytial virus, rhinovirus, rubella virus, rubeola virus, SARS virus, Sendai virus, simian immunodeficiency virus, simian parainfluenza virus, smallpox virus, varicella zoster virus, variola virus, or vesicular stomatitis virus.

### 6. EXAMPLES

The following examples are offered by way of illustration, and not by way of limitation. The examples describe the identification of compounds that induce interferon production and the effectiveness of certain of such compounds to inhibit viral replication.

6.1 Identification of Compounds that Induce Interferon Production and Inhibit Viral Replication

6.1.1 μMaterials and Methods

6.1.1.1 μMammalian Cell Lines and Viruses

A Madin-Darby canine kidney (MDCK) epithelial cell line that stably expresses the firefly luciferase enzyme under the strict regulation of the IFN beta promoter (MDCK-IFNb-Luc) was generated as previously described (see Hai et al., J Virol, 2008. 82(21):10580-90). The MDCK-NS1 cell line stably expresses the A/PR/8/34 NS1 protein fused to GFP. MDCK cells, human embryonic kidney 293T cells, human alveolar basal epithelial (A549) cells, and African green monkey kidney (Vero) cells were obtained from the American Type Culture Collection (ATCC, Manassas, Va.) and were maintained in Dulbecco's modified Eagle's medium (DMEM) or minimal essential medium (both from GIBCO, Carlsbad, Calif.), each supplemented with 10% fetal bovine serum (FBS) (HyClone, Logan, Utah) and 1% penicillin-streptomycin (GIBCO).

Influenza A/PR/8/34 virus was propagated in 10-day-old embryonated chicken eggs for 2 days at 37° C. Recombinant A/PR/8/34 NS1 mutant virus deltaNS1 (containing a complete deletion of the NS1 expressing gene) was propagated in 6-day-old embryonated chicken eggs for 2 days at 37° C. Other A/PR/8/34 NS1 mutant viruses NS1-73 (expressing NS1 amino acids 1-73), NS1-113 (expressing NS1 amino acids 1-113), NS1-126 (expressing NS1 amino acids 1-126) were propagated in 8-day-old embryonated chicken eggs for 2 days at 37° C. Influenza A/WSN/33 virus was grown in MDCK cells in DMEM post-infection medium (DMEM supplemented with 0.3% bovine serum albumin (BSA), 0.1% FBS, 10 mM HEPES, 1% penicillin-streptomycin, and 1 ug/mL TPCK). A/WSN/33 with a deleted NS1 protein was grown in MDCK-NS1 cells in DMEM post-infection medium. All wild-type influenza viruses were titered by standard plaque assay in MDCK cells. NS1 mutant viruses were titered by plaque assay in the MDCK-NS1 cells. Vesicular stomatitis virus expressing the green fluorescence protein (VSV-GFP) was kindly provided by John Hiscott (McGill University, Montreal, Canada) and was grown and titered by plaque assay in Vero cells. NDV-GFP was propagated in 8-day old embryonated chicken eggs (Charles River Laboratories; North Franklin, Conn.).

6.1.1.2 Small Molecular Weight Compounds

All Compounds were provided by and screened at the National Screening Laboratory for the Regional Centers of Excellence in Biodefense and Emerging Infectious Diseases (NSRB), located at Harvard Medical School, Boston, Mass. The libraries screened are the following: Biomol ICCB Known Bioactives2 Library (480 Compounds; BIOMOL, Plymouth Meeting, Pa.), NINDS custom collection 2 (1,040 Compounds; MicroSource Discovery Systems, Gaylorsville, Conn.), Prestwickl Collection (1,120 Compounds; Prestwick Chemical, Inc., Washington, D.C.), ActiMol Tim Tec 1 (8,518 Compounds; TimTec Inc., Newark, Del.), Asinex 1 (12,378 Compounds; Asinex Corp., Winston-Salem, N.C.), Bionet 2 (1,700 Compounds; Ryan Scientific, Mount Pleasant, S.C.), Chembridge 3 (10,560 Compounds; ChemBridge Corp., San Diego, Calif.), ChemDiv 2 (8,560 Compounds; ChemDiv, Inc., San Diego, Calif.), ChemDiv 4 (1,320 Compounds; ChemDiv, Inc., San Diego, Calif.), Enamine 2 (26,576 Compounds; ENAMINE Ltd., Kiev, Ukraine), Life Chemicals 1 (3,893 Compounds; Life Chemicals Inc., Burlington, ON), Maybridge 4 (4,576 Compounds; Maybridge Ltd., Trevillett, England), Maybridge 5 (3,212 Compounds; Maybridge Ltd., Trevillett, England), Mixed Commercial Plate 5 (268 Compounds from ChemDiv and Maybridge libraries), Peakdale 2 (352 Compounds; Peakdale Molecular Ltd. Chapel-en-1e-Frith, High Peak, UK). The Compounds were dissolved in DMSO at library-defined concentrations.

For secondary analyses, hit Compounds were purchased directly from the vendors indicated and dissolved in DMSO. The final concentration of DMSO in the culture medium did not exceed 0.5%.

6.1.1.3 High-Throughput Screen (HTS)

The primary screen was performed in 384-well format, using solid white 384-well tissue culture-treated plates (Corning Life Sciences, Lowell, Mass.). MDCK-IFNb-Luc cells were cultured to 95% confluency, trypsinized with 0.05% Trypsin-EDTA (Invitrogen Corp., Carlsbad, Calif.), and resuspended in phenol red-free DMEM growth medium supplemented with 5% FBS, and 1% penicillin-streptomycin at 2×10⁵ cells/mL. These were then transferred into 384-well plates using the Matrix Wellmate (Thermo Fisher Scientific Inc., Hudson, N.H.) plate filler. Loaded plates were centrifuged at 1000 rpm for 1 min to ensure that all cells and media were at the bottom of each well. The cells were incubated for 20-24 hours at 37° C., 5% CO₂ before the addition of 100 nL of Compounds directly into each well by the Epson Compound transfer robot (Epson America, Inc., Long Beach, Calif.). The cells were incubated for 2 hours before infection with influenza A/PR/8/34 virus directly into the medium at an MOI of 10 using the Matrix Wellmate plate filler to transfer 5 μL of virus inoculum. This virus was diluted from its stock concentration with phenol red-free DMEM growth medium supplemented with 1% penicillin-streptomycin, and 7 μg/mL TPCK (to achieve a final concentration of 1 μg/mL TPCK in each well containing a total volume of 35 μL). Infected plates were subsequently centrifuged at 1000 rpm for 1 min. Control wells were included in each plate and these did not contain Compounds. The negative control wells were infected with A/PR/8/34 virus and positive control wells were infected with A/PR/8/34 NS1-113 mutant virus. Infection was allowed to proceed for 18-20 hours at 37° C., 5% CO₂. The plates were then removed from the incubator to equilibrate to room temperature for 20 min. The Matrix Wellmate plate filler was used to add 50 μL of BrightGlo Luciferase reagent (Promega Corp., Madison, Wis.) that was previously diluted with PBS to 3 times its volume. Upon incubation at room temperature for about 10 min, luminescence was measured for 0.1 s/well with the Envision plate reader (PerkinElmer Inc., Waltham, Mass.). This primary screen was performed in duplicate, where each set of Compounds was simultaneously arrayed in two separate plates, and each was run independently.

6.1.1.4 HTS analysis and Hit Definition

Data from the HTS assay were analyzed with Microsoft Office Excel. To evaluate the robustness of the assay, the statistical parameter Z′ factor (see Zhang et al., J Biomol Screen, 1999. 4(2):67-73) was calculated for each plate, based on the following formula: Z′=1−(3σₚₒₛ+3σ_{neg})/.(μₚₒₛ−μ_{neg}), where σₚₒₛ and σ_{neg} are the standard deviations for the positive and negative control wells, respectively, and μₚₒₛ and μ_{neg} are the average for the positive control and negative controls, respectively. An assay was considered an optimal assay if its Z′ factor at least 0.5.

To normalize the raw values obtained from the HTS, the statistical parameter Z-score was calculated for each individual Compound in a particular plate, based on the following formula: z=(x−μΥ)/δ, where x is the relative luciferase unit obtained from the plate reader, μ is the mean of all Compound-containing well of a particular plate, and δ is the standard deviation of all Compound-containing well of a particular plate.

Compound hits from the HTS were defined based on their Z-score. The criteria used included Compound that had a Z score of 3 and above, with its duplicate being at least 2.5.

6.1.1.5 Cell Viability Assay

The CellTiter-Glo® Luminescent Cell Viability Assay (Promega Corp., Madison, Wis.) was used to detect cell viability in accordance with the manufacturer's specifications. Briefly, MDCK-IFNb-Luc cells were plated in solid white 96-well plates (Corning Life Sciences, Lowell, Mass.) at 7.5×10³ cells per well, and incubated for 20-24 hours at 37° C., 5% CO₂. The medium was removed gently using a multichannel pipet and replaced with 50 μL of Compounds diluted at various concentrations, in phenol red-free DMEM, supplemented with 5% FBS, 1% penicillin-streptomycin. Upon a further 24 hour incubation, 50 μL of the CellTiter-Glo®solution (previously diluted in PBS to 2 times its volume) was added to each well. Upon incubation at room temperature for about 10 min, luminescence was measured for 0.1 s/well with the Beckman Coulter DTX 880 plate reader (Beckman Coulter, Inc., Fullerton, Calif.). The 10% cell cytotoxicity (CC₁₀) value was calculated. All values were normalized to the average value obtained from untreated cells.

6.1.1.6 Confirmation Screen

Verification of Compound hits obtained from the primary HTS was performed applying the same HTS protocol. The differences between the confirmation screen and the primary HTS are the following: 1) 96-well format was used, instead of 384-well format, 2) different Compound concentrations were tested, using each Compound's CC₁₀ as the median concentration, 3) compound concentrations were tested for their ability to induce IFN in the presence and absence of A/PR/8/34 infection, 4) each set was done in triplicate.

6.1.1.7 Interferon MDCK Bioassay

Verification of the ability of the selected Compounds obtained in the confirmation screen to induce IFN production was performed in a functional interferon bioassay as previously described (see Park et al., J Virol, 2003. 77(2):1501-11; and Iwata et al., J Vet Med Sci, 1996. 58(1):23-7), with some variations. Briefly, 96-well plates were seeded with MDCK cells at 1×10⁴ cells per well and allowed to incubate for 20-24 hours at 37° C., 5% CO₂. These were then treated with Compounds at 5 different dilutions, starting from the dilution of maximum reporter signal obtained from the confirmation screen. Two hours after treatment, these cells were either mock infected or infected with A/PR/8/34 virus directly into the media at an MOI of 10 and incubated for 18-20 hours at 37-C, 5% CO₂. A positive control included untreated cells infected with the A/PR/8/34 NS1-113 virus. Supernatant from these treated/infected MDCK cells (containing interferon) were transferred into a new 96-well plate and UV inactivated. This was done by placing the entire plate in a UV chamber, and delivering 200 J/cm² of energy. Two-fold dilutions were made with the UV-inactivated supernatants in DMEM containing 5% FBS. These dilutions were overlaid onto fresh MDCK cells that were plated the previous day, and left to incubate for 20-24 hours at 37° C., 5% CO₂. These were then infected with a GFP expressing vesicular stomatitis virus (VSV-GFP), which is sensitive to the effects of IFN and will only replicate if the supernatant from the MDCK influenza infected cells did not contain IFN protein. VSV-GFP replication was monitored by measuring the relative fluorescence of the GFP in a plate reader.

6.1.1.8 Viral growth assays in the presence of Compounds

MDCK cells were seeded onto 6-well plates and incubated for 24 hours at 37-C, 5% CO₂. The cells were washed with PBS and the medium was replaced with DMEM post-infection media containing various concentrations of the Compound of interest. These were incubated for 2 hours prior to infection with A/PR/8/34 or A/WSN/33 viruses at an MOI of 0.01 or 0.001, respectively. NS1 deleted mutant viruses (A/PR/8/34 delNS1 and A/WSN/33 delNS1) were used as negative controls for each wild type counterpart. Various timepoints were taken during virus growth and monitored with standard hemagglutination (HA) assay.

6.1.2 Results

6.1.2.1 High-Throughput Screen of Compounds that Restore IFN Production to Influenza Virus Infection

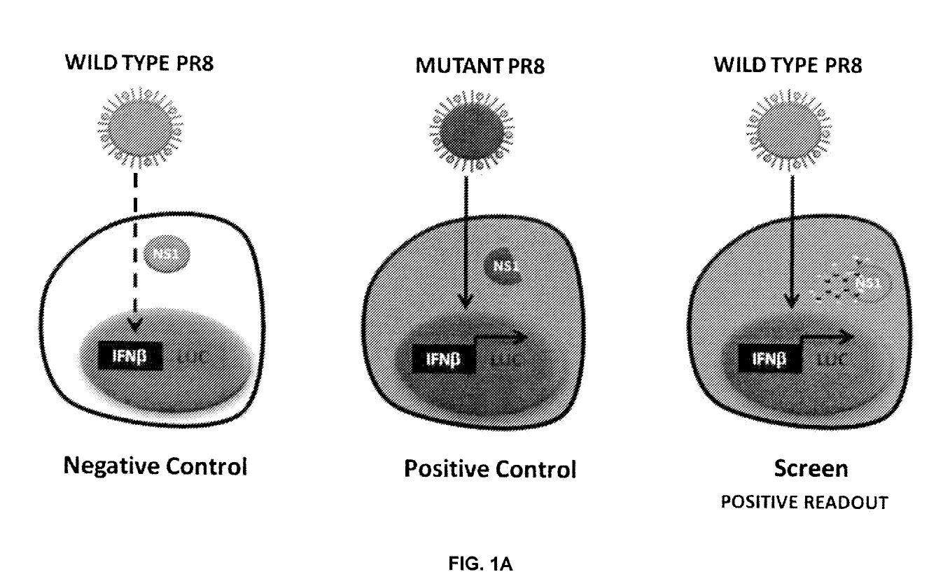

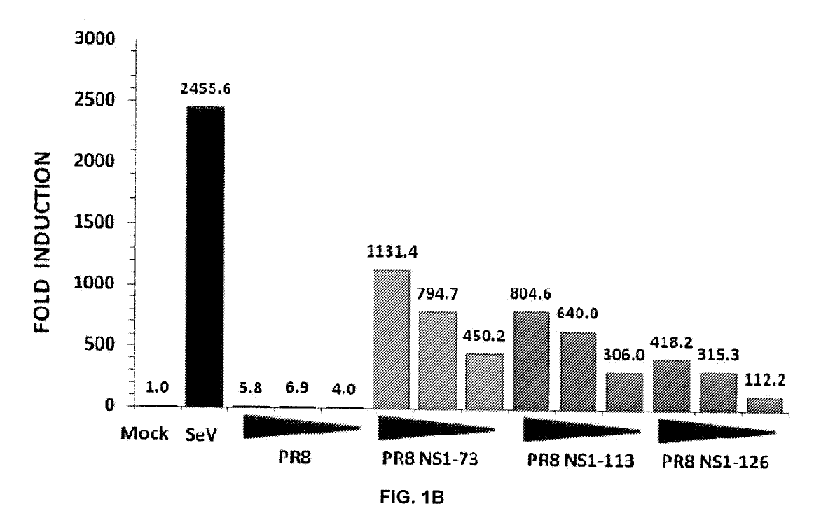

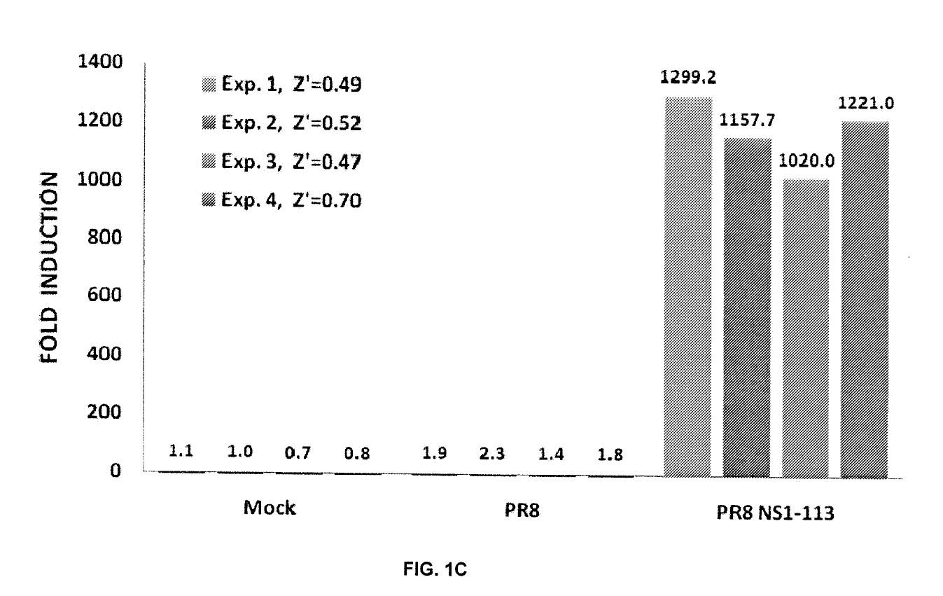

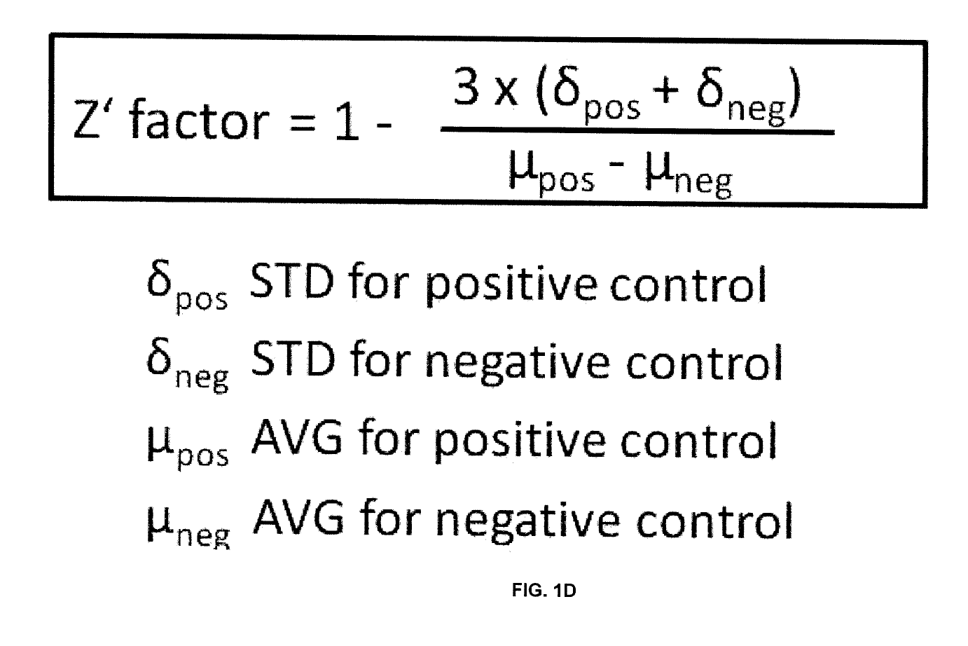

A cell-based HTS assay was developed for the identification of small molecular weight Compounds that induce IFN-I in the presence of influenza virus infection. An MDCK cell line that stably expresses the firefly luciferase enzyme under the strict regulation of the IFN beta promoter (MDCK-IFNb-Luc) was generated. Luciferase activity provides a measurement of the IFN beta response to influenza viruses expressing a non-functional NS1 protein. In the absence of the NS1 protein, the cytoplasmic sensor retinoic acid-inducible gene I (RIG-I) is able to detect the 5′ triphosphate moiety present in the RNA genome of influenza segments, and trigger downstream signaling, activating transcription factors, IRF3, NFkB, and AP1, required to turn on the IFNb promoter. Infection with wild-type influenza viruses does not induce the reporter due to the presence of the virally encoded NS1 protein, which blocks the IFNb induction pathway at several stages (see Hale et al., J Gen Virol, 2008. 89(Pt 10):2359-76). A positive readout in this screen will occur only in the presence of Compounds that target the NS1 molecule directly, host factors that are required for NS1 function, or modulators of the IFN beta production pathway independent of NS1 (FIG. 1). This screen will also capture false positive hits, involving Compounds that induce general cellular transcription or stabilize the luciferase enzyme. These will be discarded in secondary assays.

In order to confirm the validity of the cell line for HTS, infection with Sendai Virus Cantell (SeV-C), which is a known IFN beta inducer, yielded very high amounts of luciferase production (FIG. 1B). In contrast, when this cell line was infected with A/PR/8/34 virus at different multiplicities of infection, luciferase production was barely seen. In order to confirm that this inhibition of luciferase production is due to the antagonist function of the NS1 protein, the cell line was infected with A/PR/8/34 virus containing different truncations of the NS1 protein: 73, 113, and 126 amino acids instead of the full length 230 amino acids. Luciferase production was rescued upon infection of these truncated viruses, therefore confirming the antagonism of IFN beta by NS1. Most importantly, these results correspond to published literature, describing the attenuated phenotype of A/PR/8/34 NS1 truncation mutants, and the resulting induction of more IFN, as compared to wild type virus.

This assay was initially optimized in 96-well format and its validity was confirmed by demonstrating a Z′ factor (see Zhang et al., J Biomol Screen, 1999. 4(2):67-73) of at least 0.5. For HTS, the assay was further miniaturized and validated in 384-well format during a pilot screen conducted at the National Screening Laboratory for the Regional Centers of Excellence in Biodefense and Emerging Infectious Diseases (NSRB), housed at Harvard Medical School, using available robotic equipment for cell plating and Compound transfer.

6.1.2.2 Identification of IFN-Inducing Compounds

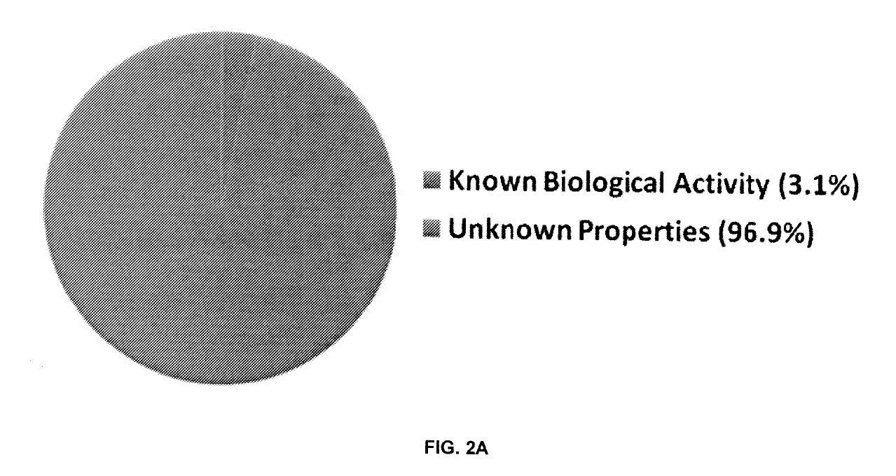

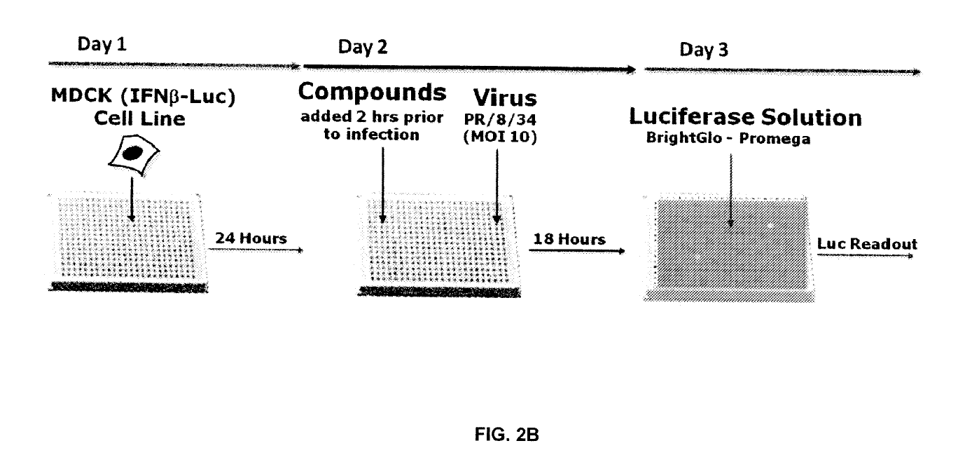

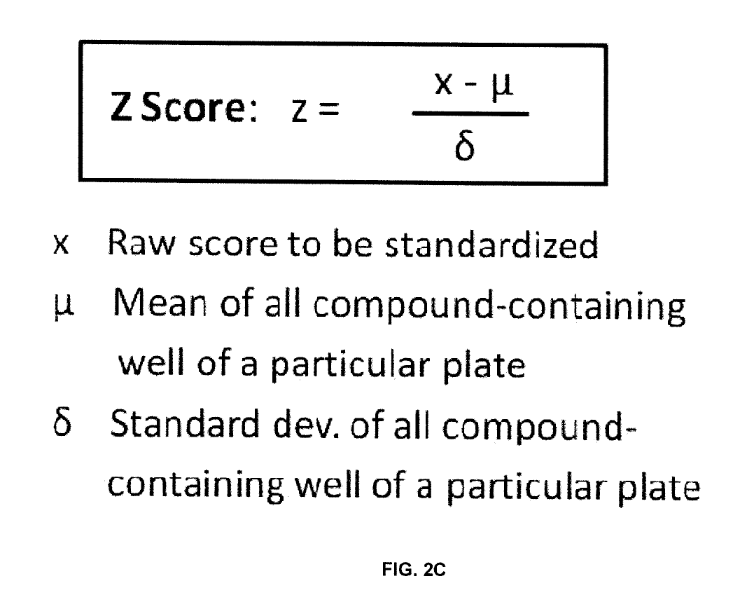

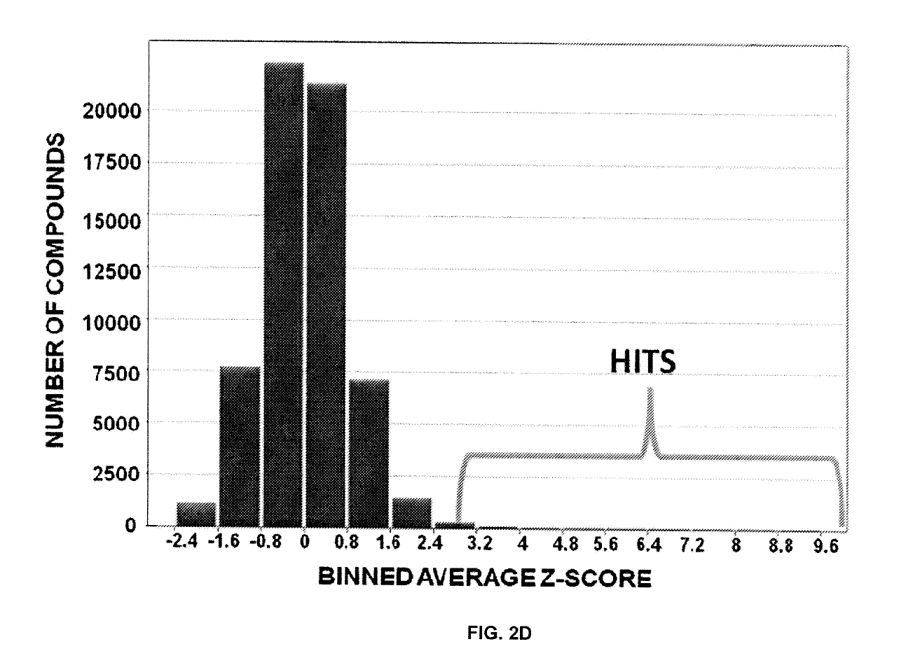

A total of 84,553 Compounds were screened from 15 different Compound libraries, 3 of which fall under the classification of Compounds with known biological activity. This class contains Compounds whose targets are known and 47% of them are FDA-approved drugs. Screening these types of Compounds may facilitate downstream analyses if chosen as hits. The remaining 12 libraries fell into the classification of Compounds with unknown properties (FIG. 2A).

Analysis of the results obtained from the Compound screen was based on the Z score for each Compound. The Z score indicates how many standard deviations a particular Compound is above or below the mean of the plate (FIG. 2C). For this analysis, the mean and standard deviation of all Compound-containing wells of each plate were first calculated. Then, the Z score for each Compound-containing well was calculated using the mean and standard deviation of its respective plate. Duplicate plates were treated separately, and their Z scores were compared for reproducibility of the effect. Since the assay is based on gain-of-function, only positive Z scores were focused on.

The definition of a “hit” was very stringent. A Z score of 3 was calculated to be equivalent to a statistically significant P value of 0.0214. Therefore, a Compound to be a hit was defined as one that contained a Z score of 3 and above, with its duplicate being at least 2.5. Applying these criteria to all the tested Compounds, 261 hits were identified from the 84,551 Compounds screened (FIG. 2D). This is approximately a 0.3% hit rate which is considered optimal in small molecule high-throughput screens.

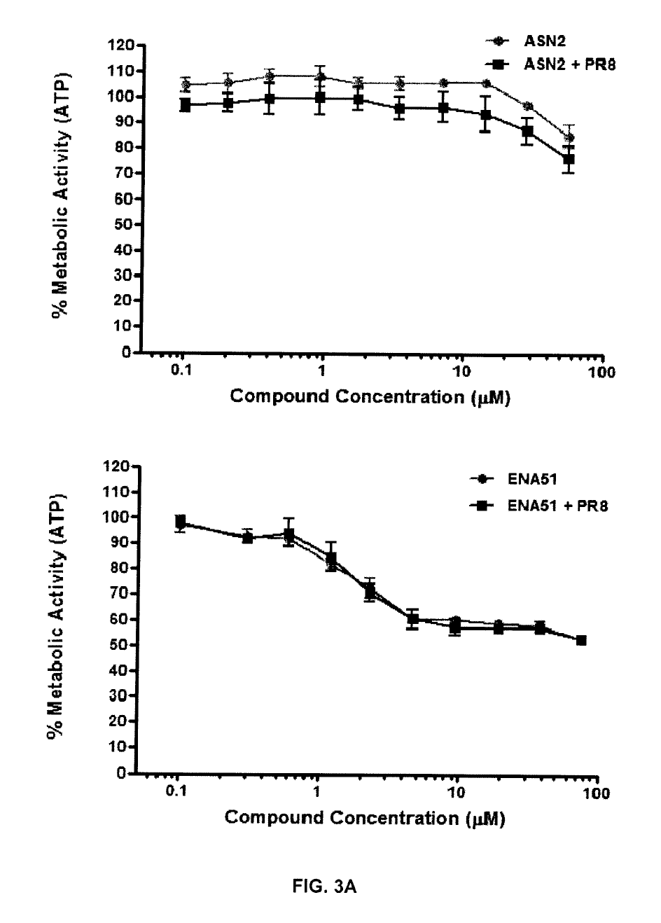

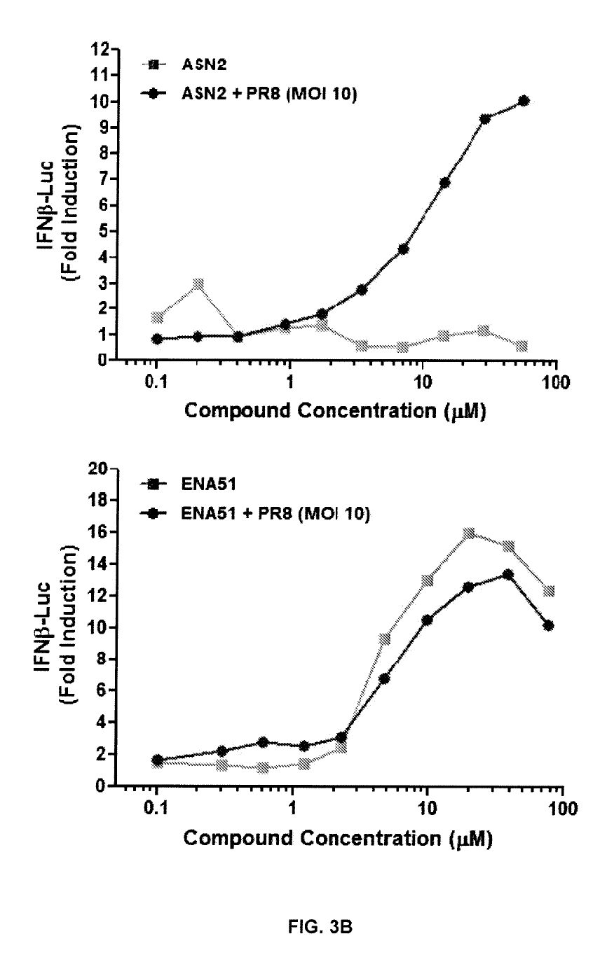

From the 261 Compound hits identified in the primary screen, 250 were available for purchase commercially as purified powders. To rule out false positive hits, a confirmation screen was run manually in 96-well format for all 250 purchased Compounds. First, the 10% cell cytotoxicity value (CC₁₀) was determined for each Compound by measuring the relative ATP levels of 24 hour-treated MDCK-IFNb-Luc cells. The confirmation screen, in contrast to the primary screen, tested 10 different Compound concentrations, 5 two-fold dilutions above and 5 two-fold dilutions below each Compound's CC₁₀. In addition to the Compound concentration range, the confirmation screen also tested each Compound for induction of IFNb in the absence or presence of PR/8/34 infection, in order to identify Compounds that induce IFNb independent of a virus stimulus. From this confirmation screen, 27 Compounds that significantly induce the IFNb-Luc reporter were identified. Three Compounds induced IFNb in a virus-dependent manner, and the remaining 24 induced IFN independently of influenza virus infection (FIG. 3).

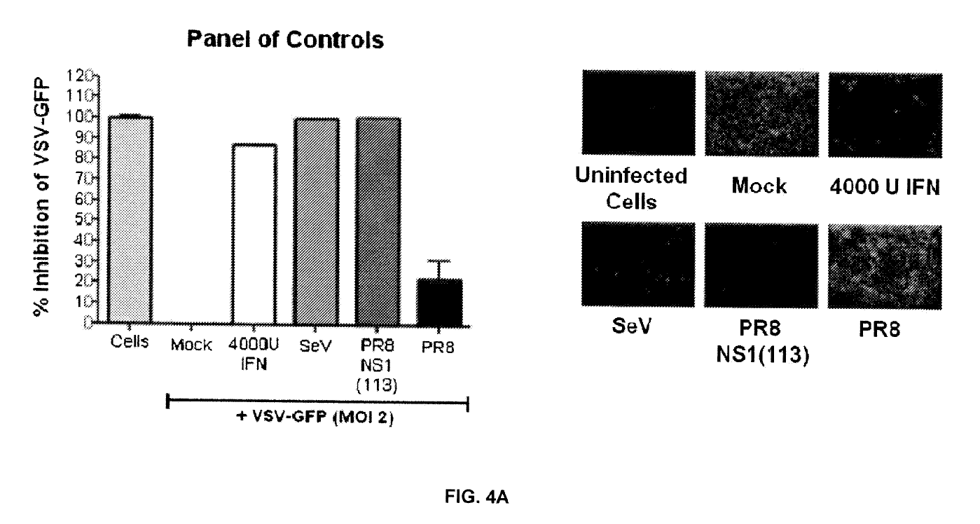

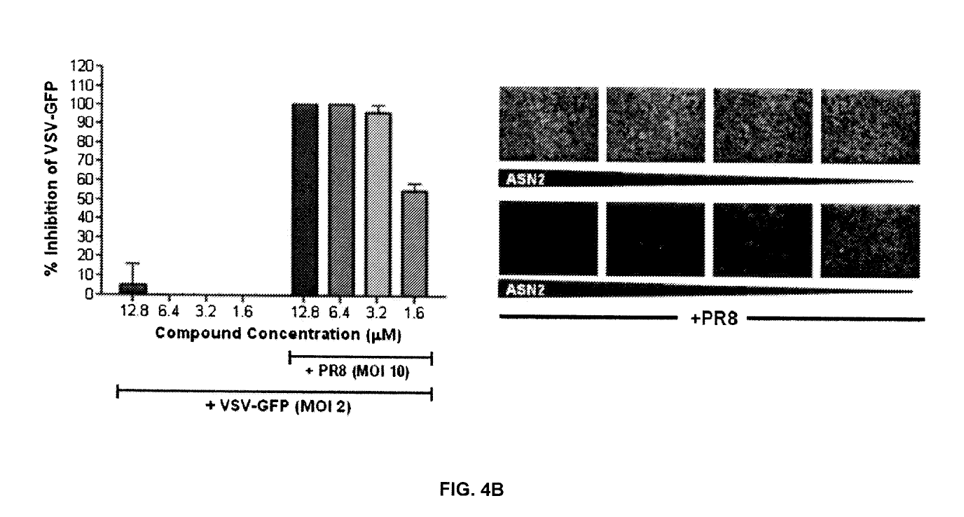

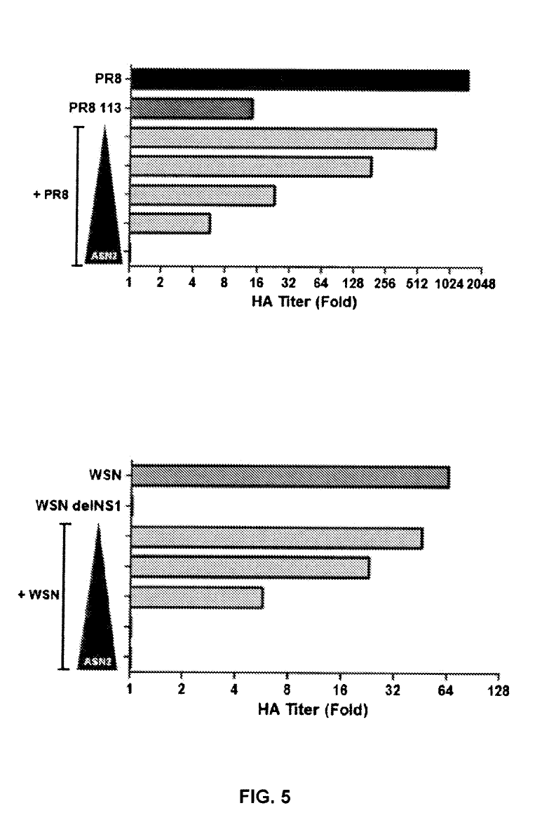

In order to verify that the reporter induction observed in the screening assay is consistent with actual IFN protein production, an IFN bioassay was performed. In this assay, Compound treated MDCK cells were subsequently mock infected or infected with A/PR/8/34 virus. Supernatants were collected and after UV inactivation, they were added to fresh MDCK cells in order to induce an antiviral state if IFN was present. After 24 hours incubation, the cells were infected with VSV-GFP, which is a virus sensitive to the effects of IFN. If the cells developed an antiviral state, VSV-GFP will not grow and no GFP signal will be detected. ASN2, a Compound that requires virus infection in order to induce IFN in the reporter assay, is able to induce biologically relevant levels of IFN once the Compound-treated cells are also infected with A/PR/8/34. This IFN produced is capable of inhibiting growth of VSV-GFP (FIG. 4).

6.1.2.3 Effect of Compounds on Influenza Virus Replication

Compounds that induce IFN, as defined by the interferon bioassay, were subsequently tested for their ability to inhibit multi-cycle replication of the A/PR/8/34 virus and A/WSN/33 influenza viruses. As a positive control for each virus, an NS1 mutant virus was used, A/PR/8/34 delNS1 and A/WSN/33 delNS1, respectively. ASN2 (identified in Table 1 as Compound 1), a Compound that requires virus infection in order to induce IFN, is able to attenuate both A/PR/8/34 and A/WSN/33 virus replication in a dose-dependent manner.

6.2 Characterization of the Anti-Viral Activity of ASN2

6.2.1 ASN2 Inhibits Multiple Influenza A Virus Subtypes

The ability of ASN2 (Compound 1) to inhibit the replication of multiple influenza

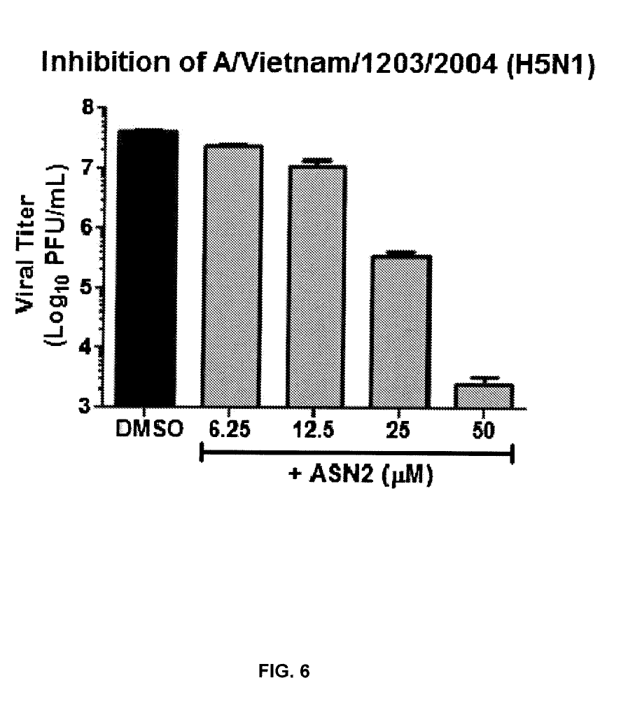

A virus subtypes was assessed using viral titer assays as described in Section 5.2.3.1.1, supra. Monolayers of A549 cells were infected with influenza virus at a multiplicity of 0.01 pfu and cultured in the presence or absence of various dilutions of ASN2 for 2 days. FIG. 6 demonstrates the antiviral activity of ASN2 against influenza A strain A/Vietnam/1203/2004 (H5N1). FIG. 7 demonstrates the antiviral activity of ASN2 against multiple influenza A virus strains. The 50 percent inhibitory concentration of the compound (IC₅₀) as well as the 50 percent cytotoxic activity (CC₅₀) of the compound were determined and the selective index for specific viruses is presented. As demonstrated in FIG. 7, ASN2 has broad antiviral activity against influenza A virus subtypes. In addition, ASN2 capably inhibits replication of influenza A virus subtypes across many generations, i.e., ASN2 inhibits replication of influenza A virus subtypes from the year 1918 to the year 2009, as well as influenza A virus subtypes from intervening years (FIG. 7).

6.2.2 ASN2 Affects the Influenza Virus Replication Machinery

Using an influenza virus mini-genome assay, it was determined that ASN2 affects the replication machinery of influenza virus.

T or A549 cells were seeded into 12-well plates at 2×10⁵ cells per well and incubated overnight at 37° C. and 5% CO₂. Next, the cells were transfected using Lipofectamine-2000 (Invitrogen) with pCAGGS protein expression vectors encoding the PB1, PB2 and PA subunits of the viral polymerase (100 ng of each) and the nucleocapsid protein (200 ng) of influenza virus strains A/WSN/33. The transfection mix also contained the RNA polymerase II driven Renilla luciferase reporter pRLTK (Promega) (200 ng) to normalize for transfection efficiency as well as the influenza virus-specific, RNA polymerase I driven, firefly luciferase reporter (pPolI Luc) (150 ng). Cells were cultured in DMEM which was supplemented 4 hours prior to the transfection with ASN2 or DMSO. The transfection was performed in OptiMEM (Invitrogen), which was also supplemented with ASN2 or DMSO. The OptiMEM was replaced 4 hours post transfection with DMEM containing ASN2 or DMSO. After a 20-24 hour incubation period at 37° C. and 5% CO₂, cells were harvested and lysed using the passive lysis buffer of the Dual Luciferase Assay Kit (Promega). Luminescence of firefly luciferase and Renilla luciferase was subsequently measured using the Dual Luciferase Assay Kit according to the specifications of the manufacturer.

As demonstrated in FIG. 8, luciferase activity in A549 cells decreased with increasing concentrations of ASN2, indicating that the compound affects the replication machinery of influenza virus.

6.2.3 The Antiviral Activity of ASN2 is Type I Interferon Independent

Viral titer assays (see, e.g., Section 5.2.3.1.1, supra) using cells that are type I interferon competent and type I interferon deficient demonstrated that the antiviral activity of ASN2 does not require the presence of type I interferon.

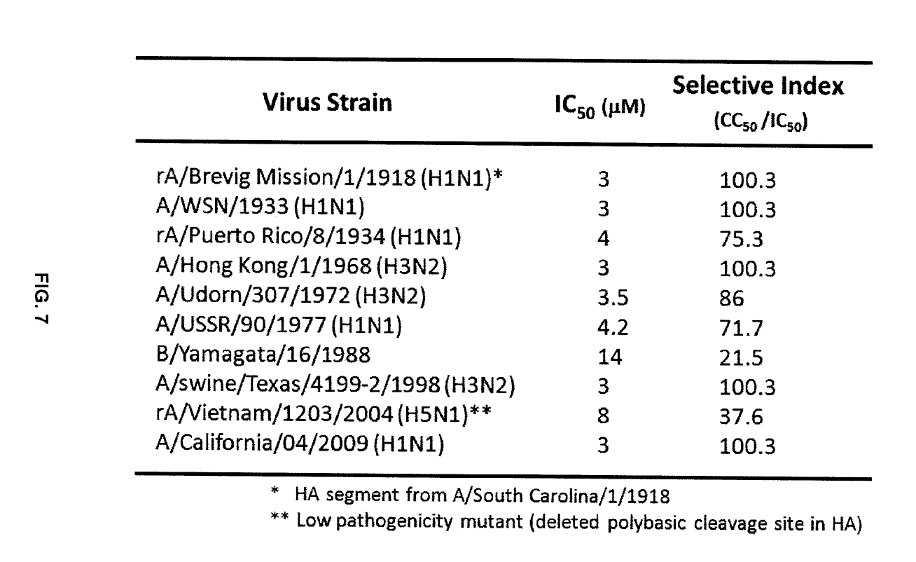

Monolayers of MDCK (type I interferon competent), A549 (type I interferon competent), and Vero (type I interferon deficient) cells were infected with influenza virus at a multiplicity of 0.01 pfu and cultured in the medium comprising 50 μM ASN2 or DMSO for 2 days. As shown in FIG. 9, ASN2 was capable of inhibiting viral replication in each cell type, thus indicating that the presence of interferon is not required for the antiviral activity of ASN2 and that ASN2 is effective at inhibiting viral replication in both human and non-human cells.

6.3 Characterization Virus-Independent Interferon-Inducing Compounds

As demonstrated above (see Section 6.1.2.2), multiple compounds were identified that induce interferon production by MDCK cells independent of virus infection. The interferon-inducing ability of these compounds, as well as their antiviral activity in human and non-human cells was further assessed.

6.3.1 Interferon Induction and Cytotoxicity

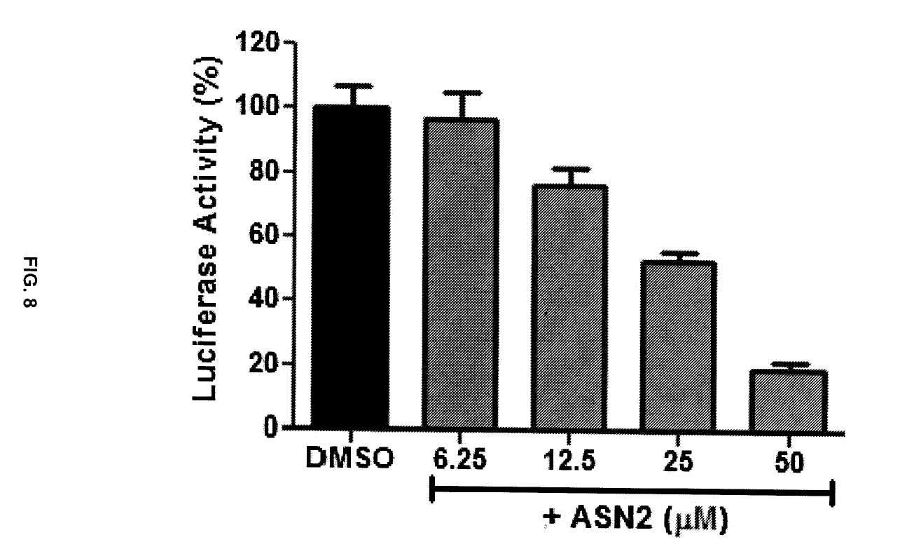

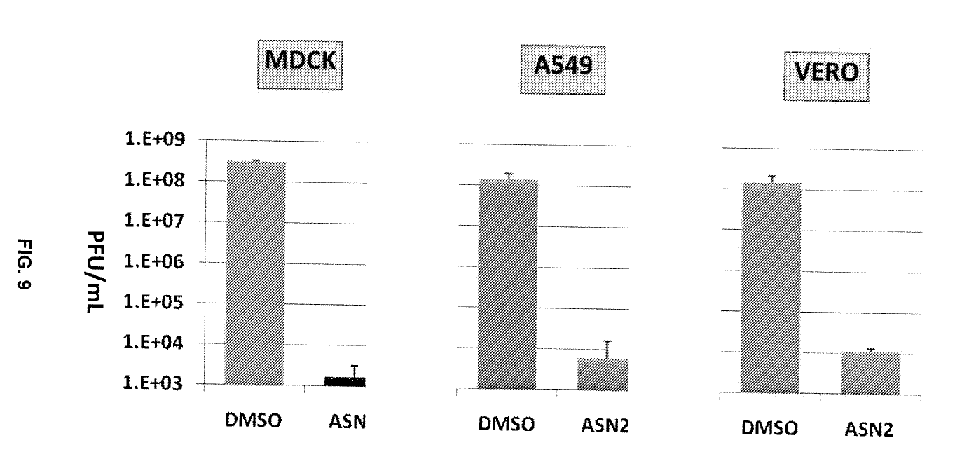

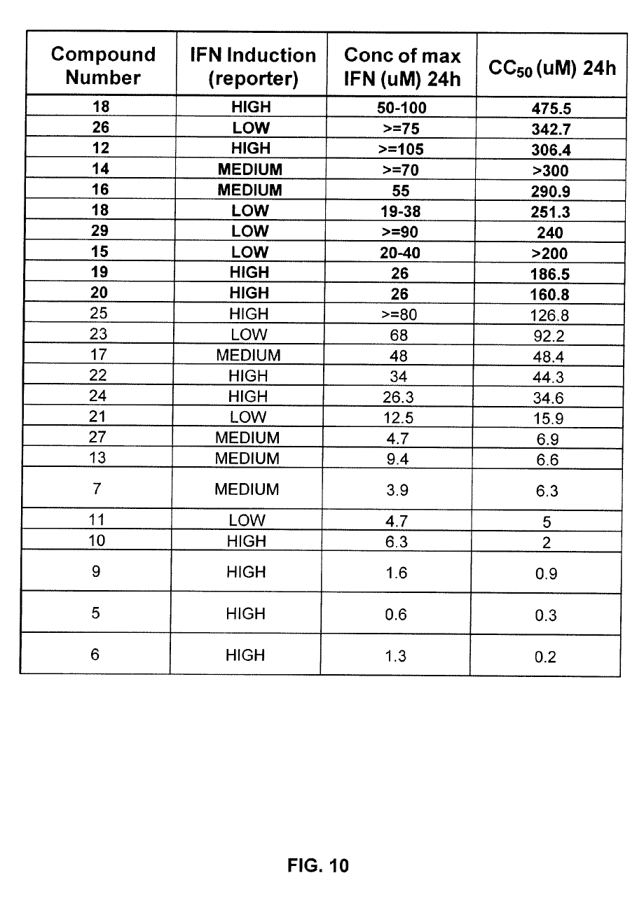

Interferon induction for the 24 identified virus-independent interferon-inducing compounds was assessed using an MDCK cell luciferase reporter system, as described in Sections 5.2.1 and 6.1.1.3, supra; cytotoxicity of the compounds was determined using the CellTiter-Glo®Luminescent Cell Viability Assay (Promega Corp., Madison, Wis.), as described in Sections 5.2.2 and 6.1.1.5, supra. As demonstrated in FIG. 10, several compounds possessed the ability to induce interferon while possessing minimal cytotoxicity.

6.3.2 Antiviral Activity in MDCK Cells

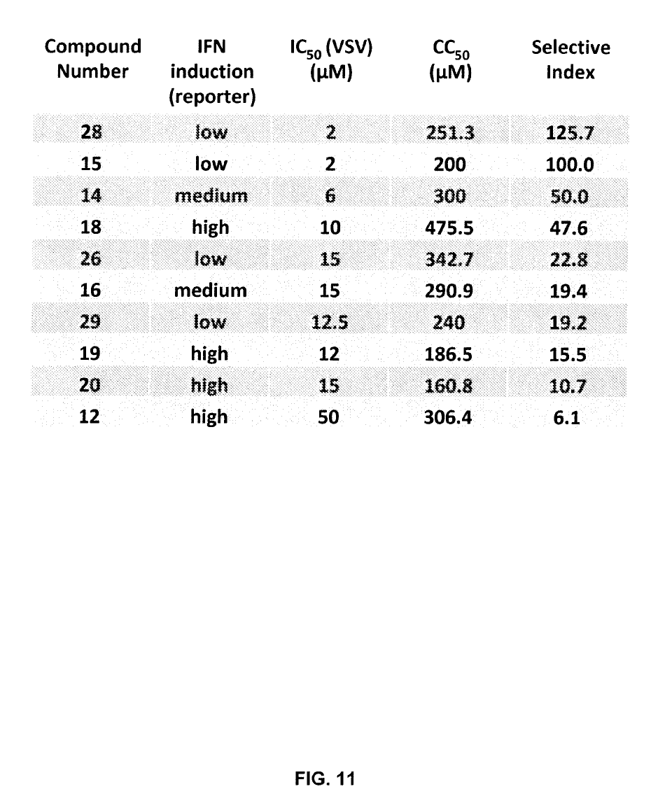

Several of the compounds identified in Section 6.3.1 as being able to induce interferon and having low cytotoxicity were analyzed for their antiviral activity against vesicular stomatitis virus (VSV) as described in Section 6.1.1.7, supra. Again, cytotoxicity of the compounds was determined using the CellTiter-Glo® Luminescent Cell Viability Assay (Promega Corp., Madison, Wis.), as described in Sections 5.2.2 and 6.1.1.5, supra. As demonstrated in FIG. 11, each these compounds possessed antiviral activity.

6.3.3 Interferon Induction and Antiviral Activity in Human Cells

The ability of the 24 compounds determined to be capable of inducing interferon in MDCK cells in virus-independent fashion to induce interferon in human cells was assessed.

Interferon induction by the 24 compounds in 293T and A549 cells was assessed using a luciferase reporter system, as described in Sections 5.2.1 and 6.1.1.3, supra. Antiviral activity of the 24 compounds in 293T and A549 cells was assessed by infecting the cells with vesicular stomatitis virus (VSV) and analyzing the antiviral effect of the compounds as described in Section 6.1.1.7, supra.

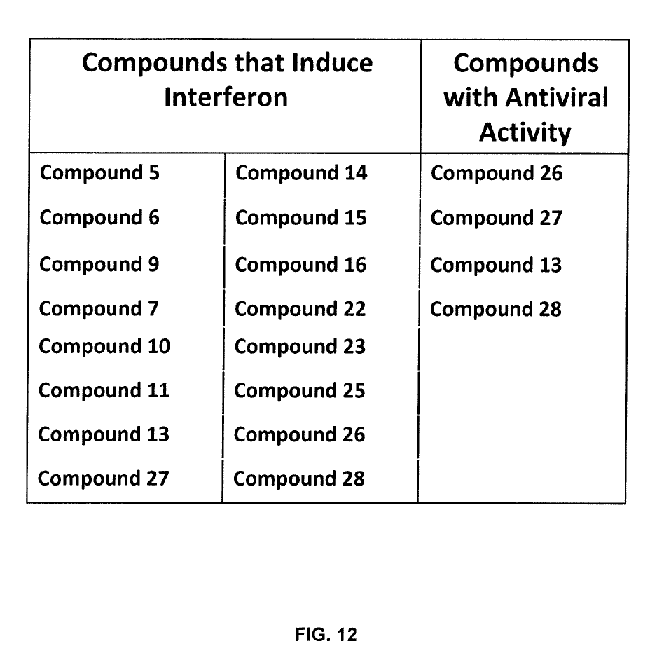

As shown in FIG. 12, sixteen of the compounds analyzed possessed the ability to induce interferon in each of the human cell types. Of these sixteen compounds, four possess antiviral activity in human cells (see FIG. 12).

### 7. EQUIVALENTS

The foregoing is not to be limited in scope by the specific embodiments described herein. Indeed, various modifications of the antibodies and methods provided herein and their equivalents, in addition to those described herein will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1. A method for treating an influenza virus disease, comprising administering to a subject in need thereof an effective amount of a Compound, wherein the Compound has the structure: 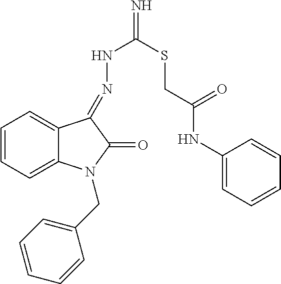
or a pharmaceutically acceptable salt or stereoisomer, including enantiomer, diastereomer, racemate or mixtures of stereoisomer, thereof.

2. The method of claim 1, wherein the influenza virus is an influenza A virus.

3. (canceled)

4. The method of claim 1, wherein the subject is human.

5. A method for treating an interferon-sensitive disease, comprising administering to a subject in need thereof an effective amount of a Compound, wherein the Compound has the structure: 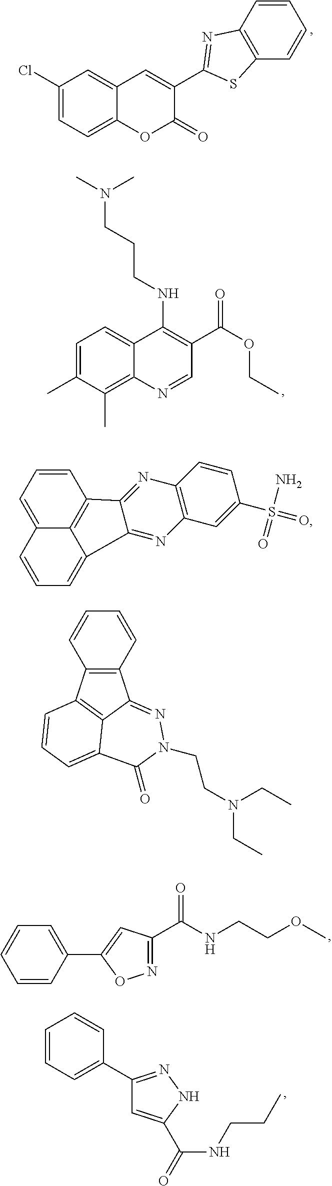 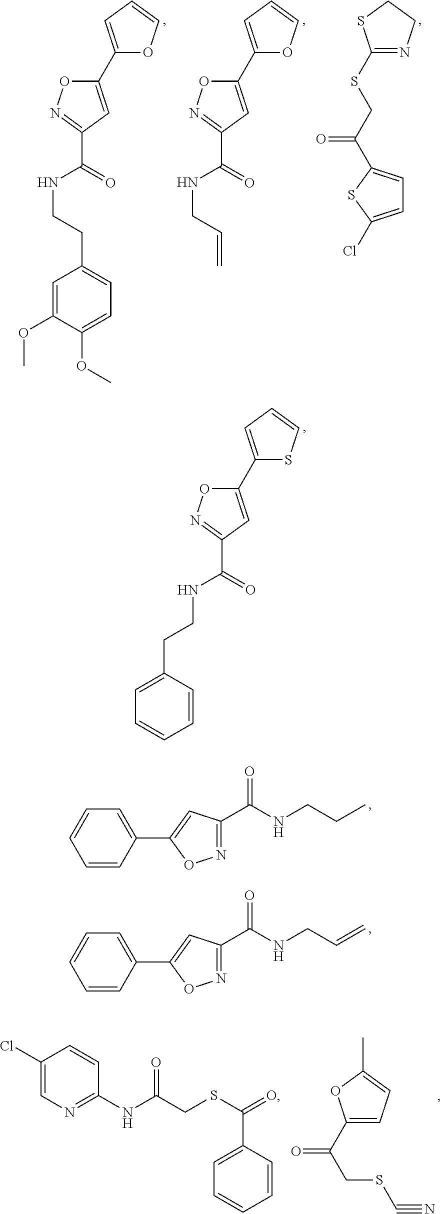 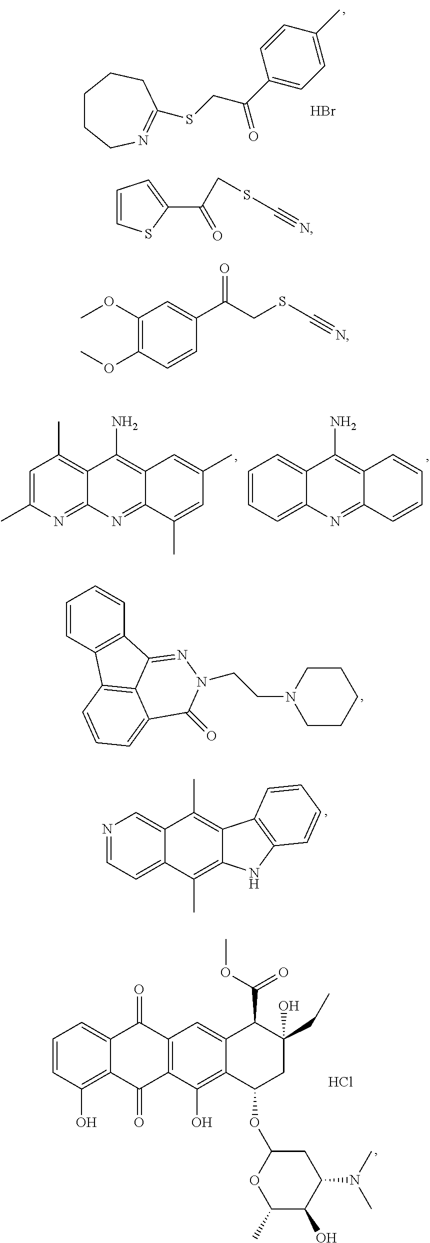 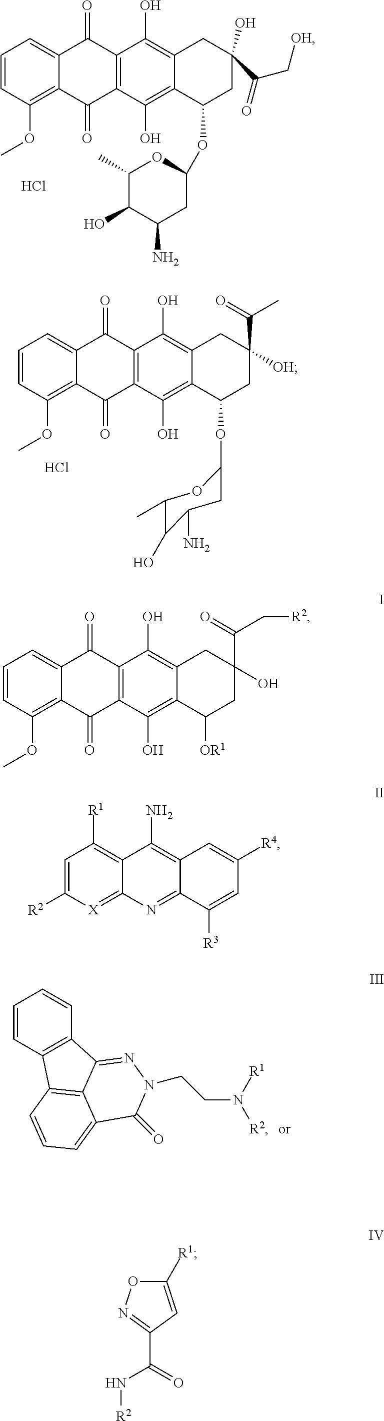
or a pharmaceutically acceptable salt or stereoisomer, including enantiomer, diastereomer, racemate or mixtures of stereoisomer, thereof, wherein
(i) R¹ of formula I is substituted or unsubstituted heterocyclyl; and R² of formula I is H or OH;
(ii) X of formula II is CH or N; and R¹, R², R³ and R⁴ of formula II are at each occurrence independently hydrogen or substituted or unsubstituted C₁₋₄alkyl;
(iii) R¹ and R² of formula III are at each occurrence independently substituted or unsubstituted C₁₋₄alkyl, or R¹ and R² of formula III taken together with the nitrogen atom to which they are attached form substituted or unsubstituted heterocyclyl; and
(iv) R¹ of formula IV is substituted or unsubstituted aryl or substituted or unsubstituted heteroaryl; and R² of formula IV is substituted or unsubstituted C₁₋₄alkyl, substituted or unsubstituted C₂₋₄alkenyl or substituted or unsubstituted heteroaryl,
wherein the compound of formula I is not doxorubicin or daunorubicin and the compound of formula II is not aminacrine.

6. (canceled)

7. (canceled)

8. The method of claim 5, wherein
(i) R¹ of formula I is substituted tetrahydro-2H-pyran;
(ii) R¹, R², R³ and R⁴ of formula II are methyl;
(iii) R¹ and R² of formula III are each ethyl, or R¹ and R² of formula III taken together with the nitrogen atom to which they are attached form piperidine; and
(iv) R¹ of formula IV is substituted or unsubstituted furan, thiophene or phenyl; and R² of formula IV is substituted or unsubstituted alkoxyalkyl, arylalkyl, allyl, propyl or alkoxyalkyl.

9. The method of claim 5, wherein the subject is human.

10. The method of claim 5, wherein the interferon-sensitive disease is caused by a viral infection, wherein the interferon-sensitive disease is caused by a bacterial infection, wherein the interferon-sensitive disease is multiple sclerosis, or wherein the interferon-sensitive disease is cancer.

11. The method of claim 10, wherein the virus is influenza virus, adenovirus, arbovirus, paramyxovirus, baculovirus, coronavirus, papillomavirus, parvovirus, chickenpox virus, reovirus, Ebola virus, Ebola-like virus, echo virus, encephalitis virus, filovirus, hantavirus, hepatitis virus, German measles virus, cytomegalovirus, hemorrhagic fever virus, herpes simplex virus, hepatitis B virus, hepatitis C virus, human immunodeficiency virus, human papillomavirus, human T cell leukemia virus, human T cell lymphoma virus, human T cell lymphotropic virus, Lassa fever virus, Marburg virus, measles virus, mumps virus, myxovirus, nairovirus, nanirnavirus, nariva virus, ndumo virus, Necrovirus, neethling virus, neopvirus, neurotropic virus, Newcastle disease virus, oncornavirus, orbivirus, orthomyxovirus, parainfluenza virus, paramyxovirus, parvovirus, picornavirus, rabies virus, respiratory syncytial virus, rhinovirus, rubella virus, rubeola virus, SARS virus, Sendai virus, simian immunodeficiency virus, simian parainfluenza virus, smallpox virus, varicella zoster virus, variola virus, or vesicular stomatitis virus.

12. (canceled)

13. (canceled)

14. (canceled)

15. A method of enhancing the immune response in a subject that has been administered a vaccine, comprising administering to the subject an effective amount of a Compound, wherein the Compound has the formula: 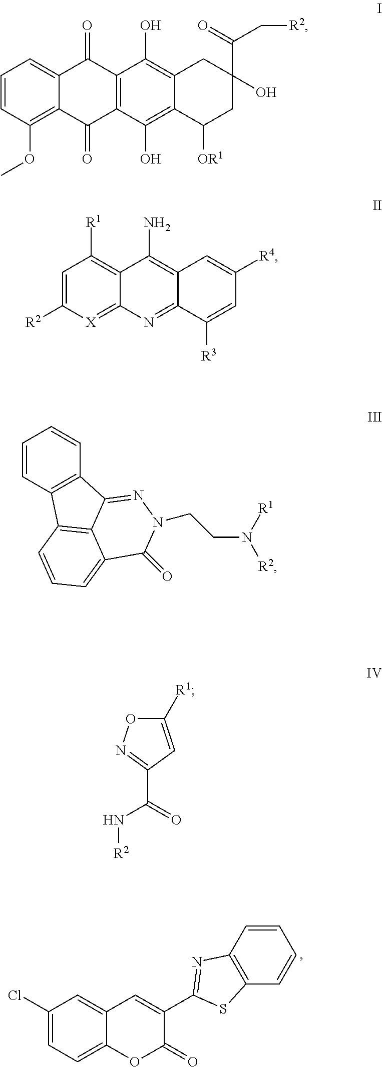 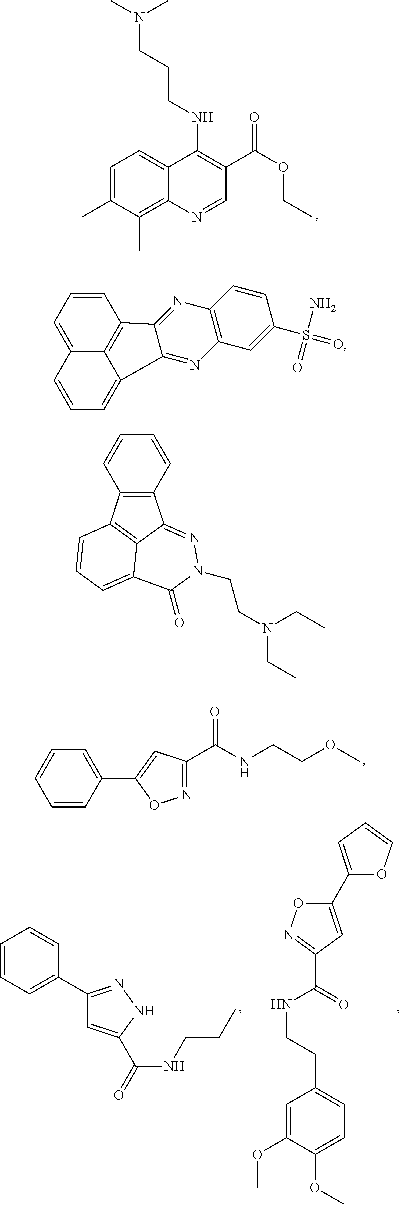 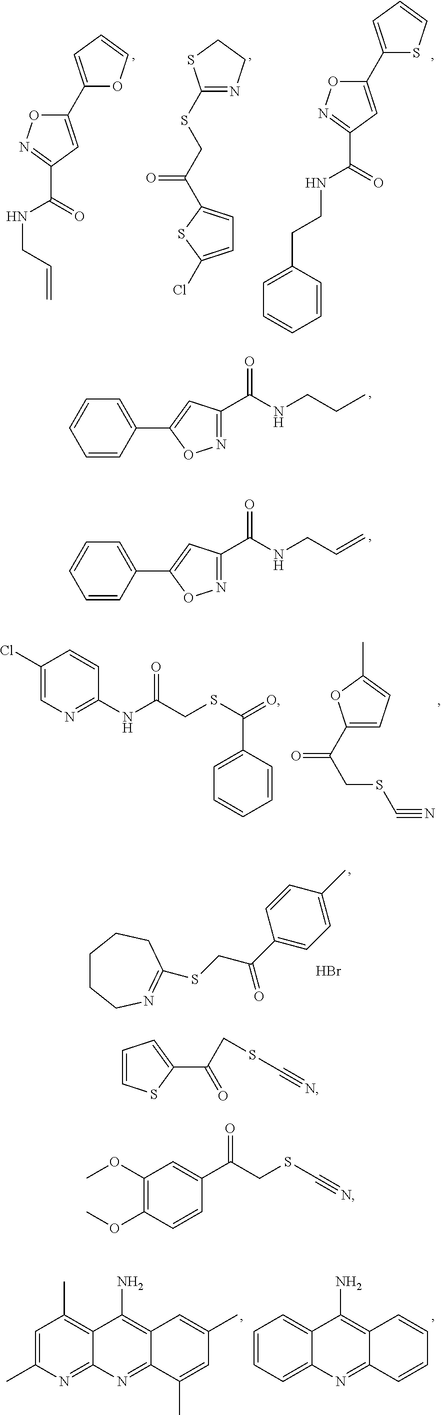 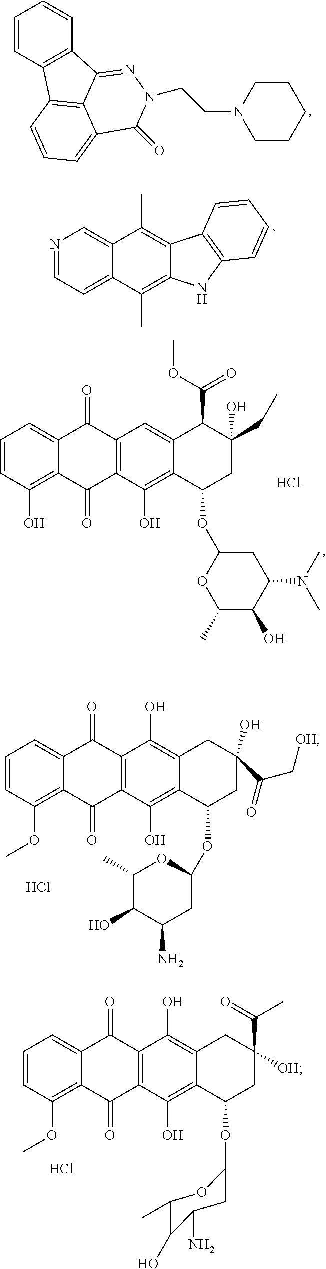 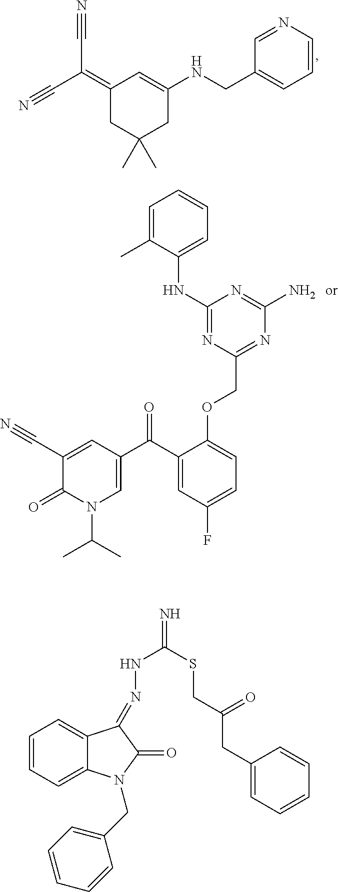
or a pharmaceutically acceptable salt or stereoisomer, including enantiomer, diastereomer, racemate or mixtures of stereoisomer, thereof; wherein
(i) R¹ of formula I is substituted or unsubstituted heterocyclyl; and R² of formula I is H or OH;
(ii) X of formula II is CH or N; and R¹, R², R³ and R⁴ of formula II are at each occurrence independently hydrogen or substituted or unsubstituted C₁₋₄alkyl;
(iii) R¹ and R² of formula III are at each occurrence independently substituted or unsubstituted C₁₋₄alkyl, or R¹ and R² of formula III taken together with the nitrogen atom to which they are attached form substituted or unsubstituted heterocyclyl; and
(iv) R¹ of formula IV is substituted or unsubstituted aryl or substituted or unsubstituted heteroaryl; and R² of formula IV is substituted or unsubstituted C₁₋₄alkyl, substituted or unsubstituted C₂₋₄alkenyl or substituted or unsubstituted heteroaryl.

16. The method of claim 15, wherein
(i) R¹ of formula I is substituted tetrahydro-2H-pyran;
(ii) R¹, R², R³ and R⁴ of formula II are methyl;
(iii) R¹ and R² of formula III are each ethyl, or R¹ and R² of formula III taken together with the nitrogen atom to which they are attached form piperidine; and
(iv) R¹ of formula IV is substituted or unsubstituted furan, thiophene or phenyl; and R² of formula IV is substituted or unsubstituted alkoxyalkyl, arylalkyl, allyl, propyl or alkoxyalkyl.

17. The method of claim 15, wherein the subject is human.

18. The method of claim 15, wherein the vaccine is an anthrax vaccine, BCG vaccine, Diphtheria vaccine, tetanus vaccine, pertussis vaccine, haemophilus B vaccine, hepatitis A vaccine, hepatitis B vaccine, human papillomavirus vaccine, influenza vaccine, Japanese encephalitis virus vaccine, measles vaccine, mumps vaccine, plague vaccine, pneumococcal vaccine, poliovirus vaccine, rabies vaccine, respiratory syncytial virus vaccine, rotavirus vaccine, rubella vaccine, smallpox vaccine, typhoid vaccine, varicella virus vaccine, yellow fever vaccine, or zoster vaccine.

19. The method of claim 15, wherein the vaccine is a live virus vaccine.

20. The method of claim 19, wherein the subject is human.

21. The method of claim 19, wherein the vaccine is an anthrax vaccine, BCG vaccine, Diphtheria vaccine, tetanus vaccine, pertussis vaccine, haemophilus B vaccine, hepatitis A vaccine, hepatitis B vaccine, human papillomavirus vaccine, influenza vaccine, Japanese encephalitis virus vaccine, measles vaccine, mumps vaccine, plague vaccine, pneumococcal vaccine, poliovirus vaccine, rabies vaccine, respiratory syncytial virus vaccine, rotavirus vaccine, rubella vaccine, smallpox vaccine, typhoid vaccine, varicella virus vaccine, yellow fever vaccine, or zoster vaccine.

22. The method of claim 15, wherein the vaccine is a live influenza virus vaccine.

23. The method of claim 22, wherein the subject is human.

24. (canceled)

25. (canceled)

26. (canceled)

27. (canceled)

28. (canceled)

29. (canceled)

30. A method for treating or preventing a viral disease in a subject in need thereof, comprising administering to the subject an effective amount of a Compound, wherein the Compound has the structure: 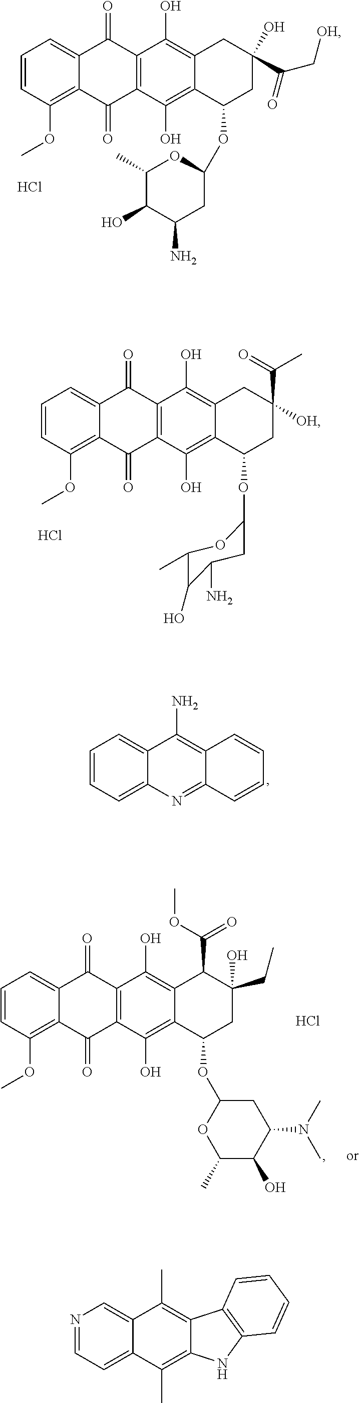
or a pharmaceutically acceptable salt or stereoisomer, including enantiomer, diastereomer, racemate or mixtures of stereoisomer, thereof.

31. The method of claim 30, wherein the subject is human.

32. The method of claim 30, wherein the viral disease is caused by influenza virus, adenovirus, arbovirus, paramyxovirus, baculovirus, coronavirus, papillomavirus, parvovirus, chickenpox virus, reovirus, Ebola virus, Ebola-like virus, echo virus, encephalitis virus, filovirus, hantavirus, hepatitis virus, German measles virus, cytomegalovirus, hemorrhagic fever virus, herpes simplex virus, human immunodeficiency virus, human papillomavirus, human T cell leukemia virus, human T cell lymphoma virus, human T cell lymphotropic virus, Lassa fever virus, Marburg virus, measles virus, mumps virus, myxovirus, nairovirus, nanirnavirus, nariva virus, ndumo virus, Necrovirus, neethling virus, neopvirus, neurotropic virus, Newcastle disease virus, oncornavirus, orbivirus, orthomyxovirus, parainfluenza virus, paramyxovirus, parvovirus, picornavirus, rabies virus, respiratory syncytial virus, rhinovirus, rubella virus, rubeola virus, SARS virus, Sendai virus, simian immunodeficiency virus, simian parainfluenza virus, smallpox virus, varicella zoster virus, variola virus, or vesicular stomatitis virus.

33. The method of claim 30, wherein the subject is cancer-free.
